(19) United States
(12) Patent Application Publication
(10) Pub. No.: US 2024/0209330 A1
(43) Pub. Date: Jun. 27, 2024

(54) PHI29 POLYMERASE FUSION PROTEINS AND USES THEREOF

(71) Applicant: 10x Genomics, Inc., Pleasanton, CA (US)

(72) Inventors: Shankar SHASTRY, Pleasanton, CA (US); Robert Henley, Castro Valley, CA (US)

(21) Appl. No.: 18/545,941

(22) Filed: Dec. 19, 2023

Related U.S. Application Data
(60) Provisional application No. 63/434,027, filed on Dec. 20, 2022.

(51) Int. Cl. C12N 9/12; C07K 14/47; C12N 15/63; C12Q 1/6844

(52) U.S. Cl. CPC C12N 9/1252; C07K 14/47; C12N 15/63; C12Q 1/6844; C07K 2319/21; C12Y 207/07007

(57) ABSTRACT

The present disclosure relates in some aspects to compositions and methods of using Phi29 polymerase fusion proteins. In some aspects, the compositions and methods provided herein address certain limitations associated with the Phi29 polymerase. In some aspects, the composition and methods disclosed herein provide more robust nucleic acid amplification for improved in vitro and in situ analysis.

### CROSS-REFERENCE TO RELATED APPLICATIONS

This application claims priority to U.S. Provisional Patent Application No. 63/434,027, filed Dec. 20, 2022, entitled “PHI29 POLYMERASE FUSION PROTEINS AND USES THEREOF,” which is herein incorporated by reference in its entirety for all purposes.

### REFERENCE TO AN ELECTRONIC SEQUENCE LISTING

The content of the electronic sequence listing (202412017600SEQLIST.xml; Size: 71,432 bytes; and Date of Creation: Dec. 1, 2023) is herein incorporated by reference in its entirety.

### FIELD

The present disclosure relates in some aspects to compositions and methods of using fusion proteins comprising Phi29 polymerases.

### BACKGROUND

Phi29 DNA polymerase is a DNA polymerase used for methods of amplifying DNA such as rolling circle amplification (RCA). Existing Phi29 DNA polymerases can have certain limitations, which restrict their utility. Improved Phi29 polymerases are needed. Provided herein are compositions and methods that address such and other needs.

### SUMMARY

Provided herein is a fusion protein comprising a Phi29 polymerase domain and one or more protein-protein interaction domains.

In some of any embodiments, the one or more protein-protein interaction domains comprise one or more dimerization domains. In some embodiments, the fusion protein comprises a Phi29 polymerase domain and a dimerization domain of a different protein.

In some of any embodiments, the dimerization domain comprises a kinesin 1 dimerization domain.

Also provided herein is a fusion protein comprising a Phi29 polymerase domain and a kinesin 1 dimerization domain.

In some of any embodiments, the kinesin 1 dimerization comprises the sequence set forth in SEQ ID NO: 18 or 20 or a sequence that has at least at or about 90%, 95%, 96%, 97%, 98%, 99%, or 99.5% sequence identity to the sequence set forth in SEQ ID NO:18 or 20. In some of any embodiments, the kinesin 1 dimerization domain comprises the sequence set forth in SEQ ID NO:18.

In some of any embodiments, the one or more dimerization domains are located N-terminal and/or C-terminal of the Phi29 polymerase domain. In some of any embodiments, when a first fusion protein molecule and a second fusion protein molecule are present in proximity, the dimerization domains interact to produce a dimer comprising the first fusion protein molecule and the second fusion protein molecule.

In some of any embodiments, the one or more protein-protein interaction domains comprises a SpyCatcher domain.

Also provided herein is a fusion protein comprising a Phi29 polymerase domain and a SpyCatcher domain.

In some of any embodiments, the SpyCatcher domain comprises the sequence set forth in SEQ ID NO:22 or a sequence that has at least at or about 90%, 95%, 96%, 97%, 98%, 99%, or 99.5% sequence identity to the sequence set forth in SEQ ID NO:22. In some of any embodiments, the SpyCatcher domain comprises the sequence set forth in SEQ ID NO:22.

In some of any embodiments, the SpyCatcher domain is located N-terminal and/or C-terminal of the Phi29 polymerase domain.

In some of any embodiments, the one or more protein-protein interaction domains comprises a Spy Tag domain.

Also provided herein is a fusion protein comprising a Phi29 polymerase domain and a SpyTag domain.

In some of any embodiments, the SpyTag domain comprises the sequence set forth in SEQ ID NO:24 or a sequence that has at least at or about 90%, 95%, 96%, 97%, 98%, 99%, or 99.5% sequence identity to the sequence set forth in SEQ ID NO:24. In some of any embodiments, the SpyTag domain comprises the sequence set forth in SEQ ID NO:24.

In some of any embodiments, the Spy Tag domain is located N-terminal and/or C-terminal of the Phi29 polymerase domain.

In some of any embodiments, when a first fusion protein molecule comprising a SpyCatcher domain and a second fusion protein molecule comprising a SpyTag domain are present in proximity, the SpyCatcher domain and the SpyTag domain interact to form a covalent bond to produce a dimer comprising the first fusion protein molecule and the second fusion protein molecule.

In some of any embodiments, the Phi29 polymerase domain comprises the sequence set forth in SEQ ID NO:3 or a sequence that has at least at or about 95%, 96%, 97%, 98%, 99%, or 99.5% sequence identity to the sequence set forth in SEQ ID NO:3. In some of any embodiments, the Phi29 polymerase domain comprises the sequence set forth in SEQ ID NO:3. In some of any embodiments, the Phi29 polymerase domain comprises the sequence set forth in SEQ ID NO:1 or a sequence that has at least at or about 95%, 96%, 97%, 98%, 99%, or 99.5% sequence identity to the sequence set forth in SEQ ID NO:1. In some of any embodiments, the Phi29 polymerase domain comprises the sequence set forth in SEQ ID NO:1.

In some of any embodiments, the Phi29 polymerase domain comprises one or more amino acid substitutions at one or more positions selected from among positions 68 and 83, corresponding to the positions of the sequence set forth in SEQ ID NO:1. In some of any embodiments, the Phi29 polymerase domain further comprises one or more amino acid substitutions other than at positions 68 and 83, corresponding to the positions of the sequence set forth in SEQ ID NO:1. In some of any embodiments, the Phi29 polymerase domain comprises one or more amino acid substitutions at one or more positions selected from among positions 131, 135, 142, 148, 224, 239, 250, 253, 306, 308, 375, 437, 466, 476, 484, 508, 510, 512, 515, 539, 570, and 571, corresponding to the positions of the sequence set forth in SEQ ID NO:1. In some of any embodiments, the Phi29 polymerase domain further comprises one or more amino acid substitutions other than at positions 131, 135, 142, 148, 224, 239, 250, 253, 306, 308, 375, 437, 466, 476, 484, 508, 510, 512, 515, 539, 570, and 571, corresponding to the positions of the sequence set forth in SEQ ID NO:1.

In some of any embodiments, the amino acid substitution at position 68 is selected from among A68S, A68C, A68N, A68P, A68T, A68W, and A68Y. In some of any embodiments, the amino acid substitution at position 68 is A68S. In some of any embodiments, the amino acid substitution at position 83 is selected from among A83E, A83D, A83G, A83K, A83Q, and A83V. In some of any embodiments, the amino acid substitution at position 83 is A83E.

In some of any embodiments, the Phi29 polymerase domain comprises the sequence set forth in SEQ ID NO:7 or a sequence that has at least at or about 95%, 96%, 97%, 98%, 99%, or 99.5% sequence identity to the sequence set forth in SEQ ID NO:7. In some of any embodiments, the Phi29 polymerase domain comprises the sequence set forth in SEQ ID NO:7.

In some of any embodiments, the Phi29 polymerase domain further comprises an N-terminal deletion of the methionine residue at position 1, corresponding to the positions of the sequence set forth in SEQ ID NO:1.

In some of any embodiments, the fusion protein further comprises one or more heterologous nucleic acid binding domains, linkers, and/or tags.

In some of any embodiments, the fusion protein further comprises one or more heterologous nucleic acid binding domains.

Provided herein is a fusion protein comprising a Phi29 polymerase domain and one or more heterologous nucleic acid binding domains.

In some of any embodiments, the one or more heterologous nucleic acid binding domains is selected from among a HU domain, a helix-hairpin-helix (HhH) DNA binding motif, a helix-hairpin-helix fusion [(HhH)₂] DNA binding motif, a single stranded DNA binding domain, an SD07 DNA binding motif, an SS07 DNA binding motif. In some of any embodiments, the one or more heterologous nucleic acid binding domains comprises a HU domain.

Provided herein is a fusion protein comprising a Phi29 polymerase domain and one or more HU domains. In some of any embodiments, the HU domain comprises a bacterial HU domain. In some of any embodiments, the HU domain comprises an E. coli HU domain. In some embodiments, the HYU domain comprises a HU domain from a HuA/HuR, a HuB, a HuC or a HuD protein.

In some of any embodiments, the HU domain comprises the sequence set forth in SEQ ID NO:26 or a sequence that has at least at or about 90%, 95%, 96%, 97%, 98%, 99%, or 99.5% sequence identity to the sequence set forth in SEQ ID NO:26. In some of any embodiments, the HU domain comprises the sequence set forth in SEQ ID NO:26.

In some of any embodiments, the HU domain is located N-terminal and/or C-terminal of the Phi29 polymerase domain. In some of any embodiments, the fusion protein further comprises one or more tags. In some of any embodiments, the tag is selected from among a poly histidine (HIS) tag, a solubility enhancement tag (SET), a small ubiquitin modified (SUMO) tag, a Fasciola hepatica 8-kDa antigen (Fh8) tag, a thioredoxin (Trx) tag, a glutathione-s-transferase (GST) tag, a maltose-binding protein (MBP) tag, an IgG domain B1 of protein G (GB1) tag, a mutated dehalogenase (Halo) tag, a steptavidin-binding peptide (SBP) tag, and a Tamavidin tag.

In some of any embodiments, the tag is a HIS tag. In some of any embodiments, the tag is a HIS tag and comprises the sequence set forth in SEQ ID NO:43 or SEQ ID NO:44. In some of any embodiments, the tag is a SUMO tag. In some of any embodiments, the tag is a SUMO tag and comprises the sequence set forth in SEQ ID NO:41. In some of any embodiments, the tag is a SET tag. In some of any embodiments, the tag is a SET tag and comprises the sequence set forth in SEQ ID NO:46 or SEQ ID NO:47.

In some of any embodiments, the fusion protein further comprises one or more linkers. In some of any embodiments, the linker is at least at or about 1, 2, 3, 4, 5, 6, 7, 8, 9, 10, 15, 20, 25, 30, 40, or 50 amino acids in length. In some of any embodiments, the linker comprises a sequence selected from the sequence set forth in any of SEQ ID NOS:9-17 and 48. In some of any embodiments, the linker is a cleavable linker.

In some of any embodiments, the fusion protein comprises the sequence set forth in SEQ ID NO:28 or 33 or has at least at or about 90%, 95%, 96%, 97%, 98%, 99%, or 99.5% sequence identity to the sequence set forth in SEQ ID NO:28 or 33. In some of any embodiments, the fusion protein comprises the sequence set forth in SEQ ID NO:28. In some of any embodiments, the fusion protein comprises the sequence set forth in SEQ ID NO:33.

In some of any embodiments, the fusion protein comprises the sequence set forth in SEQ ID NO:49 or has at least at or about 90%, 95%, 96%, 97%, 98%, 99%, or 99.5% sequence identity to the sequence set forth in SEQ ID NO:49. In some of any embodiments, the fusion protein comprises the sequence set forth in SEQ ID NO:49.

In some of any embodiments, the fusion protein comprises the sequence set forth in SEQ ID NO:34 or has at least at or about 90%, 95%, 96%, 97%, 98%, 99%, or 99.5% sequence identity to the sequence set forth in SEQ ID NO:34. In some of any embodiments, the fusion protein comprises the sequence set forth in SEQ ID NO:34.

In some of any embodiments, the fusion protein comprises the sequence set forth in SEQ ID NO:36 or has at least at or about 90%, 95%, 96%, 97%, 98%, 99%, or 99.5% sequence identity to the sequence set forth in SEQ ID NO:36. In some of any embodiments, the fusion protein comprises the sequence set forth in SEQ ID NO:36.

In some of any embodiments, the fusion protein comprises the sequence set forth in SEQ ID NO:38 or 39 or has at least at or about 90%, 95%, 96%, 97%, 98%, 99%, or 99.5% sequence identity to the sequence set forth in SEQ ID NO:38 or 39. In some of any embodiments, the fusion protein comprises the sequence set forth in SEQ ID NO:38. In some of any embodiments, the fusion protein comprises the sequence set forth in SEQ ID NO:39.

In some of any embodiments, the fusion protein exhibits one or more features selected from among improved amplification product homogeneity, improved solubility, improved nucleic acid binding affinity, improved processivity, improved fidelity, and/or improved polymerization rate, compared to a reference Phi29 polymerase. In some of any embodiments, the fusion protein exhibits improved amplification product homogeneity compared to a reference Phi29 polymerase. In some of any embodiments, the fusion protein generates a higher density of detected rolling-circle amplification (RCA) products in an RCA compared to a reference Phi29 polymerase. In some of any embodiments, the fusion protein generates a higher signal intensity of RCA products in an RCA compared to a reference Phi29 polymerase. In some of any embodiments, the fusion protein generates a smaller or more compact RCA product compared to a referenced Phi29 polymerase. In some of any embodiments, the fusion protein generates a more homogenous RCA products compared to a referenced Phi29 polymerase.

In some of any embodiments, the fusion protein exhibits improved solubility compared to a reference Phi29 polymerase. In some of any embodiments, the fusion protein exhibits improved nucleic acid binding affinity compared to a reference Phi29 polymerase. In some of any embodiments, the fusion protein exhibits improved processivity compared to a reference Phi29 polymerase. In some of any embodiments, the fusion protein exhibits improved fidelity compared to a reference Phi29 polymerase. In some of any embodiments, the fusion protein exhibits improved polymerization rate compared to a reference Phi29 polymerase.

In some of any embodiments, the RCA is performed at a temperature of at or about 5° C., 10° C., 15° C., 20° C., 25° C., 30° C., 35° C., 37° C., 40° ° C., 42° ° C., 45° C., 50° ° C. or higher.

In some of any embodiments, the reference Phi29 polymerase is a wild-type Phi29 polymerase, or a Phi29 polymerase that does not comprise the one or more protein-protein interaction domains or the one or more heterologous nucleic acid binding domains.

Also provided herein is a dimer comprising: a first fusion protein molecule comprising any of the provided fusion proteins, and a second fusion protein molecule comprising any of the provided fusion proteins.

Also provided herein is a composition comprising any of the provided fusion proteins or dimers.

In some of any embodiments, at least 70%, 75%, 80%, 85%, 90%, 95%, 96%, 97%, 98%, 99% or 99.5% of the polypeptides or proteins of the composition is the fusion protein or the dimer.

Also provided herein is a polynucleotide encoding any of the fusion proteins provided herein.

In some of any embodiments, the polynucleotide comprises the sequence set forth in SEQ ID NO:31 or a sequence that has at least at or about 90%, 95%, 96%, 97%, 98%, 99%, or 99.5% sequence identity to the sequence set forth in SEQ ID NO:31. In some of any embodiments, the polynucleotide comprises the sequence set forth in SEQ ID NO:31.

Provided herein is a polynucleotide encoding a fusion protein, wherein the polynucleotide comprises the sequence set forth in SEQ ID NO:35 or a sequence that has at least at or about 90%, 95%, 96%, 97%, 98%, 99%, or 99.5% sequence identity to the sequence set forth in SEQ ID NO:35. In some of any embodiments, the polynucleotide comprises the sequence set forth in SEQ ID NO:35. In some of any embodiments, the polynucleotide comprises the sequence set forth in SEQ ID NO:37 or a sequence that has at least at or about 90%, 95%, 96%, 97%, 98%, 99%, or 99.5% sequence identity to the sequence set forth in SEQ ID NO:37. In some of any embodiments, the polynucleotide comprises the sequence set forth in SEQ ID NO:37.

Provided herein is a polynucleotide encoding a fusion protein, wherein the polynucleotide comprises the sequence set forth in SEQ ID NO:39 or a sequence that has at least at or about 90%, 95%, 96%, 97%, 98%, 99%, or 99.5% sequence identity to the sequence set forth in SEQ ID NO:39. In some of any embodiments, the polynucleotide comprises the sequence set forth in SEQ ID NO:39.

Provided herein is a polynucleotide encoding a fusion protein, wherein the polynucleotide comprises the sequence set forth in SEQ ID NO:33 or a sequence that has at least at or about 90%, 95%, 96%, 97%, 98%, 99%, or 99.5% sequence identity to the sequence set forth in SEQ ID NO:33. In some of any embodiments, the polynucleotide comprises the sequence set forth in SEQ ID NO:33.

In some of any embodiments, the polynucleotide is codon-optimized for a recombinant expression system to generate the fusion protein.

Provided herein is a recombinant nucleic acid molecule comprising any of the provided polynucleotides.

In some of any embodiments, the recombinant nucleic acid further comprises a transcription regulatory sequence operatively linked with the polynucleotide. In some of any embodiments, the transcription regulatory sequence comprises a promoter selected from among a bacterial, viral, and mammalian promoter.

Provided herein is a vector comprising any of the provided polynucleotides or any of the provided recombinant nucleic acid molecules. In some of any embodiments, the vector comprises a plasmid, a viral vector, a cosmid, or a transposon.

Provided herein is a recombinant expression system comprising any of the provided polynucleotides, any of the provided recombinant nucleic acid molecules, or any of the provided vectors.

In some of any embodiments, the polynucleotide, the recombinant nucleic acid molecule, or the vector is for use in transforming the recombinant expression system to generate a transformed recombinant expression system. In some of any embodiments, the transformed recombinant expression system generates the fusion protein. In some of any embodiments, the recombinant expression system is a cell system or a cell-free system. In some of any embodiments, the recombinant expression system comprises one or more cell systems selected from among a bacterial cell, a fungal cell, an insect cell, and a mammalian cell. In some of any embodiments, the polynucleotide sequence encoding the fusion protein is codon-optimized for the recombinant expression system.

Provided herein is a method for amplifying a nucleic acid, the method comprising contacting a biological sample containing a target nucleic acid to be amplified with any of the provided fusion proteins or any of the provided dimers. In some of any embodiments, the nucleic acid is amplified by a rolling circle amplification (RCA).

Provided herein is a method of performing a rolling circle amplification (RCA), the method comprising contacting a biological sample containing a target nucleic acid to be amplified with any of the fusion proteins provided herein or any of the dimers provided herein, thereby amplifying the nucleic acid by RCA.

In some of any embodiments, the method of performing an RCA further comprises contacting the biological sample with a circularizable probe. In some of any embodiments, the circularizable probe comprises one or more hybridization regions and one or more barcode regions. In some of any embodiments, the one or more barcode sequences correspond to a sequence of the target nucleic acid. In some of any embodiments, the one or more hybridization regions binds to and/or is complementary to the target nucleic acid or a portion of the target nucleic acid.

In some of any embodiments, the method further comprises ligating the ends of the circularizable probe. In some of any embodiments, the method further comprises ligating the ends of the circularizable probe, wherein the ligation is templated by the binding of the hybridization regions to the target nucleic acid or portion thereof, thereby generating a circularized probe. In some of any embodiments, the circularized probe is amplified by rolling circle amplification, to generate a rolling circle amplification product. In some of any embodiments, the rolling circle amplification product comprises multiple copies of a sequence complementary to the circularized probe.

In some of any embodiments, the method further comprises detecting the rolling circle amplification product. In some of any embodiments, the detecting comprises detecting a sequence in rolling circle amplification product, sequencing all or a portion of a sequence in rolling circle amplification product and/or in situ hybridization to a sequence in rolling circle amplification product. In some of any embodiments, the detecting comprises sequencing the complement of the one or more barcode sequences. In some of any embodiments, the detecting comprises hybridizing to the rolling circle amplification product a detection oligonucleotide labeled with a fluorophore, an isotope, a mass tag, or a combination thereof. In some of any embodiments, the detecting comprises imaging the rolling circle amplification product. In some of any embodiments, the amplification is performed in situ in the biological sample. In some of any embodiments, the detecting is performed in situ in the biological sample. In some of any embodiments, the biological sample is a fixed and/or permeabilized biological sample. In some of any embodiments, the biological sample is a tissue sample. In some of any embodiments, the target nucleic acid comprises deoxyribonucleotide residues. In some of any embodiments, the target nucleic acid comprises one or more ribonucleotide residues.

In some of any embodiments, the fusion protein and the biological sample are contacted at a temperature of at or about 5° C., 10° C., 15° C., 20° C., 25° C., 30° C., 35° ° C., 37° C., 40° C., 42° ° C., 45° C., 50° C. or higher. In some of any embodiments, the RCA is performed for at or about 15 minutes, 30 minutes, 45 minutes, 1 hour, 2 hours, 3 hours, 6 hours, 12 hours, or longer.

In some of any embodiments, the fusion protein or the dimer exhibits one or more features selected from among improved amplification product homogeneity, improved solubility, improved nucleic acid binding affinity, improved processivity, improved fidelity, and improved polymerization rate, compared to a reference Phi29 polymerase. In some of any embodiments, the fusion protein or the dimer exhibits improved amplification product homogeneity compared to a reference Phi29 polymerase.

In some of any embodiments, the fusion protein or the dimer generates a higher density of detected rolling-circle amplification (RCA) products in an RCA compared to a reference Phi29 polymerase. In some of any embodiments, the fusion protein or the dimer generates a higher signal intensity of RCA products in an RCA compared to a reference Phi29 polymerase. In some of any embodiments, the fusion protein or the dimer generates a smaller or more compact RCA product compared to a referenced Phi29 polymerase. In some of any embodiments, the fusion protein or the dimer generates a more homogenous RCA products compared to a referenced Phi29 polymerase.

In some of any embodiments, the fusion protein or the dimer exhibits improved solubility compared to a reference Phi29 polymerase. In some of any embodiments, the fusion protein or the dimer exhibits improved nucleic acid binding affinity compared to a reference Phi29 polymerase. In some of any embodiments, the fusion protein or the dimer exhibits improved processivity compared to a reference Phi29 polymerase. In some of any embodiments, the fusion protein or the dimer exhibits improved fidelity compared to a reference Phi29 polymerase. In some of any embodiments, the fusion protein or the dimer exhibits improved polymerization rate compared to a reference Phi29 polymerase.

In some of any embodiments, the RCA is performed at a temperature of at or about 5° C., 10° C., 15° C., 20° C., 25° C., 30° C., 35° C., 37° C., 40° C., 42° C., 45° C., 50° ° C. or higher.

In some of any embodiments, the reference Phi29 polymerase is a wild-type Phi29 polymerase, a Phi29 polymerase that does not comprise the one or more protein-protein interaction domains or the one or more heterologous nucleic acid binding domains, or a wild-type Phi29 polymerase monomer.

Also provided herein is a kit comprising: (1) any of the provided fusion proteins, any of the provided dimers, any of the provided compositions, any of the provided nucleotides, any of the provided recombinant nucleic acids, any of the provided vectors, and (2) one or more of: (a) dNTPs; (b) one or more di-cation; (c) reaction buffer; (d) a buffer for use with any of (a)-(c); and (e) instructions for use.

In some of any embodiments, the kit further comprises one or more primers. In some of any embodiments, the kit further comprises one or more circularizable probes. In some of any embodiments, the kit is for amplification of nucleic acid. In some of any embodiments, the kit further comprises a detection probe for detection of the amplified nucleic acid.

### BRIEF DESCRIPTION OF THE DRAWINGS

The drawings illustrate certain features and advantages of this disclosure. These embodiments are not intended to limit the scope of the appended claims in any manner.

FIGS. 1A-1C depict results from an in vitro rolling circle amplification (RCA) assay comparing activities of a wild-type (WT) Phi29 polymerase, a Phi29-dimer polymerase, and a Phi29-HU fusion polymerase. Shown in the FIGS. 1A-1C is a fluorescence readout for the amount of RCA product (RCP) generated as a function of polymerase concentration.

FIGS. 2A-2B depict results from an in vitro rolling circle amplification (RCA) assay comparing kinetic activities of a wild-type (WT) Phi29 polymerase, a Phi29-dimer polymerase, and a Phi29-HU fusion polymerase. Shown in FIG. 2A is a fluorescent readout for the amount of RCA product (RCP) generated across time at a polymerase concentration of 1 μM. FIG. 2B shows the average rate of RCP formation across time.

FIGS. 3A-3C depict results from in situ rolling circle amplification (RCA) assays comparing sensitivity of a wild-type (WT) Phi29 polymerase, a Phi29-dimer polymerase, and a Phi29-HU fusion polymerase. FIG. 3A shows RCA product (RCP) per nuclei in the first transcript.

FIG. 3B shows RCP per nuclei in the second transcript. FIG. 3C shows RCA product RCP per nuclei in the third transcript.

FIGS. 4A-4F depict results from in situ rolling circle amplification (RCA) assays comparing signal-to-noise ratio (SNR) and size of RCA product (RCP) of a wild-type (WT) Phi29 polymerase, a Phi29-dimer polymerase, and a Phi29-HU fusion polymerase. FIGS. 4A-4C show SNR for transcripts 1-3, respectively. FIGS. 4D-4F show size (μM) of RCP for transcripts 1-3, respectively.

FIGS. 5A-5F depict results from in situ rolling circle amplification (RCA) assays comparing intensity of RCA product (RCP) and signal-over-background (SOB) of a wild-type (WT) Phi29 polymerase, a Phi29-dimer polymerase, and a Phi29-HU fusion polymerase. FIGS. 5A-5C show signal intensity of RCP for transcripts 1-3, respectively. FIGS. 5D-5F show SOB of RCP for transcripts 1-3, respectively.

FIGS. 6A-6C depict results from in situ rolling circle amplification (RCA) assays comparing proportion of RCA product (RCP) of a wild-type (WT) Phi29 polymerase, a Phi29-dimer polymerase, and a Phi29-HU fusion polymerase. FIGS. 6A-6C show proportion of RCP for transcripts 1-3, respectively.

FIGS. 7A-7C depict results from in situ rolling circle amplification (RCA) assays comparing density of RCP of a wild-type (WT) Phi29 polymerase, a Phi29-dimer polymerase, and a Phi29-HU fusion polymerase. FIGS. 7A-7C show density of RCP for transcripts 1-3, respectively.

FIGS. 8A-8B depict results of an SDS-PAGE comparing the protein solubility of a Phi29 polymerase-SpyCatcher fusion protein having an amino terminal polyhistidine/SUMO tag (N-His/SUMO) (FIG. 8A) and a Phi29 polymerase-SpyTag fusion protein having an amino terminal polyhistidine/SUMO tag (N-His/SUMO) (FIG. 8B).

FIGS. 9A-9B depict results of an SDS-PAGE comparing the various fractions from nickel-column purification of a Phi29 polymerase-SpyCatcher fusion protein having an amino terminal polyhistidine/SUMO tag (N-His/SUMO) (FIG. 9A) and a Phi29 polymerase-SpyTag fusion protein having an amino terminal polyhistidine/SUMO tag (N-His/SUMO) (FIG. 9B).

FIGS. 10A-10C depict results of an SDS-PAGE comparing the molecular weight of a Phi29 polymerase-SpyCatcher fusion protein (FIG. 10A) and a Phi29 polymerase-SpyTag fusion protein (FIG. 10B); and a dimer of Phi29 polymerase-SpyCatcher:Phi29 polymerase-SpyTag (FIG. 10C) after protein coupling, before and after cleavage of the SUMO tag.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. OVERVIEW

Provided herein are fusion proteins comprising Phi29 polymerases. In some aspects, provided are fusion proteins comprising one or more polypeptide domains fused to a Phi29 polymerase (e.g., fusion Phi29 polymerases). In some aspects, the provided fusion polymerases comprise one or more protein-protein interaction domains, such as one or more dimerization domains, fused to a Phi29 polymerase. In some aspects, the provided fusion proteins comprise one or more heterologous nucleic acid binding domains, fused to a Phi29 polymerase. In some aspects, the provided fusion proteins comprising Phi29 polymerases exhibit improved properties, such as but not limited to, improved processivity, improved DNA binding affinity, improved stability, improved solubility, improved yield, improved ability to produce more uniform and/or more compact amplification product, improved sensitivity, and/or improved signal of detection of amplification product, compared to a wild-type Phi29 polymerase. Also provided are related polynucleotides, recombinant nucleic acid molecules, vectors, recombinant expression systems and kits, and methods for use and uses thereof.

Phi29 polymerase is a DNA polymerase used for amplifying DNA, including in methods such as rolling circle amplification (RCA). Wild-type or unmodified Phi29 polymerase has some disadvantages, such as limited processivity, solubility, DNA binding affinity, or a broad size distribution of amplification products. For example, typical RCA products generated with wild-type Phi29 is can exhibit a large variance in sizes, which can negatively affect detection and in situ visualization. In some contexts, wild-type Phi29 polymerase can result in low signal-to-noise ratio or signal-over-background, for amplification product detection. In some aspects, Phi29 polymerases with improved properties are needed, for example, for use in in situ hybridization and visualization of signal. Provided are fusion proteins comprising Phi29 polymerases that meet such needs.

In some aspects, the provided fusion Phi29 polymerases exhibit one or more improved features or properties compared to a reference Phi29 polymerase, such as a wild-type Phi29 polymerase or a Phi29 polymerase without the one or more polypeptide domains fused to it. In some aspects, the provided fusion Phi29 polymerases exhibit one or more features, such as improved processivity, improved DNA binding affinity, improved stability, improved solubility, improved yield, improved ability to produce more uniform and/or more compact amplification product, improved sensitivity, and/or improved signal of detection of amplification product, compared to a wild-type Phi29 polymerase, for example, compared to a reference Phi29 polymerase such as a wild-type Phi29 polymerase.

In some aspects, the provided embodiments are based on an observation that exemplary fusion Phi29 polymerases exhibited various improved properties for nucleic acid amplification, including improved solubility, improved processivity, improved nucleic acid affinity, increased RCA product (RCP) generation, and improved uniformity, for example as observed by a more uniform distribution of RCA products and higher signal intensity, compared to a reference Phi29 polymerase, in different types of assays and contexts for nucleic acid amplification, such as rolling circle amplification (RCA), both in in vitro assays and in situ assays, and using different types target genes.

In some aspects, the provided fusion Phi29 polymerases can be used to improve nucleic acid amplification, such as RCA, and result in improved analysis of biological samples, for example in vitro and in situ.

### II. Fusion Proteins

Provided herein are fusion proteins comprising Phi29 polymerases and one or more polypeptide domains, such as one or more protein-protein interaction domains or one or more heterologous nucleic acid binding domains. In some aspects, the fusion protein comprises a Phi29 polymerase and one or more protein-protein interaction domains, such as a dimerization domain. In some aspects, the fusion protein comprises a Phi29 polymerase and one or more heterologous nucleic acid binding domains. In some aspects, the fusion protein comprises a Phi29 polymerase, one or more protein-protein interaction domains, and one or more heterologous nucleic acid binding domains. In some aspects, the fusion protein also comprises one or more tags. In some embodiments, the fusion protein comprises a C-terminal protein-protein interaction domain, and one or more N-terminal tags. In some embodiments, the fusion protein comprises an N-terminal polyhistidine/SUMO tag. In some embodiments, the N-terminal polyhistidine/SUMO tag comprises, from the N-terminus, a polyhistidine sequence and a SUMO tag sequence. In some aspects, the fusion protein comprises a Phi29 polymerase domain, such as any described herein, such as a wild-type or a variant Phi29 polymerase domain, and any one or more of the polypeptide domains and/or tags, such as any described herein or any combination thereof. In some aspects, the fusion protein comprising a Phi29 polymerase domain and one or more polypeptide domains (e.g., one or more protein-protein interaction domains or one or more heterologous nucleic acid binding domains) is not coupled to a surface (e.g., an array). In some aspects, the fusion protein comprising a Phi29 polymerase domain and one or more polypeptide domains is not coupled to a nanopore.

### a. Phi29 Polymerase Domain

In some aspects, the provided fusion protein comprises a Phi29 polymerase domain, such as the polymerase domain of a Phi29 polymerase. In some aspects, the Phi29 polymerase domain of the fusion protein comprises a domain from a wild-type Phi29 polymerase or a variant Phi29 polymerase

Phi29 polymerase is a mesophilic DNA polymerase derived from the Bacillus subtilis phage Phi29. Phi29 polymerase has both a 3′-5′ exonuclease activity and nucleic acid polymerization activity. In view of its unique structural features, Phi29 can be used for particular methods of amplifying and/or sequencing DNA and RNA, including rolling circle amplification (RCA), multiple displacement amplification (MDA), and pair-end and single-cell sequencing. Phi29 is particularly useful for RCA reactions, in situ analysis, single cell analysis and/or multiplexed analysis, for example, multiplex endogenous analyte detection and single-cell multiomic analyses (e.g., genomic, transcriptomic, and proteomic profiling). Such exemplary assays are important tools for understanding the molecular basis of the biology of cells and organisms, and also for assessment and/or treatment of diseases.

In some embodiments, the provided fusion protein comprises a wild-type Phi29 polymerase domain. In some embodiments, the wild-type Phi29 polymerase domain comprises a sequence that has at least at or about 90%, 95%, 96%, 97%, 98%, 99%, or 99.5% sequence identity to the sequence set forth in SEQ ID NO: 1, or a portion thereof. In some embodiments, the wild-type Phi29 polymerase domain comprises the sequence set forth in SEQ ID NO:1. In some embodiments, the wild-type Phi29 polymerase domain comprises a sequence that has at least at or about 90%, 95%, 96%, 97%, 98%, 99%, or 99.5% sequence identity to the sequence set forth in SEQ ID NO:3, or a portion thereof. In some embodiments, the wild-type Phi29 polymerase domain comprises the sequence set forth in SEQ ID NO:3. In some embodiments, the wild-type Phi29 polymerase domain comprises a sequence that has at least at or about 90%, 95%, 96%, 97%, 98%, 99%, or 99.5% sequence identity to the sequence set forth in SEQ ID NO:6, or a portion thereof. In some embodiments, the wild-type Phi29 polymerase domain comprises the sequence set forth in SEQ ID NO:6.

In some embodiments, the provided fusion protein comprises variant Phi29 polymerase domain. In some embodiments, the variant Phi29 polymerase domain comprises one or more amino acid modifications or variations (e.g., substitutions, insertions, deletions, and/or combinations thereof) and/or fusions of additional peptide or protein sequences (e.g., for immobilizing the polymerase domain on a surface or otherwise tagging the polymerase domain enzyme), compared to the sequence of the wild-type Phi29 polymerase domain. Amino acid sequence variants of a Phi29 polymerase domain may be prepared by introducing appropriate modifications into the nucleotide sequence encoding the wild-type Phi29 polymerase domain. Such modifications include, for example, deletions from and/or insertions into and/or substitutions of residues within the nucleotide sequences encoding the wild-type Phi29 polymerase domain. The variant Phi29 polymerase domain can comprise any combination of deletion, insertion, and substitutions.

In some embodiments, the variant Phi29 polymerase domain is a variant of a wild-type or exonuclease deficient Phi29 polymerase domain, e.g., as described in U.S. Pat. Nos. 5,001,050, 5,198,543, 5,576,204, and Meijer et al. (2001) Microbiology and Molecular Biology Reviews, 65(2):261-287, all of which are herein incorporated by reference in their entireties. In some embodiments, the variant Phi29 polymerase domain of the provided fusion protein comprises a variant Phi29 polymerase domain as described in, for example, in US20110165652, US20210355459, US20070196846, US20140234853, US20160237412, US20170159033, US20200115686, US20180305673, US20180362941, US20210395707, US20230058852, or US20230095295, all of which are herein incorporated by reference in their entireties.

In some embodiments, the Phi29 polymerase domain comprises one or more amino acid substitutions at one or more positions selected from among positions 131, 135, 142, 148, 224, 239, 250, 253, 306, 308, 375, 437, 466, 476, 484, 508, 510, 512, 515, 539, 570, and 571 corresponding to the positions of the sequence set forth in SEQ ID NO: 1. In some embodiments, the Phi29 polymerase domain comprises an amino acid substitutions at position 131, corresponding to the positions of the sequence set forth in SEQ ID NO:1. In some embodiments, the Phi29 polymerase domain comprises an amino acid substitutions at position 135, corresponding to the positions of the sequence set forth in SEQ ID NO:1. In some embodiments, the Phi29 polymerase domain comprises an amino acid substitutions at position 142, corresponding to the positions of the sequence set forth in SEQ ID NO:1. In some embodiments, the Phi29 polymerase domain comprises an amino acid substitutions at position 148, corresponding to the positions of the sequence set forth in SEQ ID NO:1. In some embodiments, the Phi29 polymerase domain comprises an amino acid substitutions at position 224, corresponding to the positions of the sequence set forth in SEQ ID NO:1. In some embodiments, the Phi29 polymerase domain comprises an amino acid substitutions at position 239, corresponding to the positions of the sequence set forth in SEQ ID NO:1. In some embodiments, the Phi29 polymerase domain comprises an amino acid substitutions at position 250, corresponding to the positions of the sequence set forth in SEQ ID NO:1. In some embodiments, the Phi29 polymerase domain comprises an amino acid substitutions at position 253, corresponding to the positions of the sequence set forth in SEQ ID NO:1. In some embodiments, the Phi29 polymerase domain comprises an amino acid substitutions at position 306, corresponding to the positions of the sequence set forth in SEQ ID NO:1. In some embodiments, the Phi29 polymerase domain comprises an amino acid substitutions at position 308, corresponding to the positions of the sequence set forth in SEQ ID NO:1. In some embodiments, the Phi29 polymerase domain comprises an amino acid substitutions at position 375, corresponding to the positions of the sequence set forth in SEQ ID NO:1. In some embodiments, the Phi29 polymerase domain comprises an amino acid substitutions at position 437, corresponding to the positions of the sequence set forth in SEQ ID NO:1. In some embodiments, the Phi29 polymerase domain comprises an amino acid substitutions at position 466, corresponding to the positions of the sequence set forth in SEQ ID NO:1. In some embodiments, the Phi29 polymerase domain comprises an amino acid substitutions at position 476, corresponding to the positions of the sequence set forth in SEQ ID NO:1. In some embodiments, the Phi29 polymerase domain comprises an amino acid substitutions at position 484, corresponding to the positions of the sequence set forth in SEQ ID NO:1. In some embodiments, the Phi29 polymerase domain comprises an amino acid substitutions at position 508, corresponding to the positions of the sequence set forth in SEQ ID NO:1. In some embodiments, the Phi29 polymerase domain comprises an amino acid substitutions at position 510, corresponding to the positions of the sequence set forth in SEQ ID NO:1. In some embodiments, the Phi29 polymerase domain comprises an amino acid substitutions at position 512, corresponding to the positions of the sequence set forth in SEQ ID NO:1. In some embodiments, the Phi29 polymerase domain comprises an amino acid substitutions at position 515, corresponding to the positions of the sequence set forth in SEQ ID NO:1. In some embodiments, the Phi29 polymerase domain comprises an amino acid substitutions at position 539, corresponding to the positions of the sequence set forth in SEQ ID NO:1. In some embodiments, the Phi29 polymerase domain comprises one or more amino acid substitutions at one or more positions selected from among positions 570 and 571, corresponding to the positions of the sequence set forth in SEQ ID NO:1. In some embodiments, the Phi29 polymerase domain comprises an amino acid substitutions at position 570 and an amino acid substitution at position 571, corresponding to the positions of the sequence set forth in SEQ ID NO:1. In some embodiments, the Phi29 polymerase domain further comprises one or more additional amino acid substitutions other than at positions 570 and 571, corresponding to the positions of the sequence set forth in SEQ ID NO:1. In some embodiments, the Phi29 polymerase domain further comprises one or more amino acid substitutions at one or more positions selected from among positions 131, 135, 142, 148, 224, 239, 250, 253, 306, 308, 375, 437, 466, 476, 484, 508, 510, 512, 515, 539, in addition to positions 570 and 571, corresponding to the positions of the sequence set forth in SEQ ID NO:1. In some embodiments, the variant Phi29 polymerase domain comprises a D570 substitution, corresponding to the positions of the sequence set forth in SEQ ID NO:1. In some embodiments, the amino acid substitution at position 570 is D570S. In some embodiments, the variant Phi29 polymerase domain comprises a D570S substitution, corresponding to the positions of the sequence set forth in SEQ ID NO: 1. In some embodiments, the variant Phi29 polymerase domain comprises a T571 substitution, corresponding to the positions of the sequence set forth in SEQ ID NO:1. In some embodiments, the amino acid substitution at position 571 is T571V. In some embodiments, the variant Phi29 polymerase domain comprises a T571V substitution, corresponding to the positions of the sequence set forth in SEQ ID NO:1 In some embodiments, the Phi29 polymerase domain further comprises an N-terminal deletion of the methionine residue at position 1, corresponding to the positions of the sequence set forth in SEQ ID NO:1. In some embodiments, the Phi29 polymerase domain comprises an amino acid substitutions at position 570 and an amino acid substitution at position 571, corresponding to the positions of the sequence set forth in SEQ ID NO: 1. In some embodiments, the variant Phi29 polymerase domain comprises a D570S and a T571V substitution, corresponding to the positions of the sequence set forth in SEQ ID NO:1. In some embodiments, the Phi29 polymerase domain further comprises one or more amino acid substitutions at one or more positions selected from among positions 131, 135, 142, 148, 224, 239, 250, 253, 306, 308, 375, 437, 466, 476, 484, 508, 510, 512, 515, 539, in addition to amino acid substitutions D570S and a T571V, corresponding to the positions of the sequence set forth in SEQ ID NO:1.

In some embodiments, the Phi29 polymerase domain comprises a sequence that has at least at or about 90%, 95%, 96%, 97%, 98%, 99%, or 99.5% sequence identity to the sequence set forth in SEQ ID NO:51, or a portion thereof. In some embodiments, the Phi29 polymerase domain comprises the sequence set forth in SEQ ID NO:51. In some embodiments, the Phi29 polymerase domain comprises a sequence that has at least at or about 90%, 95%, 96%, 97%, 98%, 99%, or 99.5% sequence identity to the sequence set forth in SEQ ID NO:52, or a portion thereof. In some embodiments, the Phi29 polymerase domain comprises the sequence set forth in SEQ ID NO:52.

In some embodiments, the Phi29 polymerase domain comprises one or more amino acid substitutions at one or more positions selected from among positions M97, Y101, P129, S192, K305, R306, R308, L416, R496, Q497, T499, T534, K557, D570, T571, T573, and K575, corresponding to the positions of the sequence set forth in SEQ ID NO:1. In some embodiments, the Phi29 polymerase domain comprises one or more amino acid substitutions at one or more positions selected from among M97L, M97A, M97K, M97E, M97R, Y101F, P129R, S192A, K305R, R306Q, R306A, R306F, R306H, R306K, R306L, R306T, R308L, R308A, R308H, R308K, R308P, R308Q, L416A, L416E, L416H, L416I, L416S, L416T, L416Q, L416V, L416K, R496K, Q497R, Q497K, T499S, T499A, T499V, T499I, T499N, T534P, K557R, D570S, D570E, D570M, D570N, D570T, T571V, T571P, T573A, T573S, T573K, and K575R corresponding to the positions of the sequence set forth in SEQ ID NO:1. In some embodiments, the amino acid substitution at position 97 is selected from among M97L, M97A, M97K, M97E, and M97R, corresponding to the positions of the sequence set forth in SEQ ID NO:1. In some embodiments, the amino acid substitution at position 101 is Y101F, corresponding to the positions of the sequence set forth in SEQ ID NO: 1. In some embodiments, the amino acid substitution at position 129 is P129R, corresponding to the positions of the sequence set forth in SEQ ID NO:1. In some embodiments, the amino acid substitution at position 192 is S192A, corresponding to the positions of the sequence set forth in SEQ ID NO: 1. In some embodiments, the amino acid substitution at position 305 is K305R, corresponding to the positions of the sequence set forth in SEQ ID NO:1. In some embodiments, the amino acid substitution at position 306 is selected from among R306Q, R306A, R306F, R306H, R306K, R306L, and R306T, corresponding to the positions of the sequence set forth in SEQ ID NO: 1. In some embodiments, the amino acid substitution at position 308 is selected from among R308L, R308A, R308H, R308K, R308P, and R308Q, corresponding to the positions of the sequence set forth in SEQ ID NO:1. In some embodiments, the amino acid substitution at position 416 is selected from among L416A, L416E, L416H, L416I, L416S, L416T, L416Q, L416V, and L416K, corresponding to the positions of the sequence set forth in SEQ ID NO:1. In some embodiments, the amino acid substitution at position 496 is R496K, corresponding to the positions of the sequence set forth in SEQ ID NO:1. In some embodiments, the amino acid substitution at position 497 is selected from among Q497R and Q497K, corresponding to the positions of the sequence set forth in SEQ ID NO:1. In some embodiments, the amino acid substitution at position 499 is selected from among T499S, T499A, T499V, T4991, and T499N, corresponding to the positions of the sequence set forth in SEQ ID NO:1. In some embodiments, the amino acid substitution at position 534 is T534P, corresponding to the positions of the sequence set forth in SEQ ID NO: 1. In some embodiments, the amino acid substitution at position 557 is K557R, corresponding to the positions of the sequence set forth in SEQ ID NO:1. In some embodiments, the amino acid substitution at position 570 is selected from among D570S, D570E, D570M, D570N, and D570T, corresponding to the positions of the sequence set forth in SEQ ID NO:1. In some embodiments, the amino acid substitution at position 571 is selected from among T571V and T571P, corresponding to the positions of the sequence set forth in SEQ ID NO:1. In some embodiments, the amino acid substitution at position 573 is selected from among T573A, T573S, and T573K, corresponding to the positions of the sequence set forth in SEQ ID NO:1. In some embodiments, the amino acid substitution at position 575 is K575R, corresponding to the positions of the sequence set forth in SEQ ID NO:1.

In some embodiments, the Phi29 polymerase domain comprises one or more amino acid substitutions at one or more positions selected from among positions 83, 99, 131, 148, 420, and 570, corresponding to the positions of the sequence set forth in SEQ ID NO:1. In some embodiments, the Phi29 polymerase domain comprises an amino acid substitutions at position 83 and an amino acid substitution at position 420, corresponding to the positions of the sequence set forth in SEQ ID NO:1. In some embodiments, the Phi29 polymerase domain further comprises one or more amino acid substitutions other than at positions 83 and 420, corresponding to the positions of the sequence set forth in SEQ ID NO:1. In some embodiments, the amino acid substitution at position A83 is A83E or A83D. In some embodiments, the amino acid substitution at position E420 is E420R or E420K. In some embodiments, the variant Phi29 polymerase domain comprises an A83E substitution, corresponding to the positions of the sequence set forth in SEQ ID NO:1. In some embodiments, the variant Phi29 polymerase domain comprises an A83D substitution, corresponding to the positions of the sequence set forth in SEQ ID NO: 1. In some embodiments, the variant Phi29 polymerase domain comprises an E420R substitution, corresponding to the positions of the sequence set forth in SEQ ID NO:1. In some embodiments, the variant Phi29 polymerase domain comprises an E420K substitution, corresponding to the positions of the sequence set forth in SEQ ID NO:1. In some embodiments, the Phi29 polymerase domain further comprises an N-terminal deletion of the methionine residue at position 1, corresponding to the positions of the sequence set forth in SEQ ID NO:1. In some embodiments, the Phi29 polymerase domain comprises an amino acid substitutions at position 83 and an amino acid substitution at position 420, corresponding to the positions of the sequence set forth in SEQ ID NO: 1. In some embodiments, the variant Phi29 polymerase domain comprises an A83E substitution and an E420R substitution, corresponding to the positions of the sequence set forth in SEQ ID NO:1. In some embodiments, the variant Phi29 polymerase domain comprises an A83D substitution and an E420R substitution, corresponding to the positions of the sequence set forth in SEQ ID NO: 1. In some embodiments, the variant Phi29 polymerase domain comprises an A83E substitution and an E420K substitution, corresponding to the positions of the sequence set forth in SEQ ID NO:1. In some embodiments, the variant Phi29 polymerase domain comprises an A83D substitution and an E420K substitution, corresponding to the positions of the sequence set forth in SEQ ID NO:1.

In some embodiments, the Phi29 polymerase domain comprises one or more amino acid substitutions at one or more positions selected from among positions 99, 131, 148, and 570, corresponding to the positions of the sequence set forth in SEQ ID NO:1. In some embodiments, the Phi29 polymerase domain comprises one or more amino acid substitutions at one or more positions selected from among Q99I, K131E, Y1481, D570E, D570S, D570T, D570M, D570V, D570W, D570G, and D570C, corresponding to the positions of the sequence set forth in SEQ ID NO: 1. In some embodiments, the amino acid substitution at position 99 is Q991, corresponding to the positions of the sequence set forth in SEQ ID NO: 1. In some embodiments, the amino acid substitution at position 131 is K131E, corresponding to the positions of the sequence set forth in SEQ ID NO: 1. In some embodiments, the amino acid substitution at position 148 is Y148I, corresponding to the positions of the sequence set forth in SEQ ID NO: 1. In some embodiments, the amino acid substitution at position 570 is selected from among D570E, D570S, D570T, D570M, D570V, D570W, D570G, and D570C, corresponding to the positions of the sequence set forth in SEQ ID NO:1.

In some embodiments, the Phi29 polymerase domain comprises a sequence that has at least at or about 90%, 95%, 96%, 97%, 98%, 99%, or 99.5% sequence identity to the sequence set forth in SEQ ID NO: 51. In some embodiments, the Phi29 polymerase domain comprises the sequence of SEQ ID NO: 51. In some embodiments, the Phi29 polymerase domain comprises a sequence that has at least at or about 90%, 95%, 96%, 97%, 98%, 99%, or 99.5% sequence identity to the sequence set forth in SEQ ID NO: 51. In some embodiments, the fusion protein comprises a sequence that has at least at or about 90%, 95%, 96%, 97%, 98%, 99%, or 99.5% sequence identity to the sequence set forth in SEQ ID NO: 52. In some embodiments, the fusion protein comprises the sequence of SEQ ID NO: 52.

In some embodiments, the Phi29 polymerase domain comprises one or more amino acid substitutions at one or more positions selected from among positions 250, 253, 368, 437, 484, 515, and F526, corresponding to the positions of the sequence set forth in SEQ ID NO:1. In some embodiments, the Phi29 polymerase domain comprises one or more amino acid substitutions at one or more positions selected from among V2501, L253H, T368V, A437G, A484E, E515Q, E515K, and F526L, corresponding to the positions of the sequence set forth in SEQ ID NO:1. In some embodiments, the amino acid substitution at position 250 is V250I, corresponding to the positions of the sequence set forth in SEQ ID NO:1. In some embodiments, the amino acid substitution at position 253 is L253H, corresponding to the positions of the sequence set forth in SEQ ID NO:1. In some embodiments, the amino acid substitution at position 368 is T368V, corresponding to the positions of the sequence set forth in SEQ ID NO:1. In some embodiments, the amino acid substitution at position 437 is A437G, corresponding to the positions of the sequence set forth in SEQ ID NO:1. In some embodiments, the amino acid substitution at position 484 is A484E, corresponding to the positions of the sequence set forth in SEQ ID NO:1. In some embodiments, the amino acid substitution at position 526 is F526L, corresponding to the positions of the sequence set forth in SEQ ID NO:1. In some embodiments, the amino acid substitution at position 515 is E515Q or E515K, corresponding to the positions of the sequence set forth in SEQ ID NO:1. In some embodiments, the Phi29 polymerase domain further comprises an N-terminal deletion of the methionine residue at position 1, corresponding to the positions of the sequence set forth in SEQ ID NO:1. In some embodiments, the Phi29 polymerase domain comprises an amino acid substitutions at position 250 and an amino acid substitution at position 253, corresponding to the positions of the sequence set forth in SEQ ID NO:1. In some embodiments, the Phi29 polymerase domain further comprises one or more amino acid substitutions other than at positions 250 and 253, corresponding to the positions of the sequence set forth in SEQ ID NO: 1. In some embodiments, the variant Phi29 polymerase domain comprises a V250I substitution and a L253H substitution, corresponding to the positions of the sequence set forth in SEQ ID NO: 1. In some embodiments, the variant Phi29 polymerase domain comprises a V250I substitution and a L253H substitution in combination with one or more amino acid substitutions at one or more positions selected from among A437G, T368V, A484E, E515Q, E515K, and F526L.

In some embodiments, the Phi29 polymerase domain comprises one or more amino acid substitutions at one or more positions selected from among positions 68 and 83, corresponding to the positions of the sequence set forth in SEQ ID NO: 1. In some embodiments, the Phi29 polymerase domain comprises an amino acid substitutions at position 68 and an amino acid substitution at position 83, corresponding to the positions of the sequence set forth in SEQ ID NO:1. In some embodiments, the Phi29 polymerase domain further comprises one or more amino acid substitutions other than at positions 68 and 83, corresponding to the positions of the sequence set forth in SEQ ID NO:1. In some embodiments, the amino acid substitution at position 68 is selected from among A68S, A68C, A68N, A68P, A68T, A68W, and A68Y. In some embodiments, the amino acid substitution at position 68 is A68S. In some embodiments, the variant Phi29 polymerase domain comprises an A68S substitution, corresponding to the positions of the sequence set forth in SEQ ID NO: 1. In some embodiments, the amino acid substitution at position 83 is selected from among A83E, A83D, A83G, A83K, A83Q, and A83V. In some embodiments, the amino acid substitution at position 83 is A83E. In some embodiments, the variant Phi29 polymerase domain comprises an A83E substitution, corresponding to the positions of the sequence set forth in SEQ ID NO:1. In some embodiments, the Phi29 polymerase domain further comprises an N-terminal deletion of the methionine residue at position 1, corresponding to the positions of the sequence set forth in SEQ ID NO:1. In some embodiments, the Phi29 polymerase domain comprises an amino acid substitutions at position 68 and an amino acid substitution at position 83, corresponding to the positions of the sequence set forth in SEQ ID NO:1. In some embodiments, the variant Phi29 polymerase domain comprises an A68S substitution and an A83E substitution, corresponding to the positions of the sequence set forth in SEQ ID NO:1.

In some embodiments, the Phi29 polymerase domain comprises a sequence that has at least at or about 90%, 95%, 96%, 97%, 98%, 99%, or 99.5% sequence identity to the sequence set forth in SEQ ID NO:7, or a portion thereof. In some embodiments, the Phi29 polymerase domain comprises the sequence set forth in SEQ ID NO:7.

### A. Protein-Protein Interaction Domains

Provided herein is a fusion protein comprising a Phi29 polymerase and one or more protein-protein interaction domains. In some aspects, the provided fusion protein comprises one or more protein-protein interaction domains. In some aspects, the protein-protein interaction domain is or comprises one or more dimerization domain. In some embodiments, the fusion protein comprises a Phi29 polymerase domain and a dimerization domain of a different protein. In some aspects, the protein-protein interaction domain is or comprises one domain of a pair of protein coupling domains. In some aspects, the one or more protein-protein interaction domains is located N-terminal and/or C-terminal of the Phi29 polymerase domain. In some aspects, the protein-protein interaction domain is located N-terminal of the Phi29 polymerase domain. In some aspects, the protein-protein interaction domain is located C-terminal of the Phi29 polymerase domain.

In some embodiments, the protein-protein interaction comprises a transient interaction. In some embodiments, the protein-protein interaction comprises a stable interaction. In some embodiments, the protein-protein interaction is a non-covalent interaction. In some embodiments, the protein-protein interaction is a covalent interaction. In some embodiments, when a first fusion protein molecule and a second fusion protein molecule are present in proximity, the protein-protein interaction domains, such as the protein coupling domains, interact to produce a dimer comprising the first fusion protein molecule and the second fusion protein.

### 1. Dimerization Domain

In some aspects, the protein-protein interaction domain of the provided fusion protein comprises a dimerization domain. In some aspects, a dimerization domain is or comprises a polypeptide domain of a protein that allows protein monomers to form a macromolecular complex such as a dimer or a multimer, for example by non-covalent or covalent interactions. In some aspects, the dimerization domain comprises a dimerization domain from a protein that can form a dimer or a multimer in solution. In some aspects, the dimerization domain is an inducible dimerization domain. In some aspects, the fusion protein comprises a Phi29 polymerase and a dimerization domain of a different protein. In some aspects, the protein-protein interaction domain is located C-terminal of the Phi29 polymerase domain.

In some aspects, the dimerization domain comprises a kinesin 1 dimerization domain. Kinesin 1, also known as Kinesin-like protein KIF1A, is a motor protein involved in the transport of synaptic-vesicle precursors along axons. Kinesin 1 comprises a catalytic domain that aids its processive motility mechanisms. Kinesin 1 is a monomer but can interact with other kinesin and non-kinesin polypeptides. The coiled coil domain of Kinesin 1 facilitates dimerization of a fusion protein that comprises the dimerization domain. In some aspects, the kinesin 1 dimerization domain also provides improved solubility of a fusion protein that comprises the dimerization domain.

In some embodiments, the provided fusion protein comprises a dimerization domain from a kinesin 1 protein. In some embodiments, the provided fusion protein comprises a kinesin 1 dimerization protein. In some embodiments, the one or more dimerization domains comprise a kinesin 1 domain. In some embodiments, the kinesin 1 dimerization domain comprises a sequence that has at least at or about 90%, 95%, 96%, 97%, 98%, 99%, or 99.5% sequence identity to the sequence set forth in SEQ ID NO: 18, or a portion thereof. In some embodiments, the kinesin 1 dimerization domain comprises the sequence set forth in SEQ ID NO:18. In some embodiments, the kinesin 1 dimerization domain comprises a sequence that has at least at or about 90%, 95%, 96%, 97%, 98%, 99%, or 99.5% sequence identity to the sequence set forth in SEQ ID NO:20, or a portion thereof. In some embodiments, the kinesin 1 dimerization domain comprises the sequence set forth in SEQ ID NO:20.

In some embodiments, the kinesin 1 domain further comprises a kinesin 1 delta-tail domain. In some embodiments, the kinesin 1 delta-tail domain comprises a sequence that has at least at or about 90%, 95%, 96%, 97%, 98%, 99%, or 99.5% sequence identity to the sequence set forth in SEQ ID NO:21, or a portion thereof. In some embodiments, the kinesin 1 delta-tail domain comprises the sequence set forth in SEQ ID NO:21. In some embodiments, the kinesin 1 delta-tail domain is located at the C-terminal of the kinesin 1 domain. In some embodiments, the kinesin 1/kinesin 1 delta-tail domain is located at the N-terminal and/or C-terminal of the Phi29 polymerase domain.

In some embodiments, the dimerization domain is an inducible dimerization domain. In some embodiments, the dimerization domain can induce dimerization of fusion protein(s) comprising such domains, upon binding of an inducer. In some embodiments, exemplary dimerization domains include those described in, for example, Dang et al., Front Chem. 2022; 10: 829312, Bachmann et al., J Pept Sci. 2017 July; 23(7-8):624-630, all of which are herein incorporated by reference in their entireties. In some embodiments, exemplary dimerization domains include a dimerization domain from FKBP, a cyclophilin receptor, a steroid receptor, a tetracycline receptor, an estrogen receptor, a glucocorticoid receptor, a vitamin D receptor, Calcineurin A, CyP-Fas, FRB domain of mTOR, GyrB, GAI, GID1, Snap-tag and/or HaloTag, or portions or derivatives thereof. In some embodiments, the dimerization domain is an FK506 binding protein (FKBP) or derivative thereof, or fragment and/or multimer thereof, such as FKBP12v36.

### 2. Protein Coupling Domains

In some aspects, the protein-protein interaction domain of the provided fusion protein comprises a protein coupling domain. In some aspects, the protein-protein interaction domain comprises one partner domain of a pair of protein coupling domains. In some aspects, the protein-protein interaction domain of the provided fusion protein comprises a partner domain of a pair of protein coupling domains. In some aspects, the pair of partner domains together in proximity results in coupling of the two partner domains or two fusion protein molecules each comprising one of the two partner domains. In some aspects, when two different fusion proteins, each comprising one of the two partner domains of a pair of protein coupling domains are in proximity, the partner domains result in coupling of the two different fusion proteins, in some cases by a covalent interaction.

In some aspects, exemplary protein coupling domains include the SpyCatcher domain and the SpyTag domain, which are two partner domains of a pair of protein coupling domains. The SpyCatcher and SpyTag domains described herein comprise a protein coupling tool ideal for binding, labeling, immobilizing, and creating new protein architectures. When in proximity, the SpyTag domain spontaneously binds SpyCatcher domain through an amide bond resulting in a stable, irreversible bond. In some aspects, the SpyCatcher contains a cysteine residue for labeling with a dye or attachment to surfaces (e.g., beads). In some aspects, the SpyTag domain reacts with the SpyCatcher domain under a wide range of conditions and reactions. In some embodiments, the one or more protein-protein interaction domains comprise a SpyCatcher domain. In some embodiments, the one or more protein-protein interaction domains comprise a Spy Tag domain.

In some embodiments, the provided fusion protein comprises a SpyCatcher domain. In some embodiments, the SpyCatcher domain comprises a sequence that has at least at or about 90%, 95%, 96%, 97%, 98%, 99%, or 99.5% sequence identity to the sequence set forth in SEQ ID NO:22., or a portion thereof in some embodiments, the SpyCatcher domain comprises the sequence set forth in SEQ ID NO:22.

In some embodiments, the provided fusion protein comprises a SpyTag domain. In some embodiments, the Spy Tag domain comprises a sequence that has at least at or about 90%, 95%, 96%, 97%, 98%, 99%, or 99.5% sequence identity to the sequence set forth in SEQ ID NO:24, or a portion thereof. In some embodiments, the SpyTag domain comprises the sequence set forth in SEQ ID NO:24.

In some embodiments, when a first Phi29 fusion protein molecule comprising a SpyCatcher domain and a second Phi29 fusion protein molecule comprising a Spy Tag domain are present in proximity, the SpyCatcher domain and the SpyTag domain interact (e.g., protein coupling) to form a covalent bond to produce a dimer comprising the first Phi29 fusion protein molecule and the second Phi29 fusion protein molecule.

### B. Nucleic Acid Binding Domains

Provided herein is a fusion protein comprising a Phi29 polymerase domain and one or more heterologous nucleic acid binding domains. In some aspects, heterologous nucleic acid binding domains include domains from nucleic acid binding proteins that bind to nucleic acids, such as DNA or RNA, in a non-sequence-specific manner.

In some embodiments, the one or more heterologous nucleic acid binding domains are selected from among a HU domain, a helix-hairpin-helix (HhH) DNA binding motif, a helix-hairpin-helix fusion [(HhH)₂] DNA binding motif, a single stranded DNA binding domain, an SD07 DNA binding motif, an SS07 DNA binding motif. In some embodiments, the one or more heterologous nucleic acid binding domains comprises a HU domain. In some embodiments, the HU domain comprises a bacterial HU domain. In some embodiments, the HU domain comprises an E. coli HU domain, optionally a HU domain from a HuB, a HuC or a HuD protein.

In some aspects, HU is a small, basic, heat-stable, histone-like DNA-binding protein derived from bacteria. Members of the HU family include HuR, HuB, HuC and HuD, some which also have homologs in animals. HU can bind non-specifically to regions of DNA, and can recognize defined structures common to both DNA and RNA. In some aspects, a fusion protein comprising a Phi29 polymerase domain and a HU domain provides advantages including enhancing processivity and improving affinity for DNA hybridized to primer.

In some embodiments, the provided fusion protein comprises a HU domain. In some embodiments, the HU domain comprises a bacterial HU domain. In some embodiments, the HU domain comprises an E. coli HU domain. In some embodiments, the HU domain is from a HuA/HuR, a HuB, a HuC or a HuD protein. In some embodiments, the HU domain comprises a sequence that has at least at or about 90%, 95%, 96%, 97%, 98%, 99%, or 99.5% sequence identity to the sequence set forth in SEQ ID NO:26, or a portion thereof. In some embodiments, the HU domain comprises the sequence set forth in SEQ ID NO:26.

In some aspects, the heterologous nucleic acid binding domain is an HhH domain. In some aspects, exemplary heterologous nucleic acid binding domains include those described in, for example, Ordonez et al., Sci Rep. 2020; 10:15046 and Pavlov et al., PNAS 2002; 99(21): 13510-5, all of which are herein incorporated by reference in their entireties. In some aspects, exemplary heterologous nucleic acid binding domain includes HhH domains or motifs from any of the following proteins: Accession No. Q93H64 from Streptomyces avermitilis; Accession No. Q9CEH6 from Lactococcus lactis; Accession No. 000847 from Trypanosoma brucei; Accession No. Q8Y7P2 from Listeria monocytogenes; Accession No. RL32_HALNI from Halobacterium; Accession No. Q99YR8 from Streptococcus pyogenes; Accession No. Q55769 from Synechocystis; Accession No. YACK_BACSU from Bacillus subtilis; Accession No. Q91VP3 from Mus musculus; Accession No. P91153 from Caenorhabditis elegans; Accession No. P77987 from Thermus aquaticus; Accession No. Q9KGGO from Bacillus halodurans; Accession No. AAM31590 from Methanosarcina mazei goel; Accession No. Q9APNO from Myxococcus xanthus; Accession No. Q8XHQ0 from Clostridium perfringens; Accession No. Q9COF7 from Homo sapiens; Accession No. Q8XGVO from Salmonella typhimurium; Accession No. 026650 from Methanobacterium thermoautotrophicum; Accession No. AAM02649 from Methanopyrus kandleri av19; Accession No. Q9D7J3 from Mus musculus; Accession No. AAM38234 from Xanthomonas axonopodis; Accession No. P94624 from Clostridium difficile; Accession No. 074103 from Pyrococcus horikoshii; Accession No. AAK99660 from Streptococcus pneumoniae r6; Accession No. AAM02248 from Methanopyrus kandleri av19; Accession No. AAH28155 from Homo sapiens; Accession No. P71728 from Mycobacterium tuberculosis; Accession No. AAM06574 from Methanosarcina acetivorans str; Accession No. Q9YC15 from Aeropyrum pernix; Accession No. AAK99347 from Streptococcus pneumoniae r6; Accession No. AAL93832 from nucleatum; Accession No. AAH08930 from Homo sapiens; Accession No. EX9_ECOLI from Escherichia coli; Accession No. AAL81517 from Pyrococcus furiosus dsm 3638; Accession No. Q8Y3B2 from Ralstonia solanacearum; Accession No. Q8XBD5 from Escherichia coli 0157:h7; Accession No. Q8XIJ9 from Clostridium perfringens; Accession No. Q9PJAO from Campylobacter jejuni; Accession No. Q9RA49 from Thermus aquaticus; Accession No. AAM25145 from Thermoanaerobacter tengcongensis; Accession No. BAB95406 from Staphylococcus aureus; Accession No. AAM01328 from Methanopyrus kandleri av19; Accession No. AAM25645 from Thermoanaerobacter tengcongensis; Accession No. AAL95150 from nucleatum; Accession No. Q8VQ77 from Bacillus licheniformis; Accession No. HELS METJA from Methanococcus jannaschii; Accession No. Q92D76 from Listeria innocua; Accession No. AAL95286 from nucleatum; Accession No. Q8XBL5 from Escherichia coli 0157:h7; Accession No. Q9HZ87 from Pseudomonas aeruginosa; Accession No. Q9GRA1 from Hydra oligactis; Accession No. Q98AC3 from Rhizobium loti; Accession No. RPOA_SPIMX from Spirogyra maxima; Accession No. AAL91594 from Homo sapiens; Accession No. Q8Y8F9 from Listeria monocytogenes; Accession No. Q99LC7 from Mus musculus; Accession No. P72464 from Streptomyces lividans; Accession No. Q96K64 from Homo sapiens; Accession No. Q974S1 from Sulfolobus tokodaii; Accession No. Q9HMU5 from Halobacterium; Accession No. Q8XE54 from Escherichia coli 0157:h7; Accession No. Q96NY9 from Homo sapiens; Accession No. AAM34796 from Homo sapiens; Accession No. AAL98145 from Streptococcus pyogenes mgas8232; Accession No. 027790 from Methanobacterium thermoautotrophicum; Accession No. AAL92998 from Dictyostelium discoideum; Accession No. AAM05700 from Methanosarcina acetivorans str; Accession No. RADA METMP from Methanococcus maripaludis; Accession No. AAK99292 from Streptococcus pneumoniae r6; Accession No. AAM04167 from Methanosarcina acetivorans str; Accession No. Q91869 from Xenopus laevis; Accession No. AAM06907 from Methanosarcina acetivorans str; Accession No. Q94K65 from Arabidopsis thaliana; Accession No. Q8XIE6 from Clostridium perfringens; Accession No. AAH27384 from Mus musculus; Accession No. Q97EC6 from Clostridium acetobutylicum; Accession No. AAM23354 from Thermoanaerobacter tengcongensis; Accession No. Q9FF89 from Arabidopsis thaliana; Accession No. AAM02891 from Methanopyrus kandleri av19; Accession No. HELS_METTH from Methanobacterium thermoautotrophicum; Accession No. AAL82139 from Pyrococcus furiosus dsm 3638; Accession No. 030253 from Archaeoglobus fulgidus; Accession No. Q9HSD6 from Halobacterium; Accession No. Q9WY43 from Thermotoga maritima; Accession No. AAK84937 from Drosophila melanogaster; Accession No. AAL98006 from Streptococcus pyogenes mgas8232; Accession No. 085197 from Streptococcus pneumoniae; Accession No. AAM31083 from Methanosarcina mazei goel; Accession No. AAM24182 from Thermoanaerobacter tengcongensis; Accession No. Q9HMW5 from Halobacterium; Accession No. RECR_BACSU from Bacillus subtilis; Accession No. Q9BTN8 from Homo sapiens; Accession No. Q8X233 from Aeropyrum pernix; and Accession No. NOD_DROME from Drosophila melanogaster. In some aspects, the polypeptide domain comprises an [(HhH)₂] domain, for example, as described in Ordonez et al., Sci Rep. 2020; 10:15046, the content of which is herein incorporated by reference in its entirety. In some aspects, the polypeptide domain comprises an [(HhH)₂] domain comprising one or more of any of the HhH domains described herein. In some aspects, the polypeptide domain comprises an [(HhH)₂] domain comprising a fusion of any two or more of the HhH domains described herein. In some aspects, a [(HhH)₂] domain is a fusion of the H and I DNA-binding domains of Methanopyrus kandleri topoisomerase V.

### C. Other Components

In some embodiments, a fusion protein disclosed herein further comprises one or more heterologous tags and/or linkers.

In some embodiments, the fusion protein further comprises one or more tags. In some aspects, the tag is an affinity tag and/or a solubility tag. In some aspects, the tag is a tag for recombinant purification, substrate binding, or addition of other tags. Exemplary tags include purification, substrate binding, or other tags, such as a poly histidine (HIS) tag, a His10 tag, a His6 tag, an alanine tag, an Ala10 tag, an Ala16 tag, a biotin tag, a biotin ligase recognition sequence or other biotin attachment site (e.g., a BiTag or a Btag or variant thereof, e.g., BtagV1-11), a GST tag, an S Tag, a SNAP-tag, an hemagglutinin (HA) tag, a DSB (Sso7D) tag, a lysine tag, a NanoTag, a Cmyc tag. In some aspects, the tag is selected from among a poly histidine (HIS) tag, a solubility enhancement tag (SET), a small ubiquitin modified (SUMO) tag, a Fasciola hepatica 8-kDa antigen (Fh8) tag, a thioredoxin (Trx) tag, a glutathione-s-transferase (GST) tag, a maltose-binding protein (MBP) tag, an IgG domain B1 of protein G (GB1) tag, a mutated dehalogenase (Halo) tag, a steptavidin-binding peptide (SBP) tag, and a Tamavidin tag. In some aspects, exemplary tags include those described in, for example, Kimple et al., Curr Protoc Protein Sci. 2013; 73: Unit-9.9, the content of which is herein incorporated by reference in its entirety.

In some embodiments, the tag is selected from among a poly histidine (HIS) tag, a solubility enhancement tag (SET), a small ubiquitin modified (SUMO) tag, a Fasciola hepatica 8-kDa antigen (Fh8) tag, a thioredoxin (Trx) tag, a glutathione-s-transferase (GST) tag, a maltose-binding protein (MBP) tag, an IgG domain B1 of protein G (GB1) tag, a mutated dehalogenase (Halo) tag, a steptavidin-binding peptide (SBP) tag, and a Tamavidin tag.

In some embodiments, the tag is a poly histidine (HIS) tag. In some embodiments, the HIS tag comprises a sequence that has at least at or about 90%, 95%, 96%, 97%, 98%, 99%, or 99.5% sequence identity to the sequence set forth in SEQ ID NO:43, or a portion thereof. In some embodiments, the HIS tag comprises the sequence set forth in SEQ ID NO:43. In some embodiments, the HIS tag comprises a sequence that has at least at or about 90%, 95%, 96%, 97%, 98%, 99%, or 99.5% sequence identity to the sequence set forth in SEQ ID NO:24, or a portion thereof. In some embodiments, the HIS tag comprises the sequence set forth in SEQ ID NO:24.

In some embodiments, the tag is a SUMO tag. In some embodiments, the SUMO tag comprises a sequence that has at least at or about 90%, 95%, 96%, 97%, 98%, 99%, or 99.5% sequence identity to the sequence set forth in SEQ ID NO:41, or a portion thereof. In some embodiments, the SUMO tag comprises the sequence set forth in SEQ ID NO:41.

Exemplary tags may be included to improve the solubility of a Phi29 fusion protein, e.g., during recombinant expression, purification, and/or post-purification. In some aspects, the heterologous sequence comprises a tag for improving the Phi29 fusion protein solubility. In some aspects, the tag is a solubility enhancement tag (SET). In some aspects, the tag has a net negative charge at pH 7. In some aspects, the tag comprising a negative charge improves the Phi29 fusion protein solubility and/or stability. In some aspects, the tag has a net negative charge of at least or at least about −1, −2, −4, −6, −10, −12, −14, −15, −16, −17, or −18. In some aspects, the tag has a net negative charge of −1, −2, −4, −6, −10, −12, −14, −15, −16, −17, or −18. In some aspects, the tag comprises one or more acidic amino acid residue, for example, an aspartic acid (Asp, D) and/or a glutamic acid (Glu, E). In some aspects, the tag comprises at least 2, 3, 4, 5, 6, 7, 8, 9, 10, or more acidic amino acid residues. In some aspects, the tag is between 1 and 50, 2 and 40, 3 and 30, 5 and 20, 5 and 12, or 8 and 20 amino acid residues in length. In some embodiments, the tag is a SET tag. In some embodiments, the SET tag comprises a sequence that has at least at or about 90%, 95%, 96%, 97%, 98%, 99%, or 99.5% sequence identity to the sequence set forth in SEQ ID NO:46, or a portion thereof. In some embodiments, the SET tag comprises the sequence set forth in SEQ ID NO:46. In some embodiments, the SET tag comprises a sequence that has at least at or about 90%, 95%, 96%, 97%, 98%, 99%, or 99.5% sequence identity to the sequence set forth in SEQ ID NO:47, or a portion thereof. In some embodiments, the SET tag comprises the sequence set forth in SEQ ID NO:47.

In some aspects, the tag is directly fused to the Phi29 polymerase domain. In some aspects, the tag is fused directly to the N- and/or C-terminal end of the Phi29 polymerase domain, the protein-protein interaction domain (e.g., dimerization domain) and/or the nucleic acid binding domain. In some aspects, the tag is indirectly fused to the Phi29 polymerase domain. In some aspects, the tag is fused to a second sequence, for example, a linker or another tag. In some aspects, the tag is inserted in the interior portion of the Phi29 polymerase domain. In some aspects, the tag is indirectly fused to the protein-protein interaction domain, e.g., dimerization domain. In some aspects, the tag is inserted in the interior portion of the protein-protein interaction domain, e.g., dimerization domain. In some aspects, the tag is indirectly fused to the nucleic acid binding domain. In some aspects, the tag is inserted in the interior portion of the nucleic acid binding domain.

In some aspects, the fusion protein comprises one or more linkers. In some embodiments, the linker is at least at or about 1, 2, 3, 4, 5, 6, 7, 8, 9, 10, 15, 20, 25, 30, 40, or 50 amino acids in length. In some embodiments, the linker is a cleavable linker. In some embodiments, the linker is a non-cleavable linker. In some aspects, the fusion protein comprises two or more linkers. In some aspects, the linker comprises one or more glycine, serine, alanine, histidine, arginine, lysine, glutamine and/or proline residues. In some aspects, the linker comprises one or more glycine and/or serine residues. In some aspects, the linker is 1, 2, 3, 4, 5, 6, 7, 8, 9, 10, or more amino acid residues, or a range defined by any of the foregoing, in length. In some aspects, the linker is between 1 and 30, 2 and 25, 3 and 20, 4 and 18, 5 and 16, 6 and 14, 7 and 12, or 8 and 10 amino acid residues in length.

In some aspects, the linker is a flexible linker or a rigid linker. In some aspects, the linker comprises a cleavable linker sequence. Cleavable linkers are often cleaved, for example, by enzymatic or reductive mechanisms. Exemplary cleavable linkers include protease sensitive linkers, cyclopeptide linkers, and/or disulfide sensitive linkers. In some aspects, the fusion protein comprises a cleavable sequence. Cleavable sequences are often cleaved, for example, by enzymatic or reductive mechanisms. Exemplary cleavable sequences include protease sensitive sequences, cyclopeptide sequences, and/or disulfide sensitive sequences. In some aspects, the fusion protein comprises a linker comprising a cleavable sequence. In some aspects, the cleavable sequence is included in a linker. In some aspects, the cleavable sequence is included in a non-linker sequence. Exemplary cleavable sequences can be included to remove one or more fusion protein from the polymerase fusion protein, e.g., after recombinant expression and/or purification.

In some embodiments, the linker comprises a sequence selected from the sequence set forth in any of SEQ ID NOS:9-17 and 48. In some aspects, the linker comprises SEQ ID NO:9. In some aspects, the linker comprises SEQ ID NO:10. In some aspects, the linker comprises SEQ ID NO: 11. In some aspects, the linker comprises SEQ ID NO: 12. In some aspects, the linker comprises SEQ ID NO:13. In some aspects, the linker comprises SEQ ID NO:14. In some aspects, the linker comprises SEQ ID NO:15. In some aspects, the linker comprises SEQ ID NO:16. In some aspects, the linker comprises SEQ ID NO:17. In some aspects, the linker comprises SEQ ID NO:16. In some aspects, the linker comprises SEQ ID NO:48.

In some aspects, the linker is directly fused to the Phi29 polymerase domain. In some aspects, the linker is fused directly to the N- and/or C-terminal end of the Phi29 polymerase domain, the protein-protein interaction domain (e.g., dimerization domain) and/or the nucleic acid binding domain. In some aspects, the linker is indirectly fused to the Phi29 polymerase domain. In some aspects, the linker is fused to a second sequence, for example, a linker or another linker. In some aspects, the linker is inserted in the interior portion of the Phi29 polymerase domain. In some aspects, the linker is indirectly fused to the protein-protein interaction domain, e.g., dimerization domain. In some aspects, the linker is inserted in the interior portion of the protein-protein interaction domain, e.g., dimerization domain. In some aspects, the linker is indirectly fused to the nucleic acid binding domain. In some aspects, the linker is inserted in the interior portion of the nucleic acid binding domain.

### D. Exemplary Fusion Proteins

In some aspects, provided herein is a Phi29 fusion protein comprising a Phi29 polymerase and one or more kinesin 1 domains. In some embodiments, the Phi29 polymerase domain comprises a sequence that has at least at or about 90%, 95%, 96%, 97%, 98%, 99%, or 99.5% sequence identity to the sequence set forth in SEQ ID NO: 1 or 3, or a portion thereof; and the kinesin 1 domain comprises a sequence that has at least at or about 90%, 95%, 96%, 97%, 98%, 99%, or 99.5% sequence identity to the sequence set forth in SEQ ID NO: 18, or a portion thereof. In some embodiments, the Phi29 polymerase domain comprises the sequence set forth in SEQ ID NO:3; and the kinesin 1 domain comprises the sequence set forth in SEQ ID NO:18. In some embodiments, the fusion protein comprises an N-terminal HIS tag followed by a SUMO tag (N-His/SUMO). In some embodiments, the Phi29 polymerase domain comprises any one or more amino acid substitutions described in Section II.A herein. In some embodiments, the Phi29 polymerase domain comprises a D570S amino acid substitution, wherein the amino acid numbering is according the positions of the sequence set forth in SEQ ID NO:1.

In some embodiments, the Phi29 polymerase domain comprises a sequence that has at least at or about 90%, 95%, 96%, 97%, 98%, 99%, or 99.5% sequence identity to the sequence set forth in SEQ ID NO: 1 or 3, or a portion thereof; and the kinesin 1 domain comprises a sequence that has at least at or about 90%, 95%, 96%, 97%, 98%, 99%, or 99.5% sequence identity to the sequence set forth in SEQ ID NO:20, or a portion thereof. In some embodiments, the Phi29 polymerase domain comprises the sequence set forth in SEQ ID NO:3; and the kinesin 1 domain comprises the sequence set forth in SEQ ID NO:20. In some embodiments, the kinesin 1 domain is located at the N-terminal and/or C-terminal of the Phi29 polymerase domain. In some embodiments, the fusion protein comprises an N-terminal HIS tag followed by a SUMO tag (N-His/SUMO). In some embodiments, the Phi29 polymerase domain comprises any one or more amino acid substitutions described in Section II.A herein. In some embodiments, the Phi29 polymerase domain comprises a D570S amino acid substitution, wherein the amino acid numbering is according the positions of the sequence set forth in SEQ ID NO:1.

In some aspects, the fusion protein provided herein comprises from N-terminal to C-terminal a Phi29 polymerase domain, a linker, a kinesin 1 dimerization domain, a linker, and a tag. In some embodiments, the fusion protein provided herein comprises from N-terminal to C-terminal SEQ ID NO:3, SEQ ID NO:9, SEQ ID NO: 18, SEQ ID NO:48, and SEQ ID NO:43. In some embodiments, the fusion protein provided herein comprises from N-terminal to C-terminal SEQ ID NO:1, SEQ ID NO:9, SEQ ID NO: 18, SEQ ID NO:48, and SEQ ID NO:43. In some embodiments, the fusion protein provided herein comprises from N-terminal to C-terminal SEQ ID NO:6, SEQ ID NO:9, SEQ ID NO: 18, SEQ ID NO:48, and SEQ ID NO:43. In some embodiments, the fusion protein provided herein comprises from N-terminal to C-terminal SEQ ID NO:3, SEQ ID NO:9, SEQ ID NO:20, SEQ ID NO:48, and SEQ ID NO:43. In some embodiments, the fusion protein provided herein comprises from N-terminal to C-terminal SEQ ID NO:1, SEQ ID NO:9, SEQ ID NO:20, SEQ ID NO:48, and SEQ ID NO:43. In some embodiments, the fusion protein provided herein comprises from N-terminal to C-terminal SEQ ID NO:6, SEQ ID NO:9, SEQ ID NO:20, SEQ ID NO:48, and SEQ ID NO:43. In some embodiments, the fusion protein provided herein comprises from N-terminal to C-terminal SEQ ID NO:51, SEQ ID NO:9, SEQ ID NO:20, SEQ ID NO:48, and SEQ ID NO:43. In some embodiments, the Phi29 polymerase domain comprises any one or more amino acid substitutions described in Section II.A herein. In some embodiments, the Phi29 polymerase domain comprises a D570S amino acid substitution, wherein the amino acid numbering is according the positions of the sequence set forth in SEQ ID NO:1.

In some aspects, the fusion protein provided herein comprises from N-terminal to C-terminal a variant Phi29 polymerase domain, a linker, a kinesin 1 dimerization domain, a linker, and a tag. In some embodiments, the fusion protein provided herein comprises from N-terminal to C-terminal SEQ ID NO:7, SEQ ID NO:9, SEQ ID NO: 18, SEQ ID NO:48, and SEQ ID NO:43. In some embodiments, the fusion protein provided herein comprises from N-terminal to C-terminal SEQ ID NO:7, SEQ ID NO:9, SEQ ID NO:20, SEQ ID NO:48, and SEQ ID NO:43. In some embodiments, the Phi29 polymerase domain comprises any one or more amino acid substitutions described in Section II.A herein. In some embodiments, the Phi29 polymerase domain comprises a D570S amino acid substitution, wherein the amino acid numbering is according the positions of the sequence set forth in SEQ ID NO:1.

In some embodiments, the fusion protein comprises a sequence that has at least at or about 90%, 95%, 96%, 97%, 98%, 99%, or 99.5% sequence identity to the sequence set forth in SEQ ID NO:28. In some embodiments, the fusion protein comprises the sequence set forth in SEQ ID NO:28.

In some embodiments, the fusion protein comprises a sequence that has at least at or about 90%, 95%, 96%, 97%, 98%, 99%, or 99.5% sequence identity to the sequence set forth in SEQ ID NO:30. In some embodiments, the fusion protein comprises the sequence set forth in SEQ ID NO:30.

In some embodiments, the fusion protein comprises a sequence that has at least at or about 90%, 95%, 96%, 97%, 98%, 99%, or 99.5% sequence identity to the sequence set forth in SEQ ID NO:49. In some embodiments, the fusion protein comprises the sequence set forth in SEQ ID NO:49.

In some embodiments, the fusion protein comprises a sequence that has at least at or about 90%, 95%, 96%, 97%, 98%, 99%, or 99.5% sequence identity to the sequence set forth in SEQ ID NO:31. In some embodiments, the fusion protein comprises the sequence set forth in SEQ ID NO:31.

In some embodiments, the fusion protein comprises a sequence that has at least at or about 90%, 95%, 96%, 97%, 98%, 99%, or 99.5% sequence identity to the sequence set forth in SEQ ID NO:32. In some embodiments, the fusion protein comprises the sequence set forth in SEQ ID NO:32.

In some embodiments, the fusion protein comprises a sequence that has at least at or about 90%, 95%, 96%, 97%, 98%, 99%, or 99.5% sequence identity to the sequence set forth in SEQ ID NO:33. In some embodiments, the fusion protein comprises the sequence set forth in SEQ ID NO:33.

In some embodiments, the fusion protein comprises a sequence that has at least at or about 90%, 95%, 96%, 97%, 98%, 99%, or 99.5% sequence identity to the sequence set forth in SEQ ID NO:51. In some embodiments, the fusion protein comprises the sequence set forth in SEQ ID NO:51. In some embodiments, the fusion protein comprises a sequence that has at least at or about 90%, 95%, 96%, 97%, 98%, 99%, or 99.5% sequence identity to the sequence set forth in SEQ ID NO:52. In some embodiments, the fusion protein comprises the sequence set forth in SEQ ID NO:52.

In some aspects, provided herein is a Phi29 fusion protein comprising a Phi29 polymerase and one or more SpyCatcher domains. In some embodiments, the Phi29 polymerase domain comprises a sequence that has at least at or about 90%, 95%, 96%, 97%, 98%, 99%, or 99.5% sequence identity to the sequence set forth in SEQ ID NO: 1 or 3, or a portion thereof; and the SpyCatcher domain comprises a sequence that has at least at or about 90%, 95%, 96%, 97%, 98%, 99%, or 99.5% sequence identity to the sequence set forth in SEQ ID NO:22, or a portion thereof. In some embodiments, the Phi29 polymerase domain comprises the sequence set forth in SEQ ID NO:3; and the SpyCatcher domain comprises the sequence set forth in SEQ ID NO:22. In some embodiments, the SpyCatcher domain is located at the N-terminal and/or C-terminal of the Phi29 polymerase domain. In some embodiments, the SpyCatcher domain is located at the C-terminal of the Phi29 polymerase domain. In some embodiments, the fusion protein comprises an N-terminal HIS tag followed by a SUMO tag (N-His/SUMO).

In some aspects, the fusion protein provided herein comprises from N-terminal to C-terminal a Phi29 polymerase domain, a linker, and a SpyCatcher domain. In some embodiments, the fusion protein provided herein comprises from N-terminal to C-terminal SEQ ID NO:3, SEQ ID NO:10, and SEQ ID NO:22. In some embodiments, the fusion protein provided herein comprises from N-terminal to C-terminal SEQ ID NO:1, SEQ ID NO:10, and SEQ ID NO:22. In some embodiments, the fusion protein provided herein comprises from N-terminal to C-terminal SEQ ID NO:6, SEQ ID NO:10, and SEQ ID NO:22. In some embodiments, the fusion protein comprises an N-terminal HIS tag followed by a SUMO tag (N-His/SUMO).

In some embodiments, the fusion protein comprises a sequence that has at least at or about 90%, 95%, 96%, 97%, 98%, 99%, or 99.5% sequence identity to the sequence set forth in SEQ ID NO:34. In some embodiments, the fusion protein comprises the sequence set forth in SEQ ID NO:34. In some embodiments, the fusion protein comprises an N-terminal HIS tag followed by a SUMO tag (N-His/SUMO).

In some aspects, provided herein is a Phi29 fusion protein comprising a Phi29 polymerase domain and one or more SpyTag domains. In some embodiments, the Phi29 polymerase domain comprises a sequence that has at least at or about 90%, 95%, 96%, 97%, 98%, 99%, or 99.5% sequence identity to the sequence set forth in SEQ ID NO: 1 or 3, or a portion thereof; and the Spy Tag domain comprises a sequence that has at least at or about 90%, 95%, 96%, 97%, 98%, 99%, or 99.5% sequence identity to the sequence set forth in SEQ ID NO:24, or a portion thereof. In some embodiments, the Phi29 polymerase domain comprises the sequence set forth in SEQ ID NO:3; and the SpyTag domain comprises the sequence set forth in SEQ ID NO:24. In some embodiments, the SpyTag domain is located at the N-terminal and/or C-terminal of the Phi29 polymerase domain. In some embodiments, the SpyTag domain is located C-terminal to the Phi29 polymerase domain. In some embodiments, the fusion protein comprises an N-terminal HIS tag followed by a SUMO tag (N-His/SUMO).

In some aspects, the fusion protein provided herein comprises from N-terminal to C-terminal a Phi29 polymerase domain, a linker, a SpyTag domain, and a linker. In some embodiments, the fusion protein provided herein comprises from N-terminal to C-terminal SEQ ID NO:3, SEQ ID NO: 10, and SEQ ID NO:24. In some embodiments, the fusion protein provided herein comprises from N-terminal to C-terminal SEQ ID NO:1, SEQ ID NO: 10, and SEQ ID NO:24. In some embodiments, the fusion protein provided herein comprises from N-terminal to C-terminal SEQ ID NO:6, SEQ ID NO: 10, and SEQ ID NO:24. In some embodiments, the fusion protein comprises an N-terminal HIS tag followed by a SUMO tag (N-His/SUMO).

In some embodiments, the fusion protein comprises a sequence that has at least at or about 90%, 95%, 96%, 97%, 98%, 99%, or 99.5% sequence identity to the sequence set forth in SEQ ID NO:36. In some embodiments, the fusion protein comprises the sequence set forth in SEQ ID NO:36. In some embodiments, the fusion protein comprises an N-terminal HIS tag followed by a SUMO tag (N-His/SUMO).

In some aspects, provided herein is a Phi29 fusion protein comprising a Phi29 polymerase and one or more HU domains. In some embodiments, the Phi29 polymerase domain comprises a sequence that has at least at or about 90%, 95%, 96%, 97%, 98%, 99%, or 99.5% sequence identity to the sequence set forth in SEQ ID NO: 1 or 3, or a portion thereof; and the HU domain comprises a sequence that has at least at or about 95%, 96%, 97%, 98%, 99%, or 99.5% sequence identity to the sequence set forth in SEQ ID NO:26, or a portion thereof. In some embodiments, the Phi29 polymerase domain comprises the sequence set forth in SEQ ID NO:3; and the HU domain comprises the sequence set forth in SEQ ID NO:26. In some embodiments, the HU domain is located at the N-terminal and/or C-terminal of the Phi29 polymerase domain. In some embodiments, the fusion protein comprises an N-terminal HIS tag followed by a SUMO tag (N-His/SUMO).

In some aspects, the fusion protein provided herein comprises from N-terminal to C-terminal a Phi29 polymerase domain, a linker, and a HU domain. In some embodiments, the fusion protein provided herein comprises from N-terminal to C-terminal SEQ ID NO:3, SEQ ID NO:10, and SEQ ID NO:26. In some embodiments, the fusion protein provided herein comprises from N-terminal to C-terminal SEQ ID NO:1, SEQ ID NO:10, and SEQ ID NO:26. In some embodiments, the fusion protein provided herein comprises from N-terminal to C-terminal SEQ ID NO:6, SEQ ID NO:10, and SEQ ID NO:26. In some embodiments, the fusion protein comprises an N-terminal HIS tag followed by a SUMO tag (N-His/SUMO).

In some aspects, the fusion protein provided herein comprises from N-terminal to C-terminal a Phi29 polymerase domain, a linker, a HU domain, a linker, and a tag. In some embodiments, the fusion protein provided herein comprises from N-terminal to C-terminal SEQ ID NO:3, SEQ ID NO:9, SEQ ID NO:26, SEQ ID NO:48, and SEQ ID NO:43. In some embodiments, the fusion protein provided herein comprises from N-terminal to C-terminal SEQ ID NO: 1, SEQ ID NO:9, SEQ ID NO:26, SEQ ID NO:48, and SEQ ID NO:43. In some embodiments, the fusion protein provided herein comprises from N-terminal to C-terminal SEQ ID NO:6, SEQ ID NO:9, SEQ ID NO:26, SEQ ID NO:48, and SEQ ID NO:43. In some embodiments, the fusion protein comprises an N-terminal HIS tag followed by a SUMO tag (N-His/SUMO).

In some embodiments, the fusion protein comprises from N-terminal to C-terminal a poly histidine tag (HIS tag), a SUMO tag, a Phi29 polymerase domain, and a protein-protein interaction domain, optionally wherein the protein-protein interaction domain is connected to the polymerase domain via a linker. In some embodiments, the HIS tag comprises a sequence that has at least at or about 90%, 95%, 96%, 97%, 98%, 99%, or 99.5% sequence identity to the sequence set forth in SEQ ID NO:43, or a portion thereof. In some embodiments, the HIS tag comprises the sequence set forth in SEQ ID NO:43. In some embodiments, the HIS tag comprises a sequence that has at least at or about 90%, 95%, 96%, 97%, 98%, 99%, or 99.5% sequence identity to the sequence set forth in SEQ ID NO:24, or a portion thereof. In some embodiments, the HIS tag comprises the sequence set forth in SEQ ID NO:24. In some embodiments, the SUMO tag comprises a sequence that has at least at or about 90%, 95%, 96%, 97%, 98%, 99%, or 99.5% sequence identity to the sequence set forth in SEQ ID NO:41, or a portion thereof. In some embodiments, the SUMO tag comprises the sequence set forth in SEQ ID NO:41.

In some embodiments, the fusion protein comprises a sequence that has at least at or about 90%, 95%, 96%, 97%, 98%, 99%, or 99.5% sequence identity to the sequence set forth in SEQ ID NO:38. In some embodiments, the fusion protein comprises the sequence set forth in SEQ ID NO:38.

In some embodiments, the fusion protein comprises a sequence that has at least at or about 90%, 95%, 96%, 97%, 98%, 99%, or 99.5% sequence identity to the sequence set forth in SEQ ID NO:40. In some embodiments, the fusion protein comprises the sequence set forth in SEQ ID NO:40.

### E. Dimers

Provided herein are dimers of fusion proteins, comprising two or more monomers of any of the fusion proteins disclosed herein. In some aspects, provided herein is a dimer, comprising a first fusion protein molecule comprising any one of the fusion proteins disclosed herein, for example in Section II.E, and a second fusion protein molecule comprising any one of the fusion proteins disclosed herein, for example in Section II.E.

In some embodiments, the dimer is a homodimer. In some embodiments, the dimer is a heterodimer. In some embodiments, the dimers are formed by non-covalent interaction between the monomers. In some embodiments, the dimers are formed by covalent interaction between the monomers. In some aspects, the dimers comprise two or more Phi29 polymerase domains.

In some embodiments, the homodimer comprises a first Phi29 polymerase-kinesin 1 dimerization domain fusion protein and a second Phi29 polymerase-kinesin 1 dimerization domain fusion protein.

In some embodiments, the first fusion protein comprises from N-terminal to C-terminal a Phi29 polymerase domain, a linker, a kinesin 1 dimerization domain, and a tag. In some embodiments, the first fusion protein of the homodimer comprises from N-terminal to C-terminal SEQ ID NO:3, SEQ ID NO:9, SEQ ID NO: 18, and SEQ ID NO:43. In some embodiments, the second fusion protein of the homodimer comprises from N-terminal to C-terminal a Phi29 polymerase domain, a linker, a kinesin 1 dimerization domain, and a tag. In some embodiments, the second fusion protein of the homodimer provided herein comprises from N-terminal to C-terminal SEQ ID NO:3, SEQ ID NO:9, SEQ ID NO: 18, and SEQ ID NO:43. In some embodiments, the first fusion protein of the homodimer comprises a sequence that has at least at or about 90%, 95%, 96%, 97%, 98%, 99%, or 99.5% sequence identity to the sequence set forth in SEQ ID NO: 28 or 30. In some embodiments, the first fusion protein of the homodimer comprises a sequence that has at least at or about 90%, 95%, 96%, 97%, 98%, 99%, or 99.5% sequence identity to the sequence set forth in SEQ ID NO: 28. In some embodiments, the first fusion protein of the homodimer comprises a sequence that has at least at or about 90%, 95%, 96%, 97%, 98%, 99%, or 99.5% sequence identity to the sequence set forth in SEQ ID NO:30. In some embodiments, the first fusion protein comprises the sequence set forth in SEQ ID NO:28. In some embodiments, the first fusion protein comprises the sequence set forth in SEQ ID NO:30. In some embodiments, the second fusion protein of the homodimer comprises a sequence that has at least at or about 90%, 95%, 96%, 97%, 98%, 99%, or 99.5% sequence identity to the sequence set forth in SEQ ID NO: 28 or 30. In some embodiments, the second fusion protein of the homodimer comprises a sequence that has at least at or about 90%, 95%, 96%, 97%, 98%, 99%, or 99.5% sequence identity to the sequence set forth in SEQ ID NO: 28. In some embodiments, the second fusion protein of the homodimer comprises a sequence that has at least at or about 90%, 95%, 96%, 97%, 98%, 99%, or 99.5% sequence identity to the sequence set forth in SEQ ID NO:30. In some embodiments, the second fusion protein comprises the sequence set forth in SEQ ID NO:28. In some embodiments, the second fusion protein comprises the sequence set forth in SEQ ID NO:30.

In some embodiments, the first fusion protein comprises from N-terminal to C-terminal a variant Phi29 polymerase domain, a linker, a kinesin 1 dimerization domain, and a tag. In some embodiments, the first fusion protein of the homodimer comprises from N-terminal to C-terminal SEQ ID NO:7, SEQ ID NO:9, SEQ ID NO: 18, and SEQ ID NO:43. In some embodiments, the second fusion protein of the homodimer comprises from N-terminal to C-terminal a variant Phi29 polymerase domain, a linker, a kinesin 1 dimerization domain, and a tag. In some embodiments, the second fusion protein of the homodimer provided herein comprises from N-terminal to C-terminal SEQ ID NO:7, SEQ ID NO:9, SEQ ID NO:18, and SEQ ID NO:43. In some embodiments, the first fusion protein of the homodimer comprises a sequence that has at least at or about 90%, 95%, 96%, 97%, 98%, 99%, or 99.5% sequence identity to the sequence set forth in SEQ ID NO: 49. In some embodiments, the second fusion protein of the homodimer comprises a sequence that has at least at or about 90%, 95%, 96%, 97%, 98%, 99%, or 99.5% sequence identity to the sequence set forth in SEQ ID NO: 49. In some embodiments, the first fusion protein comprises the sequence set forth in SEQ ID NO:49. In some embodiments, the second fusion protein comprises the sequence set forth in SEQ ID NO:49.

In some embodiments, the heterodimer comprises a first Phi29 polymerase domain-SpyCatcher domain fusion protein and a second Phi29 polymerase domain-SpyTag domain fusion protein. In some embodiments, the first fusion protein comprises from N-terminal to C-terminal a Phi29 polymerase domain, a linker, SpyCatcher domain, and a tag. In some embodiments, the first fusion protein of the heterodimer comprises from N-terminal to C-terminal SEQ ID NO:3, SEQ ID NO:9, SEQ ID NO:22, and SEQ ID NO:43. In some embodiments, the second fusion protein of the heterodimer comprises from N-terminal to C-terminal a Phi29 polymerase domain, a linker, a SpyTag domain, and a tag. In some embodiments, the second fusion protein of the heterodimer comprises from N-terminal to C-terminal SEQ ID NO:3, SEQ ID NO:9, SEQ ID NO:24, and SEQ ID NO:43. In some embodiments, the first fusion protein of the heterodimer comprises a sequence that has at least at or about 90%, 95%, 96%, 97%, 98%, 99%, or 99.5% sequence identity to the sequence set forth in SEQ ID NO:34. In some embodiments, the first fusion protein comprises the sequence set forth in SEQ ID NO:34. In some embodiments, the second fusion protein of the heterodimer comprises a sequence that has at least at or about 90%, 95%, 96%, 97%, 98%, 99%, or 99.5% sequence identity to the sequence set forth in SEQ ID NO: 36. In some embodiments, the second fusion protein comprises the sequence set forth in SEQ ID NO:36.

In some embodiments, the Phi29 fusion protein comprises a sequence that has at least at or about 90%, 95%, 96%, 97%, 98%, 99%, or 99.5% sequence identity to the sequence set forth in SEQ ID NO: 1 or 3 fused to a sequence that has at least at or about 90%, 95%, 96%, 97%, 98%, 99%, or 99.5% sequence identity to the sequence set forth in SEQ ID NO: 18. In some embodiments, the Phi29 fusion protein comprises a sequence that has at least at or about 90%, 95%, 96%, 97%, 98%, 99%, or 99.5% sequence identity to the sequence set forth in SEQ ID NO: 1 or 3 fused to a sequence that has at least at or about 90%, 95%, 96%, 97%, 98%, 99%, or 99.5% sequence identity to the sequence set forth in SEQ ID NO:26. In some embodiments, the Phi29 fusion protein comprises a sequence that has at least at or about 90%, 95%, 96%, 97%, 98%, 99%, or 99.5% sequence identity to the sequence set forth in SEQ ID NO: 1 or 3 fused to a sequence that has at least at or about 90%, 95%, 96%, 97%, 98%, 99%, or 99.5% sequence identity to the sequence set forth in SEQ ID NO:22. In some embodiments, the Phi29 fusion protein comprises a sequence that has at least at or about 90%, 95%, 96%, 97%, 98%, 99%, or 99.5% sequence identity to the sequence set forth in SEQ ID NO: 1 or 3 fused to a sequence that has at least at or about 90%, 95%, 96%, 97%, 98%, 99%, or 99.5% sequence identity to the sequence set forth in SEQ ID NO:24. In some embodiments, the Phi29 fusion protein comprises a sequence that has at least at or about 90%, 95%, 96%, 97%, 98%, 99%, or 99.5% sequence identity to the sequence set forth in SEQ ID NO:51 fused to a sequence that has at least at or about 90%, 95%, 96%, 97%, 98%, 99%, or 99.5% sequence identity to the sequence set forth in SEQ ID NO:41.

### F. Exemplary Features of Fusion Proteins and Dimers

In some aspects, the provided fusion proteins exhibit one or more improved features or properties. These features or properties relate to the activity and function of a nucleic acid polymerase, such as, but not limited, DNA amplification. In some aspects, the fusion protein comprising a Phi29 polymerase domain and one or more polypeptide domains is provided in solution to perform amplification in a sample comprising a tissue or a cell.

In some embodiments, the Phi29 fusion protein exhibits one or more features selected from among improved amplification product homogeneity, improved solubility, improved nucleic acid binding affinity, improved processivity, improved fidelity, and/or improved polymerization rate, compared to a reference Phi29 polymerase. In some embodiments, the Phi29 fusion protein exhibits improved amplification product homogeneity compared to a reference Phi29 polymerase. In some embodiments, the Phi29 fusion protein generates a higher density of detected rolling-circle amplification (RCA) products in an RCA compared to a reference Phi29 polymerase. In some embodiments, the Phi29 fusion protein generates a higher signal intensity of RCA products in an RCA compared to a reference Phi29 polymerase. In some embodiments, the Phi29 fusion protein generates a smaller or more compact tightly compacted RCA product compared to a referenced Phi29 polymerase. In some embodiments, the Phi29 fusion protein generates a more homogenous RCA products compared to a referenced Phi29 polymerase. In some embodiments, the Phi29 fusion protein exhibits improved solubility compared to a reference Phi29 polymerase. In some embodiments, the Phi29 fusion protein exhibits improved nucleic acid binding affinity compared to a reference Phi29 polymerase. In some embodiments, the Phi29 fusion protein exhibits improved processivity compared to a reference Phi29 polymerase. In some embodiments, the Phi29 fusion protein exhibits improved fidelity compared to a reference Phi29 polymerase. In some embodiments, the Phi29 fusion protein exhibits improved polymerization rate compared to a reference Phi29 polymerase. In some embodiments, the reference Phi29 polymerase is a wild-type Phi29 polymerase, or a Phi29 polymerase that does not comprise the one or more protein-protein interaction domains or the one or more heterologous nucleic acid binding domains.

In some cases, an improved polymerization rate may be a slower polymerization rate compared to a reference polymerase (e.g., wild-type Phi29 polymerase). In some cases, an improved polymerization rate may be a faster polymerization rate compared to a reference polymerase (e.g., wild-type Phi29). Other exemplary features may relate to increased yield, altered cofactor selectivity, exonuclease deficiency, and increased resistance to photodamage. In some aspects, the provided Phi29 fusion proteins exhibit improved DNA binding affinity, stability, processivity, product uniformity, solubility, or yield, or that otherwise exhibit an improved ability to read through damaged, modified, or other difficult stretches of nucleic acid template, can be employed for precision amplification applications (e.g., RCA).

In some aspects described herein, the reference polymerase is a wild-type Phi29 polymerase, such as any wild-type Phi29 polymerase described herein. In some embodiments, the reference Phi29 polymerase is a wild-type Phi29 polymerase, or a Phi29 polymerase that does not comprise the one or more protein-protein interaction domains or the one or more heterologous nucleic acid binding domains. In some aspects described herein, the reference polymerase is a Phi29 polymerase that does not comprise the one or more amino acid substitutions, such as any wild-type Phi29 polymerase described herein. In some aspects, the reference polymerase is a Phi29 polymerase (e.g., a wild-type Phi29 polymerase), and the Phi29 fusion protein exhibits one or more improved features relative to the Phi29 polymerase. In some aspects described herein, the reference polymerase is a Phi29 polymerase that comprises one or more amino acid substitutions, such as any variant Phi29 polymerase described herein. In some aspects, the reference polymerase is a Phi29 polymerase (e.g., a variant Phi29 polymerase), and the Phi29 fusion protein exhibits one or more improved features relative to the Phi29 polymerase. In some embodiments, the provided Phi29 fusion proteins produce more amplified DNA at elevated temperatures compared to a reference Phi29 polymerase. In some embodiments, the increase in nucleic acid amplification and the generation of amplified nucleic acid product are measured or assessed using a fluorescent dye.

In some embodiments, the one or more features is of the fusion protein comprising a Phi29 polymerase domain is improved compared to a reference Phi29 polymerase (e.g., wild-type Phi29). In some embodiments, the one or more features is assayed under the same conditions using a comparable amount of the reference polymerase. Improvements in one or more features support an expanded application and/or utility of the Phi29 fusion protein, for example for DNA amplification and/or sequencing. In some embodiments, the provided Phi29 fusion protein possesses one or more features resulting in improved nucleic acid amplification from a biological sample and/or nucleic acid amplification methods such as a rolling circle amplification (RCA). In some cases, wild-type Phi29 has certain limitations, which can result in slower reaction kinetics, less amplification product, and greater heterogeneity and amplification bias, especially with DNA having high guanine/cytosine (G/C) content.

In some embodiments, the Phi29 fusion protein exhibiting one or more improved or altered features (e.g., product homogeneity, processivity, sensitivity, product density, and/or signal detection intensity) described herein is used for a nucleic acid amplification process, wherein the nucleic acid amplification is an RCA reaction. In some embodiments, the provided Phi29 fusion proteins, for example, those described in Section II.E., exhibit one or more altered or improved features, such as product uniformity, processivity, and/or detection signal intensity, improved amplification product homogeneity, higher RCA product density, higher RCA product intensity, smaller RCA product, or more homogenous RCA product, compared to a reference Phi29 polymerase.

In some aspects, polymerase processivity is the ability of a nucleic acid polymerase to carry out continuous nucleic acid synthesis on a template nucleic acid without frequent dissociation. Processivity can be measured by the average number of nucleotides incorporated by a nucleic acid polymerase on a single association-disassociation event. In any of the embodiments herein, the Phi29 fusion protein may exhibit an average processivity of at least 10 kb, at least 20 kb, at least 30 kb, at least 40 kb, at least 50 kb, at least 60 kb, or at least 70 kb. In some embodiments, a Phi29 fusion protein described herein exhibits improved processivity relative to a reference polymerase, wherein the improved processivity improves polymerase activity. In some embodiments, the Phi29 fusion protein exhibits improved processivity compared to a reference polymerase (e.g., a wild-type Phi29 polymerase). In some cases, a Phi29 fusion protein exhibits improved processivity compared to a reference polymerase (e.g., a wild-type Phi29 polymerase). In some embodiments, the provided Phi29 fusion protein produces more amplified nucleic acid compared to a reference polymerase. In some embodiments, the provided Phi29 fusion protein amplifies nucleic acid faster compared to a reference polymerase. In some embodiments, the nucleic acid amplification is an RCA reaction. In some embodiments, the provided Phi29 fusion protein generates a higher density of detected RCA products in an RCA reaction compared to a reference polymerase. In some embodiments, the provided Phi29 fusion protein generates a higher product signal intensity in an RCA reaction compared to a reference polymerase.

In some embodiments, the RCA reaction is assessed or evaluated for generated RCA product (RCP) signal intensity, from detection of the produced RCA product. In some aspects, the product signal intensity is calculated as a local signal intensity over a local background mean intensity. In some embodiments, the provided Phi29 fusion proteins generate a higher product signal intensity in an RCA reaction compared to a reference Phi29 polymerase. In some embodiments, the provided Phi29 fusion proteins produce an RCA product signal intensity at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins produce an RCA product signal intensity at least between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the RCA reaction is assessed or evaluated for generated RCA product (RCP) signal intensity, from detection of the produced RCA product. In some aspects, the product signal intensity is calculated as a local signal intensity over a local background mean intensity. In some embodiments, the provided Phi29 fusion proteins generate a higher product signal intensity in an RCA reaction compared to a reference Phi29 polymerase. In some embodiments, the provided Phi29 fusion proteins produce an RCA product signal intensity at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins produce an RCA product signal intensity at least between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the RCA reaction is assessed or evaluated for generated RCA product (RCP) signal-to-noise ratio (SNR), from detection of the produced RCP. In some aspects, the product signal-to-noise ratio (SNR) can be calculated as the difference between peak signal and background signal, divided by the square root of the background signal; or the difference between peak signal and background signal, divided by the Root Mean Square (RMS) value of the noise on the background signal. In some embodiments, the provided Phi29 fusion proteins generate a higher SNR of RCP detection compared to a reference Phi29 polymerase. In some embodiments, the provided Phi29 fusion proteins produce an SNR at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins produce an SNR at least between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the RCA reaction is assessed or evaluated for the distribution of different sizes of the generated RCA product (RCP), and the uniformity of the distribution is determined. In some aspects, the uniformity of the generated RCP can be a function of the standard deviation of the sizes of the generated RCPs, assuming a normal distribution of the sizes. In some embodiments, a Phi29 fusion protein described herein exhibits a product uniformity at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, a Phi29 fusion protein described herein exhibits a product uniformity at least between 10% and 200%, 25% and 175%, 50% and 150%, 75% and 125%, 90% and 110%, or 95% and 105% higher to a reference polymerase (e.g., a wild-type Phi29 polymerase). In some embodiments, a Phi29 fusion protein described herein exhibits a standard deviation of size distribution of the generated RCP at or about or at least at or about 1%, 5%, 10%, 15%, 20%, 25%, 30%, 40%, 50%, 75%, 100%, 150%, or 200% lower compared to a reference polymerase (e.g., a wild-type Phi29 polymerase). In some embodiments, a Phi29 fusion protein described herein exhibits a standard deviation of size distribution of the generated RCP at least between 10% and 200%, 25% and 175%, 50% and 150%, 75% and 125%, 90% and 110%, or 95% and 105% lower to a reference polymerase (e.g., a wild-type Phi29 polymerase). In some embodiments, RCA reactions performed using the provided Phi29 fusion protein generates RCPs with an average diameter less than 500 nm. In some embodiments, at least 50%, 55%, 60%, 65%, 70%, 75%, 80%, 85%, 90%, 95%, 96%, 97%, 98%, 99% or 99.5% or 100% of all RCPs generated by the provided Phi29 fusion protein has a diameter of less than 500 nm.

In some embodiments, a Phi29 fusion protein described herein produces an RCP size homogeneity at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, a Phi29 fusion protein described herein produces an RCP size homogeneity at least between 10% and 200%, 25% and 175%, 50% and 150%, 75% and 125%, 90% and 110%, or 95% and 105% higher to a reference polymerase (e.g., a wild-type Phi29 polymerase).

In some embodiments, the Phi29 fusion protein produces smaller or more compact RCA products at or about or at least at or about 1%, 5%, 10%, 15%, 20%, 25%, 30%, 40%, 50%, 75%, or 100%, smaller or more compact compared to a reference polymerase (e.g., a wild-type Phi29 polymerase). In some embodiments, a Phi29 fusion protein described herein produces smaller or more compact RCA products between 10% and 100%, 25% and 75%, 50% and 100%, 75% and 10%, or 00% and 50%, smaller compared to a reference polymerase (e.g., a wild-type Phi29 polymerase). In some embodiments, a Phi29 fusion protein described herein produces smaller or compact RCA products compared to a reference polymerase (e.g., a wild-type Phi29 polymerase) in similar amplification reaction conditions (e.g., same concentration of reagents and reaction time). In some embodiments, a Phi29 fusion protein described herein produces smaller or more compact RCA products due to slower activity compared to a reference polymerase (e.g., a wild-type Phi29 polymerase) in similar amplification reaction conditions (e.g., same concentration of reagents and reaction time).

In some embodiments, the provided Phi29 fusion proteins are used to amplify nucleic acids, e.g., DNA, using a particular method, such as in a rolling-circle amplification (RCA) reaction. In some embodiments, the provided Phi29 fusion proteins generate a higher density of detected rolling-circle amplification (RCA) products in an RCA reaction compared to a reference Phi29 polymerase. In some embodiments, the provided Phi29 fusion proteins generate a higher density of detected rolling-circle amplification (RCA) products in an RCA reaction compared to a reference Phi29 polymerase. In some embodiments, determining the density of detected RCA products (RCPs) involves the use and detection of a specific fluorescent dye, wherein the density of detected RCA products is measured in RCA product counts per μm² sample area. In some embodiments, the density of detected RCPs is related to the total sample signal. In some embodiments, the provided Phi29 polymerase generates a higher density of detected RCPs in an RCA reaction compared to a reference polymerase. In some embodiments, the provided Phi29 fusion proteins produce a density of detected RCA products that is at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins produce a density of detected RCA products that is between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the RCA reaction is assessed or evaluated for rolling circle amplification (RCA) assay sensitivity. In some aspects, sensitivity is calculated based on the detected object density per particular unit area. In some aspects, sensitivity is calculated based on the density of detected RCPs per nuclei area. In some embodiments, the provided Phi29 fusion proteins generate a higher sensitivity in an RCA assay compared to a reference Phi29 polymerase. In some embodiments, the provided Phi29 fusion proteins exhibit sensitivity in an RCA assay of at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins exhibit sensitivity in an RCA assay of at least between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the provided Phi29 fusion proteins generate a higher solubility in solution compared to a reference Phi29 polymerase. In some embodiments, the provided Phi29 fusion proteins exhibit solubility of at or about or at least at or about 1%, 5%, 10%, 15%, 20%, 25%, 30%, 40%, 50%, 75%, 100%, 150%, or 200% higher compared to a reference polymerase (e.g., a wild-type Phi29 polymerase). In some embodiments, the provided Phi29 fusion proteins exhibit solubility of at least between 10% and 200%, 25% and 175%, 50% and 150%, 75% and 125%, 90% and 110%, or 95% and 105% higher compared to a reference polymerase (e.g., a wild-type Phi29 polymerase).

In some embodiments, the provided Phi29 fusion proteins described herein further exhibit a longer half-life in the absence of substrate compared to a reference Phi29 polymerase. In some embodiments, the provided Phi29 fusion protein described herein further exhibits a longer half-life in the presence of substrate compared to a reference Phi29 polymerase. In some embodiments, the provided Phi29 fusion protein is more stable in the presence and absence of substrate compared to a reference Phi29 polymerase. In some embodiments, the Phi29 fusion protein substrate comprises a nucleotide, a nucleotide derivative, a polynucleotide, or a combination thereof. In some embodiments, the Phi29 fusion protein exhibits an extended half-life that is or about 5%, 10%, 15%, 20%, 25%, 30%, 40%, 50%, 75%, 100%, 150%, 200%, 300%, 400%, or 500% longer compared to a reference Phi29 polymerase in the presence or absence of substrate. In some embodiments, the provided Phi29 fusion protein described herein exhibits an extended half-life between 5% and 500%, 15% and 300%, 50% and 400%, 30% and 100%, 20% and 200%, 40% and 150%, 75% and 400%, or 50% and 300% longer compared to a reference Phi29 polymerase in the presence or absence of substrate.

In some embodiments, the Phi29 fusion protein described herein exhibits improved thermostability. In some aspects, the improved thermostability includes improved polymerase activity. In some embodiments, the improved thermostability includes improved, more rapid, more robust, and/or more accurate polymerase activity, for example at elevated temperatures. In some embodiments, the Phi29 fusion protein exhibiting improved thermostability can be used for a nucleic acid amplification reaction, such as a DNA amplification reaction.

Exemplary fluorescent dyes for measuring the increase in nucleic acid amplification and the generation of amplified nucleic acid product, such as in an RCA reaction, include any described herein. In some embodiments, the Phi29 fusion protein produces at or about or more than at or about 1%, 5%, 10%, 15%, 20%, 25%, 30%, 40%, 50%, 75%, 100%, 150%, or 200% more amplified DNA compared to a reference polymerase. In some embodiments, the Phi29 fusion protein produces between 10% and 200%, 25% and 175%, 50% and 150%, 75% and 125%, 90% and 110%, or 95% and 105% more amplified DNA compared to a reference polymerase.

### III. Nucleic Acids, Vectors and Expression Systems

Also provided herein is a polynucleotide, vector, or expression system encoding any of the provided Phi29 fusion proteins, for example, those described in Section II.E.

The nucleic acids may include those encompassing natural and/or non-naturally occurring nucleotides and bases, e.g., including those with backbone modifications. The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some embodiments, a Phi29 fusion protein may be a recombinantly produced. In some aspects, the nucleic acid is synthetic. In some aspects, the nucleic acid molecule can be modified (e.g., codon optimization) for use in the constructs described herein. In some embodiments, the sequences can be designed to contain terminal restriction site sequences for purposes of cloning into vectors.

### A. Polynucleotides

In some aspects, provided herein is a polynucleotide sequence encoding any of the provided Phi29 fusion proteins, such as any of those described in Section II.E. In some aspects, the encoded Phi29 fusion proteins exhibit or possess one or more of the exemplary features or properties, such as any of those described in Section II.G.

In some embodiments, provided herein are polynucleotides encoding fusion protein comprising a Phi29 polymerase domain. In some embodiments, the polynucleotides encode any of the provided fusion Phi29 polymerases described herein, such as those described in Section II.A.

In some embodiments, provided polynucleotides include polynucleotides encoding a wild-type Phi29 polymerase domain, wherein the polynucleotide comprises the sequence set forth in SEQ ID NO:2. In some embodiments, the polynucleotide comprises a sequence that has at least at or about 90%, 95%, 96%, 97%, 98%, 99%, or 99.5% sequence identity to the sequence set forth in SEQ ID NO:2. In some embodiments, provided polynucleotides include polynucleotides encoding a wild-type Phi29 polymerase, wherein the polynucleotide comprises the sequence set forth in SEQ ID NO:4. In some embodiments, the polynucleotide comprises a sequence that has at least at or about 90%, 95%, 96%, 97%, 98%, 99%, or 99.5% sequence identity to the sequence set forth in SEQ ID NO:4.

In some embodiments, provided polynucleotides include polynucleotides encoding a protein-protein interaction domain, such as a dimerization domain. In some embodiments, provided polynucleotides include polynucleotides encoding a kinesin 1 dimerization domain. In some embodiments, the polynucleotides encode any of the provided kinesin 1 dimerization domains described herein, such as those described in Section II.B. In some embodiments, provided polynucleotides include polynucleotides encoding a kinesin 1 dimerization domain, wherein the polynucleotide comprises a sequence that has at least at or about 90%, 95%, 96%, 97%, 98%, 99%, or 99.5% sequence identity to the sequence set forth in SEQ ID NO: 19, or a portion thereof. In some embodiments, provided polynucleotides include polynucleotides encoding a kinesin 1 dimerization domain, wherein the polynucleotide comprises the sequence set forth in SEQ ID NO: 19.

In some embodiments, provided polynucleotides include polynucleotides encoding a SpyCatcher domain. In some embodiments, the polynucleotides encode any of the provided SpyCatcher domains described herein, such as those described in Section II.B. In some embodiments, provided polynucleotides include polynucleotides encoding a SpyCatcher domain, wherein the polynucleotide comprises the sequence set forth in SEQ ID NO:23, or a portion thereof. In some embodiments, the polynucleotide comprises a sequence that has at least at or about 90%, 95%, 96%, 97%, 98%, 99%, or 99.5% sequence identity to the sequence set forth in SEQ ID NO:23.

In some embodiments, provided polynucleotides include polynucleotides encoding a SpyTag domain. In some embodiments, the polynucleotides encode any of the provided SpyTag domains described herein, such as those described in Section II.B. In some embodiments, provided polynucleotides include polynucleotides encoding a SpyTag domain, wherein the polynucleotide comprises the sequence set forth in SEQ ID NO:25, or a portion thereof. In some embodiments, the polynucleotide comprises a sequence that has at least at or about 90%, 95%, 96%, 97%, 98%, 99%, or 99.5% sequence identity to the sequence set forth in SEQ ID NO:25.

In some embodiments, provided polynucleotides include polynucleotides encoding a nucleic acid binding domain. In some embodiments, provided polynucleotides include polynucleotides encoding a HU domain. In some embodiments, the polynucleotides encode any of the provided HU domains described herein, such as those described in Section II.C. In some embodiments, provided polynucleotides include polynucleotides encoding a HU domain, wherein the polynucleotide comprises the sequence set forth in SEQ ID NO:27, or a portion thereof. In some embodiments, the polynucleotide comprises a sequence that has at least at or about 90%, 95%, 96%, 97%, 98%, 99%, or 99.5% sequence identity to the sequence set forth in SEQ ID NO:27.

In some embodiments, provided polynucleotides include polynucleotides encoding a variant Phi29 polymerase. In some embodiments, the polynucleotides encode variant Phi29 polymerases comprising one or more amino acid modifications or variations (e.g., substitutions, insertions, deletions, and/or combinations thereof) compared to the sequence of the wild-type Phi29 polymerase. Amino acid sequence variants of a Phi29 polymerase may be prepared by introducing appropriate modifications into the nucleotide sequence encoding the wild-type Phi29 polymerase. Such modifications include, for example, deletions from and/or insertions into and/or substitutions of residues within the nucleotide sequences encoding the wild-type Phi29 polymerase. The encoded variant Phi29 polymerase can comprise any combination of deletion, insertion, and substitutions. In some aspects, the encoded variant Phi29 polymerase exhibits desired characteristics, e.g., improved polymerase activity, features and/or properties. In some embodiments, the polynucleotides encode any of the provided variant Phi29 polymerases described herein, such as those described in Section II.A. In some embodiments, provided polynucleotides include polynucleotides encoding a variant Phi29, wherein the polynucleotide comprises the sequence set forth in SEQ ID NO:8, or a portion thereof. In some embodiments, the polynucleotide comprises a sequence that has at least at or about 90%, 95%, 96%, 97%, 98%, 99%, or 99.5% sequence identity to the sequence set forth in SEQ ID NO:8.

In some embodiments, the polynucleotide sequences encoding the provided Phi29 fusion protein or a portion thereof is codon-optimized for a host cell or a recombinant expression system to generate the Phi29 fusion protein. Typically, codon optimization involves balancing the percentages of codons selected with the published abundance of transfer RNAs appropriate for the host cell or expression system so that none is overloaded or limiting. This may be necessary in some cases because most amino acids are encoded by more than one codon, and codon usage varies from organism to organism. Differences in codon usage between transfected genes and host cells can have effects on protein expression and immunogenicity of a nucleic acid construct. In general, for codon optimization, codons are chosen to select for those codons that are in balance with the host cell usage frequency. Typically, the redundancy of the codons for amino acids is such that different codons code for one amino acid. In some embodiments, in selecting a codon for replacement, it may be desired that the resulting mutation is a silent mutation such that the codon change does not affect the amino acid sequence. Generally, the last nucleotide of the codon can remain unchanged without affecting the amino acid sequence.

### B. Recombinant Nucleic Acids and Vectors

In some aspects, provided herein are recombinant nucleic acids comprising polynucleotides encoding any of the Phi29 fusion proteins, such as those described in Section III.A. In some aspects, provided herein are vectors comprising polynucleotides encoding any of the Phi29 fusion proteins, such as those described in Section II. In some embodiments, the recombinant nucleic acid or vector comprises a polynucleotide encoding a Phi29 fusion protein described herein, that comprises one or more amino acid modifications or variations, e.g., substitutions, deletions, insertions, and/or mutations, compared to the sequence of the wild-type Phi29 polymerase.

In some embodiments, the recombinant nucleic acid molecule comprises a polynucleotide described in Section III.A.

In some embodiments, the recombinant nucleic acid molecule further comprises a transcription regulatory sequence operatively linked with the polynucleotide. In some embodiments, the transcription regulatory sequence comprises a promoter selected from among a bacterial, viral, and mammalian promoter. In some embodiments, the promoter is a non-viral promoter or a viral promoter, such as a cytomegalovirus (CMV) promoter, an SV40 promoter, an RSV promoter, and a promoter found in the long-terminal repeat of the murine stem cell virus. Other suitable promoters can also be used.

In some embodiments, a vector is provided comprising a polynucleotide described in Section III.A or a recombinant nucleic acid molecule described herein, such as those encoding any of the Phi29 fusion proteins described herein. In some embodiments, the vector is a plasmid, a viral vector, a cosmid, or a transposon. The expression vector can be any suitable recombinant expression vector, and can be used to transform or transfect any suitable host. Suitable vectors include those designed for propagation and expansion or for expression or both, such as plasmids and viruses. In some embodiments, the vector is an expression vector. Exemplary vectors can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as AG10, AGT11, AZapII (Stratagene), AEMBL4, and ANM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some cases, the vector is a viral vector. In some such aspects, the viral vector is a retroviral vector, such as a lentiviral vector.

In some aspects, provided herein is a recombinant nucleic acid molecule comprising any one of the polynucleotides disclosed herein. In some embodiments, the recombinant nucleic acid molecule further comprises a transcription regulatory sequence operatively linked with the polynucleotide. In some embodiments, the transcription regulatory sequence comprises a promoter selected from among a bacterial, viral, and mammalian promoter.

In some aspects, provided herein is a vector comprising any of the polynucleotides disclosed herein. In some aspects, provided herein is a vector comprising any one of the recombinant nucleic acid molecules disclosed herein. In some embodiments, the vector comprises a plasmid, a viral vector, a cosmid, or a transposon.

In some aspects, provided herein is a recombinant expression system comprising any one of the polynucleotides disclosed herein. In some aspects, provided herein is a recombinant expression system comprising any one of the recombinant nucleic acid molecules disclosed herein. In some aspects, provided herein is a recombinant expression system comprising any one of the vectors disclosed herein. In some embodiments, the polynucleotide, the recombinant nucleic acid molecule, or the vector is for use in transforming the recombinant expression system to generate a transformed recombinant expression system.

In some embodiments, the transformed recombinant expression system generates the fusion protein. In some embodiments, the recombinant expression system is a cell system or a cell-free system. In some embodiments, the recombinant expression system comprises one or more cell systems selected from among a bacterial cell, a fungal cell, an insect cell, and a mammalian cell. In some embodiments, the polynucleotide sequence encoding the fusion protein is codon-optimized for the recombinant expression system.

### C. Host Cells and Expression Systems

In some instances, provided herein are host cells or recombinant expression systems comprising any of the polynucleotides, recombinant nucleic acid molecules, or vectors provided herein, such as any that are described in Section III.B. In some embodiments, the polynucleotide, the recombinant nucleic acid molecule, and/or the vector is for use in transforming the host cell or recombinant expression system to generate a transformed host cell or recombinant expression system.

The polynucleotide, the recombinant nucleic acid molecule, and/or the vector can be introduced into the host cell or recombinant expression system using various methods for introduction or transfer of nucleic acids, including via viral, e.g., retroviral or lentiviral, transduction, transposons, and electroporation. In some aspects, the recombinant expression system comprises a host cell, such as any host cells described herein.

In some embodiments, the transformed host cell or recombinant expression system generates or encodes any of the Phi29 fusion proteins described herein, for example, a Phi29 fusion protein comprising an amino acid sequence having at least or about 90%, 95%, 96%, 97%, 98%, 99%, or 99.5% sequence identity to the sequence set forth in SEQ ID NO:28 or SEQ ID NO:38.

In some embodiments, the host cell or recombinant expression system is used to produce the Phi29 fusion protein. In some embodiments, the host cell or recombinant expression system is a cell system or a cell-free system. In some embodiments, the host cell or recombinant expression system comprises one or more cell systems selected from among a bacterial cell, a fungal cell, an insect cell, and a mammalian cell. In some embodiments, a cell system may be selected from a group comprising E. coli, S. cerevisiae, D. melanogaster, and CHO cells. For example, E. coli is a bacterial cell that can be transformed with the polynucleotide, recombinant nucleic acid, and/or vector described herein for effective production of a Phi29 fusion protein. In some aspects, in a bacterial host cell or expression system, such as E. coli, protein translation is initiated with N-formyl-methionine (fMet), which during translation initiation is brought to the ribosome in the form of fMet-tRNAfMet. In some aspects, the formylated methionine at the N-terminus of nascent bacterial proteins is typically removed co-translationally shortly after the nascent protein has emerged from the peptide exit tunnel of the ribosome, by peptide deformylase (PDF), and in the majority of cases, is followed by methionine excision by methionine aminopeptidase (MAP). Accordingly, in some aspects, bacterially produced proteins do not include an initial Met residue.

In some embodiments, the polynucleotide sequence encoding the Phi29 fusion protein is codon-optimized for the host cell or recombinant expression system, for example, as described in Section III.C.

### IV. Methods of Using and Uses of Recombinant Phi29 Fusion Proteins, and Compositions Thereof, and Applications

Also provided herein are methods that involve or employ any of the provided Phi29 fusion proteins, compositions or kits that include a Phi29 fusion protein, or nucleic acids or vectors that encode the Phi29 fusion proteins, and uses of any of the foregoing, such as for nucleic acid amplification and/or sequencing. In some aspects, the provided Phi29 fusion proteins are used in a variety analysis or assessment methods or experiments (e.g., sequencing, genotyping, and amplification reactions), for assessing a biological sample. In some aspects, the provided Phi29 fusion proteins are used in amplifying nucleic acids, e.g., DNAs or RNAs, including in applications such as rolling circle amplification (RCA). In some aspects, the provided Phi29 fusion proteins are provided in solution and contacted with a biological sample.

In some aspects, provided are methods for amplifying nucleic acids, such as DNA, that involve contacting a biological sample containing DNA to be amplified with any of the provided Phi29 fusion proteins and uses thereof, compositions or kits that include a Phi29 fusion proteins, or nucleic acids or vectors that encode the Phi29 fusion proteins.

In some aspects, provided are methods of performing a rolling circle amplification (RCA) that involves the use of any of the provided Phi29 fusion proteins, compositions or kits that include a Phi29 fusion protein, or nucleic acids or vectors that encode the Phi29 fusion proteins. In some aspects, the method of performing RCA involves contacting a biological sample containing nucleic acids, e.g., DNA, to be amplified with any of the provided Phi29 fusion proteins, compositions or kits that include a Phi29 fusion protein, or nucleic acids or vectors that encode the Phi29 fusion proteins. In some aspects, the methods thereby amplify the nucleic acid by RCA.

In some embodiments, the provided Phi29 fusion proteins are used in methods that involve the amplification of a nucleic acid probe through rolling circle amplification. In some embodiments, RCA involves the use of primary probes that recognize particular target nucleic acid sequences, and can be amplified via RCA to be detected. In some embodiments, the primary probes, such as a circularizable probe or a probe set that comprises a circularizable probe, contain one or more barcodes. In some embodiments, the barcodes are bound by detection primary probes, which do not need to be fluorescent, but that include a target-binding portion (e.g., for hybridizing to one or more primary probes) and multiple other barcodes (e.g., secondary barcodes, versus the primary barcodes on the primary probes). In some embodiments, the barcodes of the detection primary probes are targeted by detectably labelled detection oligonucleotides, such as fluorescently labelled oligonucleotides. In some embodiments, one or more decoding schemes are used to decode the signals, such as fluorescence, for sequence determination. Examples of decoding schemes are described in Eng et al., Nature 2019; 568(7751):235-239; Chen et al., Science; 2015; 348(6233): aaa6090; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, the provided Phi29 fusion proteins are used in methods that involve using a circular or circularizable construct hybridized to the nucleic acid of interest to generate a circular nucleic acid. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In some embodiments, the circular nucleic acid is a construct formed using ligation. In some embodiments, the circular construct is formed using template primer extension followed by ligation. In some embodiments, the circular construct is formed by providing an insert between ends to be ligated. In some embodiments, the circular construct is formed using a combination of any of the foregoing. In some embodiments, the ligation is a DNA-DNA templated ligation. In some embodiments, the ligation is an RNA-RNA templated ligation. Exemplary RNA-templated ligation probes and methods are described in US 2020/0224244 which is incorporated herein by reference in its entirety. In some embodiments, the ligation is an RNA-DNA templated ligation. In some embodiments, a splint is provided as a template for ligation.

Following formation of the circular nucleic acid, in some instances, an amplification primer is added. In some instances, the amplification primer is added with the primary and/or secondary probes. In some instances, the amplification primer may also be complementary to the target nucleic acid and the circularizable probe (e.g., a SNAIL probe). In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some embodiments, amplification of the circular nucleic acid is primed by the target nucleic acid (e.g., a target RNA). In some embodiments, the target nucleic acid is immobilized in the biological sample. In some embodiments, a target RNA is cleaved by an enzyme (e.g., RNase H). In some embodiments, the target nucleic acid is cleaved at a position downstream of the target sequences bound to the circular probe. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circular probe generated from a circularizable probe or probe set (e.g., target-primed RCA). In some cases, a plurality of nucleic acid oligonucleotides can be used to perform target-primed RCA for a plurality of different target nucleic acids.

In any of the embodiments herein, the biological sample is contacted with the RNase H (and optionally with the nucleic acid oligonucleotide) before or during formation of the circular probe. In some embodiments, the biological sample is contacted with the oligonucleotide and with the RNase H simultaneously or sequentially (in either order) before contacting the sample with the probe or probe set. In any of the embodiments herein, the RNase H comprises an RNase H1 and/or an RNAse H2. In some embodiments, RNase inactivating agents or inhibitors are added to the sample after cleaving the target RNA.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of a circularizable probe or probe set to generate a circularized probe) the circular or circularized probe is rolling circle amplified to generate an RCA product (RCP) containing multiple copies of the RCP.

Following amplification, the sequence of the amplicon (e.g., RCA product) or a portion thereof, is determined or otherwise analyzed, for example by using detectably labelled probes and imaging. The sequencing or analysis of the amplification products can comprise sequencing by hybridization, sequencing by ligation, and/or fluorescent in situ sequencing, and/or wherein the in situ hybridization comprises sequential fluorescent in situ hybridization. In some instances, a sequence of the RCA product is detected using, e.g., the secondary and higher order probes and detection oligonucleotides described herein.

In some instances, the provided Phi29 fusion proteins are used in methods for amplifying a DNA, such as a sequencing method, a genotyping method, or an amplification method. In some embodiments, the methods involve contacting a biological sample containing DNA to be amplified with any of the provided Phi29 fusion proteins, any compositions or kits that include a Phi29 fusion protein, or nucleic acids or vectors that encode the fusion proteins. In some embodiments, the method also involves incubating the biological sample and the Phi29 fusion proteins, compositions or kits that include a Phi29 fusion protein, or nucleic acids or vectors that encode the Phi29 fusion proteins.

In some embodiments, the biological sample containing DNA to be amplified with the Phi29 fusion protein is an isolated or synthetic nucleic acids. In some embodiments, the biological sample is tested in vitro. In some embodiments, the biological sample is an organ or a tissue sample. In some embodiments, the biological sample is tested in situ, for example, wherein the Phi29 fusion protein is added to a freshly or previously prepared tissue sample for target gene analysis within a tissue slice. In some embodiments, the DNA is amplified by a rolling circle amplification (RCA) as described in this section. In some embodiments, the DNA is amplified by in situ RCA in the biological sample.

In some instances, the provided Phi29 fusion proteins are used in methods of performing a rolling circle amplification (RCA). In some embodiments, the methods involve contacting a biological sample containing DNA to be amplified with the Phi29 fusion protein, any compositions or kits that include a Phi29 fusion protein, or nucleic acids or vectors that encode the Phi29 fusion proteins. In some embodiments, the methods also involve incubating the biological sample and the Phi29 fusion protein, any compositions or kits that include a Phi29 fusion protein, or nucleic acids or vectors that encode the Phi29 fusion proteins. In some embodiments, the Phi29 fusion protein used in the method of RCA can be any of those described in Section II.

In some embodiments, the method of RCA comprises incubating the biological sample and the Phi29 fusion protein at a specific temperature. In some embodiments, the temperature is optimal for maximizing activity of the Phi29 fusion protein. In some embodiments, the Phi29 fusion protein and the biological sample are incubated at a temperature of at or about 5° C., 10° C., 15° C., 20° C., 25° C., 30° C., 35° C., 40° ° C., 45° C., or higher. In some embodiments, the Phi29 fusion protein and the biological sample are incubated at a temperature between 5° C. and 50° C., 10° C. and 45° C., 25° C. and 45° C., 30° C. and 45° C., or 35° C. and 45° C. In some embodiments, the Phi29 fusion protein and the biological sample are incubated at an elevated temperature. In some embodiments, the Phi29 fusion protein and the biological sample are incubated at or about 31° C., 32° ° C., 33° C., 34° C., 35° C., 36° C., 37° C., 38° C., 39ºC, 40° C., 41ºC, 42° C., 43° C., 44° C., 45° C., 46° C., 47° ° C., 48° C., 49° C., or 50° C. In some embodiments, the Phi29 fusion protein and the biological sample are incubated between 30° ° C. and 50° C., 32° C. and 48° C., 34° C. and 46° ° C., 36° C. and 44° C., or 38° C. and 42° C.

In some embodiments, RCA is performed during incubation of the Phi29 fusion protein and the biological sample. In some embodiments, the RCA is performed for about 0.25, 0.5, 0.75, 1, 1.5, 2, 2.5, 3, 4, 5, 6, 8, 10, 12, 14, 16, 18, 20, 22, or 24 hours. In some embodiments, the RCA is performed for 0.25, 0.5, 0.75, 1, 1.5, 2, 2.5, 3, 4, 5, 6, 8, 10, 12, 14, 16, 18, 20, 22, or 24 hours. In some embodiments, the RCA is performed for at or about 15 minutes, 30 minutes, 45 minutes, 1 hour, 2 hours, 3 hours, 6 hours, 12 hours, or longer. In some embodiments, the RCA is performed for more than 24 hours. In some embodiments, the RCA is performed for between 1 hour and 8 hours, 0.25 hour and 12 hours, 4 hour and 24 hours, 2 hour and 16 hours, 3 hour and 10 hours, 4 hour and 18 hours, 5 hour and 14 hours, or 0.5 hour and 20 hours. In some embodiments, RCA is performed for no more than 12 hours. In some embodiments, RCA is performed for no more than 8 hours. In some embodiments, RCA is performed for no more than 6 hours. In some embodiments, the incubation time used is sufficient to generate a detectable amount of an RCA product.

In some embodiments, the method of performing RCA further comprises detecting the amplification product. In some embodiments, the amplification product is an RCA product (RCP). In some embodiments, the amplification product is detected by contacting the biological sample with a fluorescent dye or affinity molecule. In some embodiments, the biological sample is contacted with a fluorescent dye or affinity molecule before initiating the RCA reaction. In some embodiments, the amplification product is detected by contacting the biological sample with SYBR gold or SYBR green. SYBR dyes are asymmetrical cyanine dyes used for staining double-stranded DNA, single-stranded DNA, and RNA molecules. In some embodiments, the amplification product is detected by contacting the biological sample with ATTO-532 (Rhodamine 6G). In some embodiments, the amplification product is detected in real time during the amplification.

In some embodiments, the provided methods employ a Phi29 fusion protein that exhibits one or more of any of the features described herein, such as those described in Section II.E.

In some aspects, provided herein is a method for amplifying a nucleic acid, the method comprising contacting a biological sample containing a target nucleic acid to be amplified with any one of the Phi29 fusion proteins or dimers disclosed herein. In some embodiments, the nucleic acid is amplified by a rolling circle amplification (RCA). In some aspects, provided herein is a method of performing a rolling circle amplification (RCA), the method comprising contacting a biological sample containing a target nucleic acid to be amplified with any one of the Phi29 fusion proteins or dimers disclosed herein, thereby amplifying the nucleic acid by RCA. In some embodiments, the method further comprises contacting the biological sample with a circularizable probe. In some embodiments, the circularizable probe comprises one or more hybridization regions and one or more barcode regions. In some embodiments, the one or more barcode sequences correspond to a sequence of the target nucleic acid. In some embodiments, the one or more hybridization regions binds to and/or is complementary to the target nucleic acid or a portion of the target nucleic acid. In some embodiments, the method further comprises ligating the ends of the circularizable probe, optionally wherein the ligation is templated by the binding of the hybridization regions to the target nucleic acid or portion thereof, thereby generating a circularized probe. In some embodiments, the circularized probe is amplified by rolling circle amplification, to generate a rolling circle amplification product. In some embodiments, the rolling circle amplification product comprises multiple copies of a sequence complementary to the circularized probe.

In some embodiments, the method further comprises detecting the rolling circle amplification product. In some embodiments, the detecting comprises detecting a sequence in rolling circle amplification product, sequencing all or a portion of a sequence in rolling circle amplification product and/or in situ hybridization to a sequence in rolling circle amplification product. In some embodiments, the detecting comprises sequencing the complement of the one or more barcode sequences. In some embodiments, the detecting comprises hybridizing to the rolling circle amplification product a detection oligonucleotide labelled with a fluorophore, an isotope, a mass tag, or a combination thereof. In some embodiments, the detecting comprises imaging the rolling circle amplification product.

In some embodiments, the amplification is performed in situ in the biological sample. In some embodiments, the amplification is performed in a cell or tissue sample. In some embodiments, the detecting is performed in situ in the biological sample. In some embodiments, the detecting is performed in a cell or tissue sample. In some embodiments, the biological sample is a fixed and/or permeabilized biological sample. In some embodiments, the biological sample is a cell or tissue sample. In some embodiments, the target nucleic acid comprises deoxyribonucleotide residues. In some embodiments, the target nucleic acid comprises one or more ribonucleotide residues.

In some embodiments, the Phi29 fusion protein and the biological sample are contacted at a temperature of at or about 5° C., 10° C., 15° C., 20° C., 25° C., 30° C., 35° C., 37° C., 40° C., 42° ° C., 45° C., 50° C. or higher. In some embodiments, the RCA is performed for at or about 15 minutes, 30 minutes, 45 minutes, 1 hour, 2 hours, 3 hours, 6 hours, 12 hours, or longer.

In some embodiments, the Phi29 fusion protein or the dimer exhibits one or more features selected from among improved amplification product homogeneity, improved solubility, improved nucleic acid binding affinity, improved processivity, improved fidelity, and improved polymerization rate, compared to a reference Phi29 polymerase. In some embodiments, the Phi29 fusion protein or the dimer exhibits improved amplification product homogeneity compared to a reference Phi29 polymerase. In some embodiments, the Phi29 fusion protein or the dimer generates a higher density of detected rolling-circle amplification (RCA) products in an RCA compared to a reference Phi29 polymerase. In some embodiments, the Phi29 fusion protein or the dimer generates a higher signal intensity of RCA products in an RCA compared to a reference Phi29 polymerase. In some embodiments, the Phi29 fusion protein or the dimer generates a smaller or more compact (e.g., tightly compacted) RCA product compared to a referenced Phi29 polymerase. In some embodiments, the Phi29 fusion protein or the dimer generates a more homogenous RCA products compared to a referenced Phi29 polymerase. In some embodiments, the Phi29 fusion protein or the dimer exhibits improved solubility compared to a reference Phi29 polymerase. In some embodiments, the Phi29 fusion protein or the dimer exhibits improved nucleic acid binding affinity compared to a reference Phi29 polymerase. In some embodiments, the Phi29 fusion protein or the dimer exhibits improved processivity compared to a reference Phi29 polymerase. In some embodiments, the Phi29 fusion protein or the dimer exhibits improved fidelity compared to a reference Phi29 polymerase. In some embodiments, the Phi29 fusion protein or the dimer exhibits improved polymerization rate compared to a reference Phi29 polymerase. In some embodiments, the RCA is performed at a temperature of at or about 5° C., 10° C., 15° C., 20° C., 25° C., 30° ° C., 35° C., 37° C., 40° C., 42° ° C., 45° C., 50° C. or higher. In some embodiments, the reference Phi29 polymerase is a wild-type Phi29 polymerase, a Phi29 polymerase that does not comprise the one or more protein-protein interaction domains or the one or more heterologous nucleic acid binding domains, or a wild-type Phi29 polymerase monomer.

### A. Samples

A sample, such as a biological sample, that can be assessed or analyzed using any of the Phi29 fusion proteins, compositions or kits that include a Phi29 fusion proteins, or nucleic acids or vectors that encode the Phi29 fusion proteins as provided herein, can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaeon, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprises a cell pellet or a section of a cell pellet. In some embodiments, the biological sample is or comprises a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid, and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20TM), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### B. Analytes

The methods and compositions provided herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte) and may lead directly to the generation of an RCA template using a Phi29 fusion proteins provided herein (e.g., a circularizable probe or circular probe). Alternatively, the specific binding partner may be coupled to a nucleic acid, which may be detected using an RCA strategy, e.g., in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as DNA or RNA, e.g., interactions between proteins and nucleic acids, e.g., regulatory factors, such as transcription factors, and DNA or RNA.

### 1. Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### 2. Labeling Agents

In some embodiments, the methods and compositions provided herein can be used for analyzing endogenous analytes (e.g., RNA, ssDNA, cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labelling agent. For example, the reporter oligonucleotide may comprise a barcode sequence that permits identification of the labeling agent. In some cases, the sample contacted by the labelling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method involves one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### 3. Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., an amplification product or an extension product, for example, by a nucleic acid polymerase), a replication product, a transcription/reverse transcription product, and/or an amplification product such as a rolling circle amplification (RCA product) thereof is analyzed. In some embodiments, a labeling agent that directly or indirectly binds to an analyte in the biological sample is analyzed. In some embodiments, a product (e.g., an amplification product such as a rolling circle amplification (RCA product), an extension product (e.g., by a nucleic acid polymerase), a hybridization product, a ligation product, a replication product, a transcription/reverse transcription product), and/or of a labeling agent that directly or indirectly binds to an analyte in the biological sample is analyzed.

### C. Detection and Analysis

In some aspects, provided herein are methods comprising in situ assays, such as an in situ assay that involves the use of any of the Phi29 fusion proteins provided herein. In some aspects, the in situ assays involve generation of the amplification product in situ, for example using any of the Phi29 fusion proteins or compositions provided herein, and detection of the amplified product in situ, for example, using microscopy or other detection or determination methods involving an optical readout. In other aspects, the nucleic acid amplification and/or detection is performed in vitro.

In some aspects, detection or determination of a sequence of a target nucleic acid or amplification product thereof is performed in situ in a cell in an intact tissue. In some aspects, the methods involve in situ generation of nucleic acid amplification product, such as a rolling circle amplification (RCA) product using Phi29 fusion proteins provided herein, and detecting the amplification products.

In some embodiments, the method involves imaging the sample to detect a signal associated with a probe (e.g., circular or circularizable probe) hybridized to a target nucleic acid in the sample. In some embodiments, the probe hybridized to the target nucleic acid comprises one or more barcode sequences for detection and/or a means for signal amplification (e.g., RCA). In some embodiments, the signal can be amplified in situ in the sample. In some embodiments, the signal associated with the probe is amplified in the sample (e.g., using any of the Phi29 fusion proteins or compositions provided herein).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Some examples of techniques for in situ sequencing or in in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, the methods involve imaging the biological sample to detect a particular nucleic acid or an amplification product thereof. In some embodiments, a sequence of the rolling circle amplification product, or other generated product can be analyzed in situ in the biological sample. In some of any embodiments, the imaging can comprise detecting a signal associated with a fluorescently labelled probe that directly or indirectly binds to a rolling circle amplification product of the circularized probe. In some embodiments, the sequence of the rolling circle amplification product, extension product, or other generated product can be analyzed by sequential hybridization, sequencing by hybridization, sequencing by ligation, sequencing by synthesis, sequencing by binding, or a combination thereof. In some embodiments, barcodes or complements thereof (e.g., barcode sequences or complements thereof comprised by the probes disclosed herein or products thereof) can be analyzed (e.g., detected or sequenced) using any suitable method or technique, including those described herein, such as sequencing by synthesis (SBS), sequencing by ligation (SBL), or sequencing by hybridization (SBH). In some instances, barcoding schemes and/or barcode detection schemes as described in RNA sequential probing of targets (RNA SPOTs), single-molecule fluorescent in situ hybridization (smFISH), multiplexed error-robust fluorescence in situ hybridization (MERFISH) or sequential fluorescence in situ hybridization (seqFISH+) can be used. In any of the preceding implementations, the methods provided herein can include analyzing the barcodes by sequential hybridization and detection with a plurality of labelled probes (e.g., detection probes (e.g., detection oligos) or barcode probes). In some instances, the barcode detection steps can be performed as described in hybridization-based in situ sequencing (HybISS). In some instances, probes can be detected and analyzed (e.g., detected or sequenced) as performed in fluorescent in situ sequencing (FISSEQ), or as performed in the detection steps of the spatially-resolved transcript amplicon readout mapping (STARmap) method. In some instances, signals associated with an analyte can be detected as performed in sequential fluorescent in situ hybridization (seqFISH).

In some aspects, an in situ hybridization based assay is used to localize and analyze nucleic acid sequences (e.g., a probe comprising one or more specific barcode sequences of interest) within a native biological sample, e.g., a portion or section of tissue. In some embodiments, the in situ assay is used to analyze the presence, absence, an amount or level of the probe hybridized to a nuclei acid substrate in a biological sample, while preserving spatial context. In some aspects, the methods include detecting and processing signals from locations of amplification products generated using the provided polymerase fusion proteins and detected in sequential cycles.

In some embodiments, the methods involve in situ hybridization using directly or indirectly labelled molecules, e.g., complementary DNA or RNA or modified nucleic acids, as probes that bind or hybridize to an RNA substrate within a biological sample of interest. Nucleic acid probes or probe sets, in some examples, may be labelled with radioisotopes, epitopes, hapten, biotin, or fluorophores, to enable detection of the location of specific nucleic acid probes or probe sets in tissues.

In some embodiments, barcode sequences of the probes (or products thereof, such as amplification, extension, ligation, or hybridization products) are targeted by detectably labelled detection oligonucleotides, such as fluorescently labelled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751): 235-239 (2019); Chen et al., “Spatially resolved, highly multiplexed RNA profiling in single cells,” Science; 348(6233): aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, detection of the barcode sequences is performed by sequential hybridization of probes to the barcode sequences or complements thereof (e.g., in a generated RCA product). In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled probes that directly or indirectly hybridize to the barcode sequences or complements thereof (e.g., in amplification products generated using the polymerase fusion proteins provided herein), and dehybridizing the one or more detectably labeled probes. In any of the embodiments herein, the contacting and dehybridizing steps can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting step can comprise contacting the biological sample with one or more intermediate probes that directly or indirectly hybridize to the barcode sequences or complements thereof (e.g., in amplification products generated using the polymerase fusion proteins provided herein), wherein the one or more intermediate probes are detectable using one or more detectably labeled probes. In some embodiments, the method comprises detecting a rolling circle amplification product (RCP) generated using a circular or circularized primary probe or probe set as a template. In any of the embodiments herein, the detecting step can further comprise dehybridizing the one or more intermediate probes and/or the one or more detectably labeled probes from the barcode sequences or complements thereof. In any of the embodiments herein, the contacting and dehybridizing steps can be repeated with the one or more intermediate probes, the one or more detectably labeled probes, one or more other intermediate probes, and/or one or more other detectably labeled probes.

In some embodiments, a barcode sequence in a probe herein (e.g., a circular or circularizable probe) or a product thereof can be analyzed by in situ sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing by synthesis.

In some embodiments, the in situ hybridization comprises sequential fluorescent in situ hybridization. In some embodiments, the analysis and/or sequence determination comprises detecting a product generated by amplification using any of the Phi29 fusion proteins or compositions provided herein. In some embodiments, the detection or determination comprises hybridizing to the amplification product a detection oligonucleotide labelled with a fluorophore, an isotope, a mass tag, or a combination thereof. In some embodiments, the detection or determination comprises imaging the amplification product. In some embodiments, the detection or determination is performed when the RNA substrate is in situ in the tissue sample.

In some aspects, in situ assays use microscopy as a readout, e.g., nucleic acid sequencing, hybridization, or other detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, a provided method is quantitative and preserves the spatial information within a tissue sample without physically isolating cells or using homogenates.

In some aspects, the methods also involve imaging the amplification product (e.g., amplicon) and/or one or more portions of the polynucleotides, for example, via binding of a detectably labelled probe (e.g., to a sequence of circular or circularizable probe or a complement thereof) and detecting the detectable label. In some embodiments, the detectably labelled probe comprises a detectable label that can be measured and quantitated. A label or detectable label can be a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Autofluorescence can comprise background fluorescence (that can arise from a variety of sources, including aldehyde fixation, extracellular matrix components, red blood cells, lipofuscin, and the like), which is distinct from the desired immunofluorescence from the fluorescently labelled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

In some embodiments, a detectable probe containing a detectable label can be used to detect one or more polynucleotide(s) and/or amplification products (e.g., amplicon) described herein (e.g., a probe hybridized to an RNA substrate). In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as 1251, 35S, 14C, or 3H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labelled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. A fluorescent label can comprise a signalling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Nucleotides having other fluorophores can also be synthesized (See, Henegariu et al. (2000) Nature Biotechnol. 18:345), the content of which is herein incorporated by reference in its entirety.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labelled nucleotide and/or polynucleotide sequences (Lakowicz et al. (2003) Bio Techniques 34:62), the content of which is incorporated by reference in its entirety.

Biotin, or a derivative thereof, may also be used as a label on a nucleotide and/or a polynucleotide sequence, and subsequently bound by a detectably labelled avidin/streptavidin derivative (e.g., phycoerythrin-conjugated streptavidin), or a detectably labelled anti-biotin antibody. Digoxigenin may be incorporated as a label and subsequently bound by a detectably labelled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labelled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labelled, especially with a hapten that is then bound by a capture agent, e.g., as disclosed in U.S. Pat. Nos. 5,344,757, 5,702,888, 5,354,657, 5,198,537 and 4,849,336, and 5,192,782. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting comprises determining a signal, e.g., a fluorescent signal. In some aspects, the detection (comprising imaging) is carried out using any one of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a simpler set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possibly makes these types of microscopes particularly good at 3D imaging and surface profiling of samples. CLARITY™-optimized light sheet microscopy (COLM) provides an alternative microscopy for fast 3D imaging of large, clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequences can be analyzed in situ, e.g., by incorporation of a labelled nucleotide (e.g., fluorescently labelled mononucleotides or dinucleotides) in a sequential, template-dependent manner or hybridization of a labelled primer (e.g., a labelled random hexamer) to a nucleic acid template such that the identities (e.g., nucleotide sequence) of the incorporated nucleotides or labelled primer extension products can be determined, and consequently, the nucleotide sequence of the corresponding template nucleic acid. Aspects of in situ analysis are described, for example, in Mitra et al., (2003) Anal. Biochem. 320, 55-65, and Lee et al., (2014) Science, 343(6177), 1360-1363; US 2016/0024555; US 2019/0194709; U.S. Pat. Nos. 10,138,509; 10,494,662; 10,179,932, all of which are herein incorporated by reference in their entireties.

In some cases, sequencing can be performed after the analytes are released from the biological sample. In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, the method involves detecting the one or more barcode sequences in the circular or circularizable probe or a product thereof by contacting the biological sample that contains amplification products, with one or more detectably-labelled probes that directly or indirectly hybridize to the one or more barcode sequences, detecting signals associated with the one or more detectably-labelled probes, and dehybridizing the one or more detectably-labelled probes. In some embodiments, the contacting, detecting, and dehybridizing steps are repeated with the one or more detectably-labelled probes and/or one or more other detectably-labelled probes that directly or indirectly hybridize to the one or more barcode sequences. In some embodiments, the detectably labelled probes comprise a detectable label (e.g., are conjugated to a detectable label). In some embodiments, the detectably labelled probes are labelled with a sequence capable of hybridizing to a detection probe, wherein the detection probe comprises a detectable label (e.g., is conjugated to a detectable label). Methods of detecting and/or analyzing a sequence by sequential hybridization of probes have been described, for example, in U.S. Pat. Pub. 20210340618, the content of which is herein incorporated by reference in its entirety.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and U.S. Pat. No. 6,306,597, all of which are herein incorporated by reference in their entireties.

In some embodiments, nucleic acid hybridization can be used for sequence detection. These methods utilize labelled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

### V. Kits, Compositions, and Articles of Manufacture

Also provided herein are kits, compositions, and articles of manufacture, for example comprising a Phi29 fusion protein and one or more additional components (e.g., as described in Section II).

In some embodiments, provided herein is a composition comprising a Phi29 fusion protein disclosed herein (e.g., a fusion protein comprising a Phi29 polymerase, such as any described in Section II). In some embodiments, provided herein is a composition comprising a Phi29 fusion protein disclosed herein (e.g., a fusion protein comprising a Phi29 polymerase, such as any described in Section II).

In some aspects, provided herein is a composition comprising any one of the fusion proteins disclosed herein. In some aspects, provided herein is a composition comprising any one of the dimers disclosed herein. In some embodiments, at least 70%, 75%, 80%, 85%, 90%, 95%, 96%, 97%, 98%, 99% or 99.5% of the polypeptides or proteins of the composition is a fusion protein. In some embodiments, at least 70%, 75%, 80%, 85%, 90%, 95%, 96%, 97%, 98%, 99% or 99.5% of the polypeptides or proteins of the composition is a dimer.

In some embodiments, the composition has a pH of about 8.5. In some cases, the composition comprises between about 5 mM and about 10 mM MgCl2. In some cases, the composition comprises between about 0.1 mM and about 0.2 mM dNTPs. In some instances, the composition comprises between about 1 mM and about 4 mM DTT. In some cases, the composition comprises between 1 mg/mL and 3 mg/mL BSA (e.g., about 2 mg/mL BSA). In some cases, the composition comprises between 5 mM and 15 mM NH₄SO₄ (e.g., about 10 mM NH₄SO₄). In some embodiments the composition comprises between about 0.05 μM and about 1 μM of the recombinant polymerase or Phi29 fusion protein. In some cases, the composition comprises between about 0.5 μM and about 1 μM, between about 0.2 μM and about 1 μM, between about 0.8 μM and about 1 μM, or between about 0.05 UM and about 0.5 μM of the recombinant polymerase or Phi29 fusion protein. In some cases, the composition comprises at least 1, 10, 50, or 100 UM of the recombinant polymerase or Phi29 fusion protein. In some embodiments, the composition comprises between 10 and 50, between 10 and 100, between 50 and 100, between 25 and 50, or between 25 and 100 μM of the recombinant polymerase or Phi29 fusion protein. In any of the embodiments herein, the recombinant Phi29 fusion protein may be at least 85%, at least 90%, or at least 95% soluble in the composition. In any of the embodiments herein, the recombinant Phi29 fusion protein may be at least 85%, at least 90%, or at least 95% soluble in the composition at 20° C., 30° C., 37° C., 40° C., or 42° C. In some embodiments, the composition has a pH of about 8.5 and comprises 10 mM MgCl2, 10 mM NH₄SO₄, 4 mM DTT, and 0.2 mg/mL BSA. In some embodiments, the composition further comprises 0.2 mM dNTPs. In some embodiments, the composition has a pH of about 8.5 and comprises 10 mM MgCl2, 10 mM NH₄SO₄, 1 mM DTT, and 0.2 mg/mL BSA. In some embodiments, the composition further comprises 0.1 mM dNTPs. In some embodiments, the composition has a pH of about 8.5 and comprises 5 mM MgCl2, 10 mM NH₄SO₄, 1 mM DTT, and 0.2 mg/mL BSA. In some embodiments, the composition further comprises 0.2 mM dNTPs.

In some embodiments, a kit comprises one or more additional reagents for performing the methods provided herein, for example reagents required for one or more steps for amplification, detection, sequencing, and/or sample preparation as described herein. In some embodiments, the kit comprises a Phi29 fusion protein, for example, any described herein in Section II.E., and/or a polynucleotide, recombinant nucleic acid molecule, or vector, for example, as described in Section III.B. In some cases, the kit comprises any of the compositions disclosed herein.

In some embodiments, the kit comprises a buffer or a buffer stock solution (e.g., a solution that can be diluted to form a working buffer solution. In some embodiments, the kit comprises a buffer stock solution that can be diluted 2-fold, 3-fold, 4-fold, 5-fold, 10-fold, 20-fold, 50-fold, or 100-fold to produce a working buffer having a pH of about 8.5 and comprising 10 mM MgCl2, 10 mM NH₄SO₄, 4 mM DTT, and 0.2 mg/mL BSA. In some embodiments, the kit comprises a buffer stock solution that can be diluted 2-fold, 3-fold, 4-fold, 5-fold, 10-fold, 20-fold, 50-fold, or 100-fold to produce a working buffer having a pH of about 8.5 and comprising 10 mM MgCl2, 10 mM NH₄SO₄, 1 mM DTT, and 0.2 mg/mL BSA. In some embodiments, the kit comprises a buffer stock solution that can be diluted 2-fold, 3-fold, 4-fold, 5-fold, 10-fold, 20-fold, 50-fold, or 100-fold to produce a working buffer having a pH of about 8.5 and comprising 5 mM MgCl2, 10 mM NH₄SO₄, 1 mM DTT, and 0.2 mg/mL BSA. In some embodiments, the composition further comprises 0.2 mM dNTPs

In some embodiments, the kit further comprises one or more reagents for performing amplification. In some embodiments, the kit further comprises one or more components, such as a dNTP, a di-cation, and a reaction buffer. In some embodiments, the composition comprises MgCl2, dNTPs, DTT, BSA, NH4SO, or any combination thereof. In some embodiments, the kit further comprises a primer, a dNTP, a di-cation, and a reaction buffer. In some embodiments, the kit further comprises instructions for use, for example, instructions for amplifying a nucleic acid, e.g., using any of the Phi29 fusion proteins or compositions provided herein.

In some embodiments, the kit further comprises a detection probe for detection of a nucleic acid as described in Section IV, e.g., DNA or RNA. In some embodiments, the probe is a circular or circularizable probe for detection of the target nucleic acid, e.g., DNA or RNA.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection, such as barcode detection probes or detectable labels. In some embodiments, the kits optionally contain other components, for example nucleic acid primers, enzymes and reagents, buffers, nucleotides, modified nucleotides, reagents for additional assays.

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, provided herein is a kit comprising the Phi29 fusion proteins of Section II.E., the dimer of Section II.F., the composition of Section IV, the polynucleotide of Section III.A., the recombinant nucleic acid molecule of Section III.B., or the vector of Section III.B., and one or more of: (a) dNTPs; (b) one or more di-cation; (c) reaction buffer; (d) a buffer for use with any of (a)-(c); and (e) instructions for use. In some embodiments, the kit further comprises one or more primers. In some embodiments, the kit further comprises one or more circularizable probes. In some embodiments, the kit is for amplification of nucleic acid. In some embodiments, the kit further comprises a detection probe for detection of the amplified nucleic acid.

### VI. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in some ways to accomplish the same objectives.

It is understood that aspects and embodiments of the invention described herein, in addition to embodiments “comprising” certain aspects and features, may include “consisting of” and/or “consisting essentially of” aspects and embodiments. As used herein, the singular form “a”, “an”, and “the” includes plural references unless indicated otherwise. As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

In some aspects, conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa.

Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., U.S. Pat. No. 8,562,989; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20): 11882-6, the contents of each of which are herein incorporated by reference in their entireties).

An amino acid substitution may include replacement of one amino acid in a polypeptide with another amino acid. Amino acid substitutions may be introduced to generate a Phi29 fusion protein as described herein.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some contexts, conservative substitutions can involve the exchange of a member of one of these classes for another member of the same class. In some contexts, non-conservative amino acid substitutions can involve exchanging a member of one of these classes for another class. In some contexts, particular substitutions can be considered “conservative” or “non-conservative” depending on the stringency and context and environment of the particular residue in primary, secondary and/or tertiary structure of the protein.

The term “peptide” as used herein refers to a plurality of amino acids joined together, such as in a linear or circular chain. The term oligopeptide is typically used to describe peptides having between 2 and about 50 or more amino acids. Peptides larger than about 50 are often referred to as polypeptides or proteins.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An “isolated” protein, polypeptide or nucleic acid refers to a protein, polypeptide or nucleic acid molecule that has been separated from a component of its natural environment. An isolated protein or polypeptide can refer to a purified protein or polypeptide. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. An isolated protein or polypeptide (e.g., an isolated polymerase) includes a polymerase present in a composition comprising other molecules, including other proteins and/or nucleic acids, wherein the isolated protein or polypeptide has been separated from a component of its natural environment.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, a “recombinant expression system” comprises a host cell.

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from among adenine (A), thymine (T), cytosine (C), or guanine (G), and a ribonucleic acid can have one or more bases selected from among uracil (U), adenine (A), cytosine (C), or guanine (G). Useful non-native bases that can be included in a nucleic acid or nucleotide are known in the art.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

The term “polymerase” includes not only naturally-occurring enzymes but also all modified derivatives thereof, including also derivatives of naturally-occurring polymerase enzymes. For instance, in some embodiments, the polymerase can have been modified to remove 5′-3′ exonuclease activity. Sequence-modified derivatives or mutants of polymerase enzymes that can be used include, but are not limited to, mutants that retain at least some of the functional, e.g., polymerase activity of the wild-type sequence. Mutations can affect the activity profile of the enzymes, e.g., enhance or reduce the rate of polymerization, under different reaction conditions, e.g., temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 90N) DNA ligase (90NTM DNA ligase, available from New England Biolabs, Ipswich, MA), and Ampligase™ (available from Epicentre Biotechnologies, Madison, WI). Derivatives, e.g., sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g., reverse transcriptase, activity of the wild-type sequence. The reverse transcriptase enzyme can be provided as part of a composition that includes other components, e.g., stabilizing components that enhance or improve the activity of the reverse transcriptase enzyme, such as RNase inhibitor(s), inhibitors of DNA-dependent DNA synthesis, e.g., actinomycin D. Many sequence-modified derivative or mutants of reverse transcriptase enzymes, e.g., M-MLV, and compositions including unmodified and modified enzymes are commercially available, e.g., ArrayScript™, MultiScribe™, ThermoScript™, and SuperScript® I, II, III, and IV enzymes.

Certain reverse transcriptase enzymes (e.g., Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

In some embodiments, the quantification of RNA and/or DNA is carried out by real-time PCR (also known as quantitative PCR or qPCR), using techniques well known in the art, such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

The terms “detectable label,” “optical label,” and “label” are used interchangeably herein to refer to a directly or indirectly detectable moiety that is associated with (e.g., conjugated to) a molecule to be detected, e.g., a probe for in situ assay or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, a probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPYR FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1TM Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™(high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™Blue (pH 5), LysoSensor™ Green (pH 5), LysoSensor™Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARFR-1 (high pH), SNARFR-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTOR-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6, 7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and—methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof..

### VII. Examples

The following examples are included for illustrative purposes only and are not intended to limit the scope of the present disclosure.

### Example 1: In Vitro Rolling Circle Amplification (RCA) Assay Using Phi29 Fusion Proteins at Different Concentrations

This example describes the generation and use of exemplary Phi29 fusion proteins, including a Phi29 fusion protein with a dimerization domain and a Phi29 fusion protein with a heterologous nucleic acid binding domain, in rolling circle amplification (RCA).

An exemplary Phi29 fusion protein with a dimerization domain, Phi29-kinesin 1 dimerization domain (Phi29-dimer; set forth in SEQ ID NO:28), and an exemplary Phi29 fusion protein with a heterologous nucleic acid binding domain, Phi29-E. coli HU (Phi29-HU; set forth in SEQ ID NO:38), was recombinantly produced, and tested in an in vitro RCA assay. A circularizable probe (50 nM) was mixed with 100 nM of an RNA template and 50 nM of an RCA oligonucleotide, and incubated at 37° ° C. for 1 hour for probe hybridization to the template. 1 μM of a ligase was then added and incubated at 37ºC for 1 hour for ligation of the circularizable probe. 1 nM of ligated product was incubated with dNTPs and wild-type Phi29, Phi29-dimer or Phi29-HU at various concentrations (0 nM to 10,000 nM). SYBR Gold labelling of the RCPs was monitored in real-time, followed by reaction inactivation.

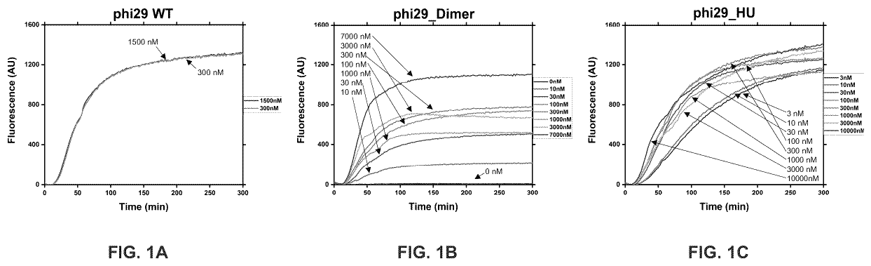

FIGS. 1A-1C show RCA product (RCP) formation for the various tested enzyme concentrations. Specifically, wild-type Phi29 polymerase (FIG. 1A) generated RCPs at a similar rate independent of enzyme concentration. Phi29-dimer (FIG. 1B) and Phi29-HU (FIG. 1C) generally exhibited concentration-dependent effects on RCP formation rate and signal intensity.

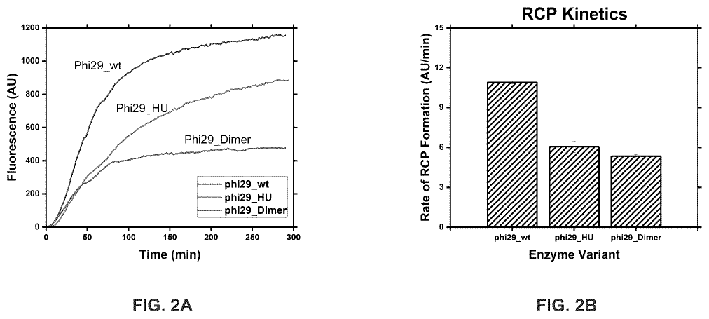

The kinetics of RCP generation of wild-type Phi29, Phi29-dimer and Phi29-HU were compared at a particular concentration (1 μM). As shown in FIGS. 2A-2B, wild-type Phi29 polymerase generated RCPs at a faster rate and in a higher total signal intensity compared to Phi29-kinesin 1 dimer or Phi29-HU. The results support that exemplary fusion proteins, including fusion proteins with a dimerization domain or a nucleic acid binding domain, can be employed in nucleic acid amplification methods such as RCA, to consistently produce RCP, in a concentration-dependent manner.

### Example 2: In Situ Rolling Circle Amplification (RCA) Assay Using Phi29 Fusion Proteins

This example describes the generation and use of exemplary Phi29 fusion proteins in an in situ rolling circle amplification (RCA) assay, demonstrating improved properties compared to a wild-type Phi29 polymerase.

The exemplary Phi29-dimer and Phi29-HU as described in Example 1 above were recombinantly produced, and evaluated in an in situ RCA assay. Briefly, a tissue sample was prepared for in situ RCA for gene expression analysis of three different transcripts (Transcripts 1-3) using the Phi29-dimer, the Phi29-HU or a wild-type Phi29 polymerase. Circularizable probes were added to each tissue sample and allowed to hybridize. Following probe hybridization, the tissue samples were washed to remove unbound probes. Circularizable probe ligation was performed by adding a ligase and a ligase buffer to the samples. For RCA, samples were incubated at 37° C. for 2 hours in a reaction mix containing a Phi29 polymerase buffer, dNTPs, and either wild-type Phi29 polymerase, Phi29-dimer or Phi29-HU. RCPs were detected by hybridizing labelled probes to the RCPs in situ and imaging the samples with a fluorescent microscope.

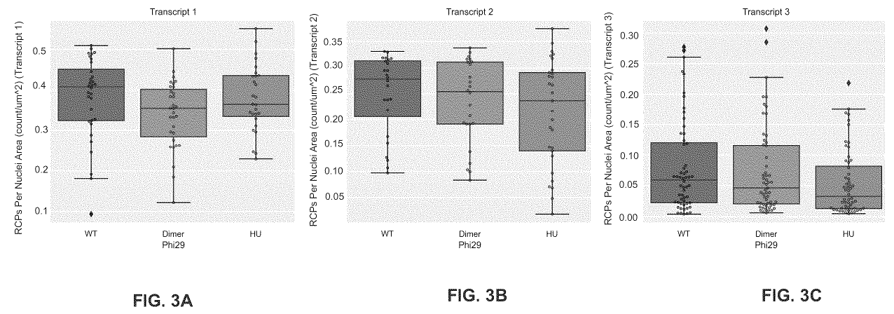

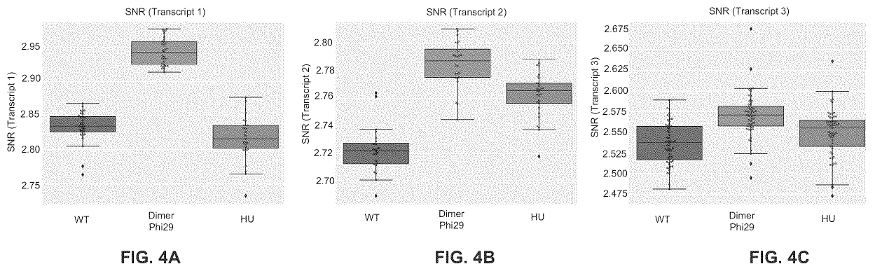

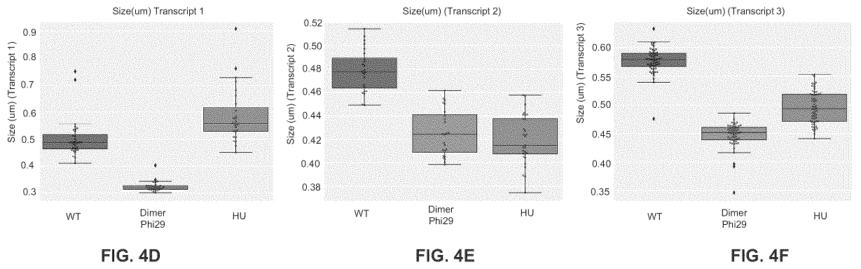

Results of the in situ RCA assay showed that all of the tested Phi29 polymerases or fusion proteins generated RCPs with high sensitivity, as shown by comparably high RCPs per nuclei area for Transcripts 1-3 (FIGS. 3A-3C). In general, the Phi29-dimer and the Phi29-HU, exhibited a higher signal-to-noise ratio compared to wild-type Phi29 for the three transcripts (FIGS. 4A-4C). This increase in signal-to-noise ratio also corresponded to generation of a smaller RCP size for both Phi29-dimer and Phi29-HU (FIGS. 4D-4F).

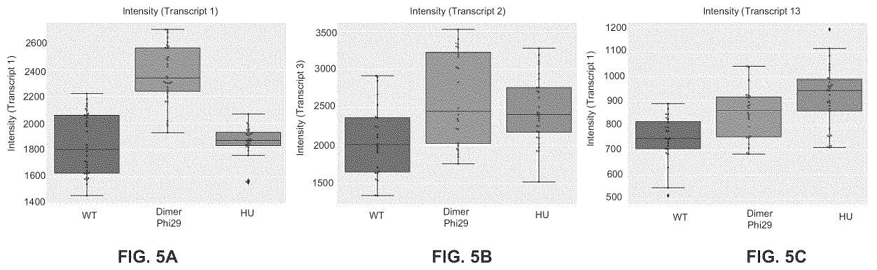

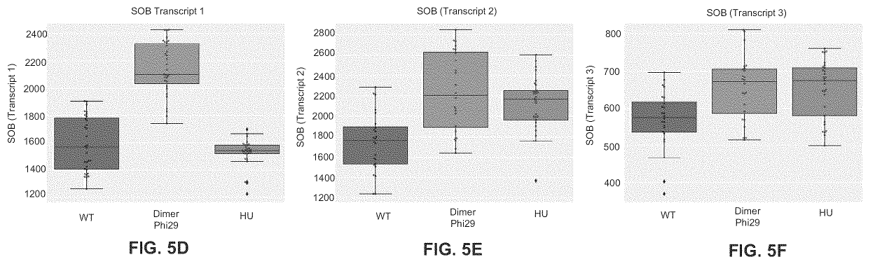

The signal intensity of the RCP generated by wild-type Phi29 polymerase, Phi29-dimer or Phi29-HU was determined. In general, the Phi29-dimer and the Phi29-HU exhibited a higher signal intensity (FIGS. 5A-5C) or signal-over-background of RCP detection, compared to wild-type Phi29 (FIGS. 5D-5F).

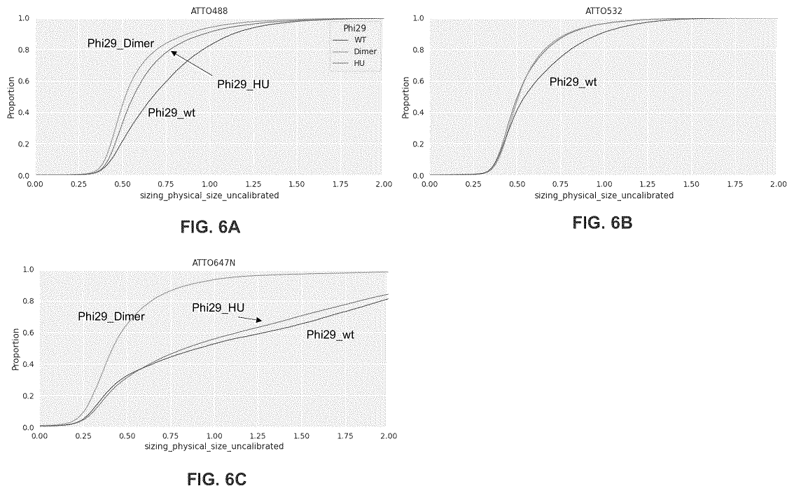

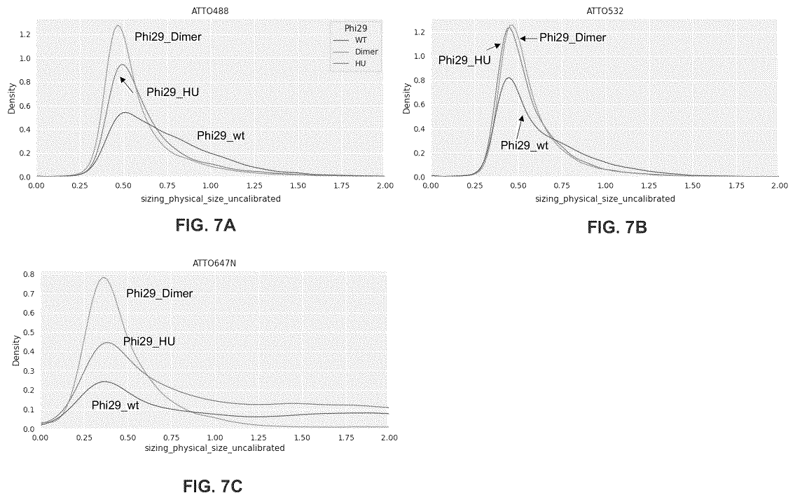

The sizes of the generated RCP were determined, and the proportion and density of RCPs over the sizes were plotted. FIGS. 6A-6C show the proportion of the sizes of the RCPs as determined in the assay for Transcripts 1-3. As shown, RCPs generated by Phi29-dimer and Phi29-HU were generally smaller. These results were consistent with the results as shown above in FIGS. 4D-4F showing that Phi29-dimer and Phi29-HU generated smaller RCPs compared to wild-type Phi29. Additionally, the RCPs generated by Phi29-dimer or Phi29-HU exhibited more uniform and narrower size distribution on the curve, as shown in FIGS. 7A-7C.

The results showed that exemplary Phi29 polymerase fusion proteins, Phi29-dimer and Phi29-HU, exhibit improved properties observed in an in situ RCA assay compared to a wild-type Phi29 polymerase. These results demonstrate higher signal intensity, signal-over-background, more uniform size distribution and smaller size of RCPs in an in situ RCA assay, and are consistent with improved processivity and/or affinity for circular DNA-primer structures. Accordingly, the results support the utility and benefit of exemplary fusion Phi29 polymerases in methods of nucleic acid amplification, such as in situ RCA.

### Example 3: Solubility Assessment of Spycatcher and Spytag-Containing Fusion Proteins

This example describes the generation and protein solubility assessment of two different fusion proteins comprising a Phi29 polymerase: (1) a recombinant Phi29 polymerase-SpyCatcher fusion protein (SEQ ID NO:34) having an amino terminal polyhistidine/SUMO tag (N-His/SUMO) and (2) a recombinant Phi29 polymerase-SpyTag fusion protein (SEQ ID NO:36) having an amino terminal polyhistidine/SUMO tag (N-His/SUMO).

E. coli were transformed with plasmid vectors encoding the N-His/SUMO SpyCatcher or SpyTag and expression of the recombinant polymerase was induced.

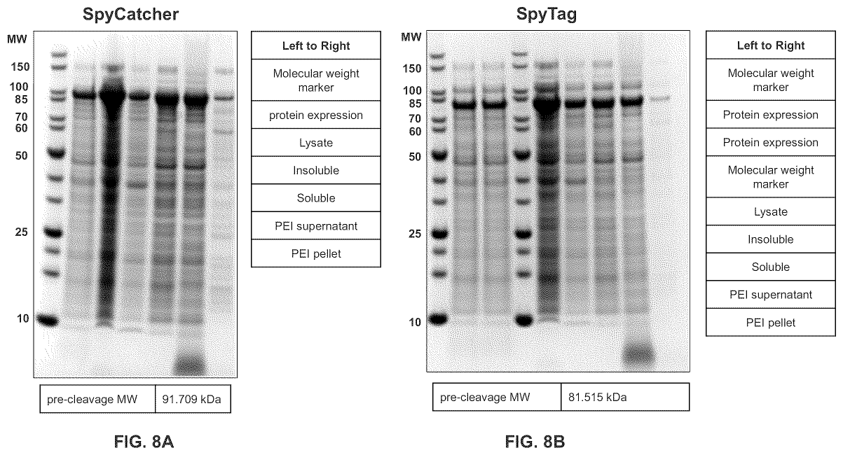

The culture was centrifuged, and the cell pellet was resuspended in lysis buffer. The lysate was centrifuged at a high speed to obtain a soluble and insoluble fraction. Samples were run on an SDS-PAGE for protein solubility analysis, as shown in FIGS. 8A-8B.

As shown in FIG. 8A, the N-His/SUMO Phi29 polymerase-SpyCatcher fusion protein produced a band of approximately 92 kDa. Additionally, the majority of the Phi29 polymerase-SpyCatcher fusion protein remained in the PEI supernatant (lane 5 on blot), rather than the PEI pellet (lane 6 on blot), indicating that the majority of the Phi29 polymerase-SpyCatcher fusion protein was stable.

As shown in FIG. 8B, the N-His/SUMO Phi29 polymerase-SpyTag fusion protein produced a band of approximately 82 kDa. Additionally, the majority of recombinant Spy Tag polypeptide remained in the PEI supernatant (lane 8 on blot), rather than the PEI pellet (lane 9 on blot), indicating that the majority of SpyCatcher was stable.

### Example 4: Purification, Characterization and Coupling of SpyCatcher and SpyTag-containing Fusion Proteins

This example demonstrates purification and characterization of the Phi29 polymerase-SpyCatcher fusion protein, the Phi29 polymerase-SpyTag fusion protein, and a dimer generated from the two fusion proteins after protein coupling.

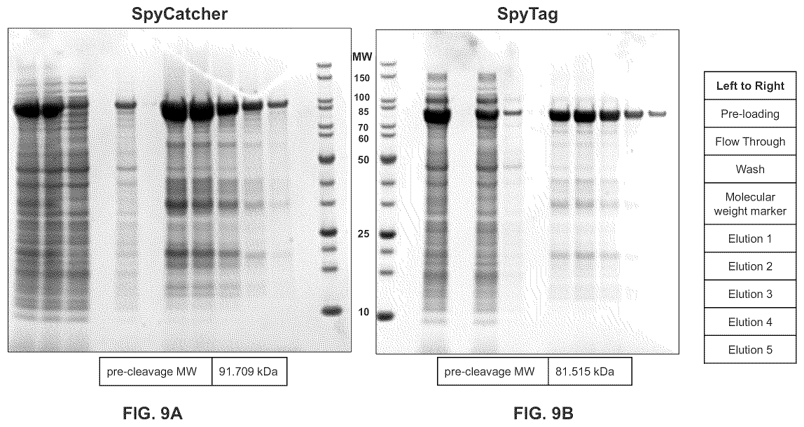

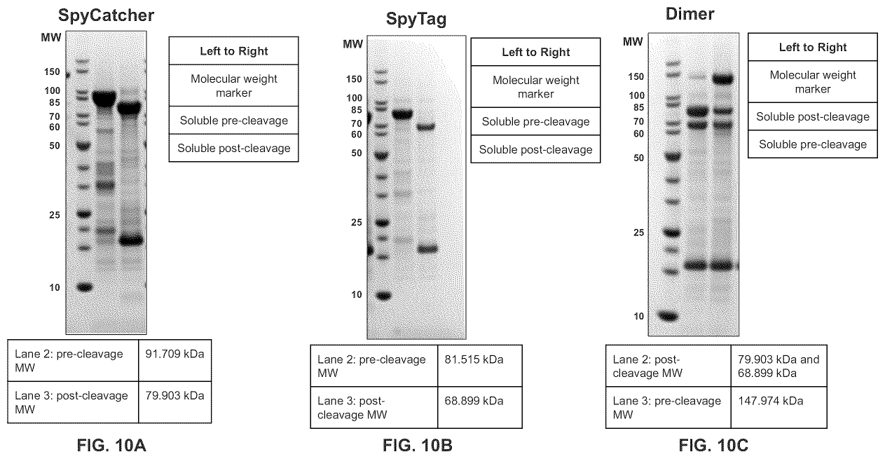

N-His/SUMO recombinant Phi29 polymerase-SpyCatcher fusion protein and recombinant Phi29 polymerase-SpyTag fusion protein were expressed as described in Example 3. The soluble fraction was collected and run on a nickel column. The SDS-PAGE for the various fractions are shown in FIG. 9A (SpyCatcher) and FIG. 9B (SpyTag). The SUMO tag was cleaved using a recombinant Ubl-specific protease 1 (ULP1). Fractions were collected for pre- and post-cleavage of the N-His/SUMO tag, as shown in FIG. 10A (SpyCatcher) and FIG. 10B (SpyTag). Each elution fraction was collected as 1 mL aliquots using an 80-100% gradient of 1 M NaCl. As shown in FIGS. 10A-10B, eluted fractions of the cleaved N-His/SUMO Phi29 polymerase-SpyCatcher fusion protein produced a doublet band, with the Phi29 polymerase-SpyCatcher fusion protein running at approximately 80 kDa (lane 3 on blot), indicating that the doublet is retained with nickel purification. Eluted fractions of the N-His/SUMO Phi29 polymerase-SpyTag fusion protein produced a doublet band, with the Phi29 polymerase-SpyTag fusion protein running at approximately 69 kDa (lane 3 on blot), indicating that the doublet is retained with nickel purification.

When the Phi29 polymerase-SpyCatcher fusion protein, the Phi29 polymerase-SpyTag fusion protein were co-expressed in E. coli, a dimer of fusion proteins (e.g., a dimer of Phi29 polymerase-SpyCatcher:Phi29 polymerase-SpyTag; comprising two Phi29 polymerase domains), with an approximate molecular weight of 147.974 kDa, was generated based on the protein coupling between SpyCatcher:SpyTag. (FIG. 10C, lane 3). Eluted fractions of the cleaved Phi29 polymerase-SpyCatcher: Phi29 polymerase-Spy Tag produced a triplet band, with the Phi29 polymerase-SpyCatcher fusion protein running at approximately 80 kDa (lane 2) and the Phi29 polymerase-SpyTag fusion protein running at approximately 69 kDa (lane 2), indicating that the triplet is retained with nickel purification.

Minor impurities at lower molecular weights were present at low concentrations and could be removed by an additional Heparin chromatography step, or by using a 100 kDa MWCO concentrator (data not shown).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID NO. | SEQUENCE | DESCRIPTION |
| 1. | MKHMPRKMYSCDFETTTKVEDCRVWAYGYMNIEDHSEYKIGNSLDEFMAWV | WT Phi29 |
| | LKVQADLYFHNLKEDGAFIINWLERNGFKWSADGLPNTYNTIISRMGQWYM | Polymerase |
| | IDICLGYKGKRKIHTVIYDSLKKLPFPVKKIAKDFKLTVLKGDIDYHKERP | with initial |
| | VGYKITPEEYAYIKNDIQIIAEALLIQFKQGLDRMTAGSDSLKGFKDIITT | MK (aa) |
| | KKFKKVFPTLSLGLDKEVRYAYRGGFTWLNDRFKEKEIGEGMVEDVNSLYP | |
| | AQMYSRLLPYGEPIVFEGKYVWDEDYPLHIQHIRCEFELKEGYIPTIQIKR | |
| | SRFYKGNEYLKSSGGEIADLWLSNVDLELMKEHYDLYNVEYISGLKFKATT | |
| | GLFKDFIDKWTYIKTTSEGAIKQLAKLMLNSLYGKFASNPDVTGKVPYLKE | |
| | NGALGFRLGEEETKDPVYTPMGVFITAWARYTTITAAQACYDRIIYCDTDS | |
| | IHLTGTEIPDVIKDIVDPKKLGYWAHESTFKRAKYLRQKTYIQDIYMKEVD | |
| | GKLVEGSPDDYTDIKFSVKCAGMTDKIKKEVTFENEKVGFSRKMKPKPVQV | |
| | PGGVVLVDDTFTIK | |
| 2. | ATGAAGCACATGCCACGTAAAATGTATAGCTGTGATTTTGAGACTACGACG | WT Phi29 |
| | AAAGTTGAAGATTGCCGTGTGTGGGCGTATGGTTACATGAATATTGAAGAT | Polymerase |
| | CACTCCGAGTATAAGATTGGCAATAGCCTGGATGAATTCATGGCGTGGGTG | with initial |
| | CTGAAGGTTCAGGCCGATTTGTACTTTCATAATCTGAAATTTGATGGCGCT | MK (nt) |
| | TTTATCATTAACTGGCTGGAGCGTAATGGTTTCAAGTGGAGCGCAGATGGC | |
| | CTGCCGAATACGTACAACACCATTATCAGCCGTATGGGCCAGTGGTACATG | |
| | ATCGACATCTGCCTGGGCTATAAGGGTAAGCGTAAGATCCACACCGTGATT | |
| | TATGACTCCCTGAAAAAGCTGCCGTTTCCGGTGAAGAAAATTGCCAAAGAC | |
| | TTCAAGCTGACGGTTCTGAAGGGCGACATCGATTATCACAAGGAACGTCCG | |
| | GTTGGTTACAAGATCACCCCGGAAGAATACGCGTACATCAAAAACGACATT | |
| | CAAATCATCGCAGAGGCCTTGCTGATTCAGTTCAAGCAAGGTCTGGACCGC | |
| | ATGACTGCCGGTAGCGATTCCCTGAAGGGTTTCAAAGACATTATCACCACC | |
| | AAAAAGTTCAAAAAAGTGTTCCCGACGCTGAGCCTGGGTCTGGATAAAGAG | |
| | GTCCGTTACGCTTATCGTGGCGGCTTCACCTGGTTGAACGATCGTTTCAAA | |
| | GAAAAAGAAATTGGTGAGGGCATGGTTTTTGATGTTAACTCACTGTACCCG | |
| | GCACAAATGTATAGCCGCCTGCTGCCGTATGGCGAGCCGATCGTGTTCGAG | |
| | GGTAAATACGTGTGGGACGAAGATTACCCGCTGCATATTCAACATATCCGC | |
| | TGCGAGTTCGAGCTGAAGGAAGGCTACATCCCGACCATTCAGATTAAGCGT | |
| | AGCCGTTTTTACAAAGGTAATGAATACTTGAAGTCCTCGGGCGGTGAGATT | |
| | GCGGATCTGTGGTTAAGCAACGTCGACCTTGAGCTGATGAAAGAGCACTAT | |
| | GACCTCTATAACGTTGAGTACATTAGCGGTCTGAAATTCAAAGCCACGACG | |
| | GGTCTGTTTAAAGACTTCATTGACAAGTGGACCTATATCAAGACGACGAGC | |
| | GAGGGCGCGATCAAACAGCTGGCGAAGCTGATGCTGAATTCTCTGTACGGT | |
| | AAGTTTGCTAGCAATCCAGATGTCACCGGCAAAGTGCCGTATCTGAAAGAA | |
| | AACGGTGCGTTGGGTTTTCGCCTGGGTGAAGAGGAAACCAAAGATCCGGTG | |
| | TACACCCCGATGGGCGTTTTCATTACCGCGTGGGCTCGTTACACCACGATT | |
| | ACCGCAGCGCAGGCATGTTATGACCGTATCATTTACTGTGATACGGATAGC | |
| | ATTCACTTGACCGGTACCGAGATCCCAGATGTCATCAAAGATATTGTCGAC | |
| | CCGAAGAAACTGGGTTACTGGGCCCACGAAAGCACTTTCAAACGCGCAAAG | |
| | TATCTGCGTCAGAAAACGTACATCCAAGACATTTACATGAAAGAGGTTGAC | |
| | GGCAAATTGGTCGAGGGTTCGCCGGACGACTACACCGACATCAAGTTCAGC | |
| | GTGAAGTGCGCGGGTATGACCGACAAAATCAAAAAAGAAGTCACCTTTGAG | |
| | AACTTTAAGGTTGGTTTTAGCCGCAAGATGAAGCCTAAACCGGTTCAGGTC | |
| | CCGGGTGGCGTTGTGCTGGTCGACGACACCTTTACTATCAAG | |
| 3. | KHMPRKMYSCDFETTTKVEDCRVWAYGYMNIEDHSEYKIGNSLDEFMAWVL | WT Phi29 |
| | KVQADLYFHNLKEDGAFIINWLERNGFKWSADGLPNTYNTIISRMGQWYMI | Polymerase |
| | DICLGYKGKRKIHTVIYDSLKKLPFPVKKIAKDFKLTVLKGDIDYHKERPV | with initial |
| | GYKITPEEYAYIKNDIQIIAEALLIQFKQGLDRMTAGSDSLKGFKDIITTK | K (aa) |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIK | |
| 4. | AAGCACATGCCACGTAAAATGTATAGCTGTGATTTTGAGACTACGACGAAA | WT Phi29 |
| | GTTGAAGATTGCCGTGTGTGGGCGTATGGTTACATGAATATTGAAGATCAC | Polymerase |
| | TCCGAGTATAAGATTGGCAATAGCCTGGATGAATTCATGGCGTGGGTGCTG | with initial |
| | AAGGTTCAGGCCGATTTGTACTTTCATAATCTGAAATTTGATGGCGCTTTT | K (nt) |
| | ATCATTAACTGGCTGGAGCGTAATGGTTTCAAGTGGAGCGCAGATGGCCTG | |
| | CCGAATACGTACAACACCATTATCAGCCGTATGGGCCAGTGGTACATGATC | |
| | GACATCTGCCTGGGCTATAAGGGTAAGCGTAAGATCCACACCGTGATTTAT | |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAG | |
| 5. | MSKHMPRKMYSCDFETTTKVEDCRVWAYGYMNIEDHSEYKIGNSLDEFMAW | WT Phi29 |
| | VLKVQADLYFHNLKEDGAFIINWLERNGFKWSADGLPNTYNTIISRMGQWY | Polymerase |
| | MIDICLGYKGKRKIHTVIYDSLKKLPFPVKKIAKDFKLTVLKGDIDYHKER | with initial |
| | PVGYKITPEEYAYIKNDIQIIAEALLIQFKQGLDRMTAGSDSLKGFKDIIT | MS (aa) |
| | TKKFKKVFPTLSLGLDKEVRYAYRGGFTWLNDRFKEKEIGEGMVEDVNSLY | |
| | PAQMYSRLLPYGEPIVFEGKYVWDEDYPLHIQHIRCEFELKEGYIPTIQIK | |
| | RSRFYKGNEYLKSSGGEIADLWLSNVDLELMKEHYDLYNVEYISGLKFKAT | |
| | TGLFKDFIDKWTYIKTTSEGAIKQLAKLMLNSLYGKFASNPDVTGKVPYLK | |
| | ENGALGFRLGEEETKDPVYTPMGVFITAWARYTTITAAQACYDRIIYCDTD | |
| | SIHLTGTEIPDVIKDIVDPKKLGYWAHESTEKRAKYLRQKTYIQDIYMKEV | |
| | DGKLVEGSPDDYTDIKFSVKCAGMTDKIKKEVTFENFKVGFSRKMKPKPVQ | |
| | VPGGVVLVDDTFTIK | |
| 6. | SKHMPRKMYSCDFETTTKVEDCRVWAYGYMNIEDHSEYKIGNSLDEFMAWV | WT Phi29 |
| | LKVQADLYFHNLKEDGAFIINWLERNGFKWSADGLPNTYNTIISRMGQWYM | Polymerase |
| | IDICLGYKGKRKIHTVIYDSLKKLPFPVKKIAKDFKLTVLKGDIDYHKERP | with initial |
| | VGYKITPEEYAYIKNDIQIIAEALLIQFKQGLDRMTAGSDSLKGFKDIITT | S (aa) |
| | KKFKKVFPTLSLGLDKEVRYAYRGGFTWLNDRFKEKEIGEGMVEDVNSLYP | |
| | AQMYSRLLPYGEPIVFEGKYVWDEDYPLHIQHIRCEFELKEGYIPTIQIKR | |
| | SRFYKGNEYLKSSGGEIADLWLSNVDLELMKEHYDLYNVEYISGLKFKATT | |
| | GLFKDFIDKWTYIKTTSEGAIKQLAKLMLNSLYGKFASNPDVTGKVPYLKE | |
| | NGALGFRLGEEETKDPVYTPMGVFITAWARYTTITAAQACYDRIIYCDTDS | |
| | IHLTGTEIPDVIKDIVDPKKLGYWAHESTFKRAKYLRQKTYIQDIYMKEVD | |
| | GKLVEGSPDDYTDIKFSVKCAGMTDKIKKEVTFENEKVGFSRKMKPKPVQV | |
| | PGGVVLVDDTFTIK | |
| 7. | KHMPRKMYSCDFETTTKVEDCRVWAYGYMNIEDHSEYKIGNSLDEFMAWVL | Phi29 A68S |
| | KVQADLYFHNLKFDGSFIINWLERNGFKWSEDGLPNTYNTIISRMGQWYMI | A83E (aa) |
| | DICLGYKGKRKIHTVIYDSLKKLPFPVKKIAKDFKLTVLKGDIDYHKERPV | |
| | GYKITPEEYAYIKNDIQIIAEALLIQFKQGLDRMTAGSDSLKGFKDIITTK | |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTEKRAKYLRQKTYIQDIYMKEVDG | |
| | KLVEGSPDDYTDIKFSVKCAGMTDKIKKEVTFENEKVGFSRKMKPKPVQVP | |
| | GGVVLVDDTFTIK | |
| 8. | AAGCACATGCCACGTAAAATGTATAGCTGTGATTTTGAGACTACGACGAAA | Phi29 A68S |
| | GTTGAAGATTGCCGTGTGTGGGCGTATGGTTACATGAATATTGAAGATCAC | A83E (nt) |
| | TCCGAGTATAAGATTGGCAATAGCCTGGATGAATTCATGGCGTGGGTGCTG | |
| | AAGGTTCAGGCCGATTTGTACTTTCATAATCTGAAATTTGATGGCAGCTTT | |
| | ATCATTAACTGGCTGGAGCGTAATGGTTTCAAGTGGAGCGAAGATGGCCTG | |
| | CCGAATACGTACAACACCATTATCAGCCGTATGGGCCAGTGGTACATGATC | |
| | GACATCTGCCTGGGCTATAAGGGTAAGCGTAAGATCCACACCGTGATTTAT | |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAG | |
| 9. | GGS | GGS linker |
| | | (aa) |
| 10. | GGSGGS | GGSGGS |
| | | linker (aa) |
| 11. | GGTGGCAGCGGTGGCAGC | GGSGGS |
| | | linker (nt) |
| 12. | GGSGG | GGSGG linker |
| | | (aa) |
| 13. | GGGGG | GGGGG linker |
| | | (aa) |
| 14. | GGAGG | GGAGG linker |
| | | (aa) |
| 15. | GGGGSSS | GGGGSSS |
| | | linker (aa) |
| 16. | GGGGAAA | GGGGAAA |
| | | linker (aa) |
| 17. | GGGGS | (GGGGS) n |
| | | linker (aa), |
| | | n is 1 to 10 |
| 18. | KTVKNVVCVNEELTAEEWKRRYEKEKEKNARLKGKVEKLEIELARWRAGET | Kinesin-1 |
| | VKAEEQINMEDLMEASTPNLEVEAAQTAAAEAALAAQRTALANMSASVAVN | dimerization |
| | EQARLATECERLYQQLDDKDEEINQQSQYAEQLKEQVMEQEELIANARREY | domain- |
| | ETLQSEMARIQQENESAKEEVKEVLQALEELAVNYDQKSQEIDNKNKDIDA | short (aa) |
| | LNEELQQKQSVFNAASTELQQLKDMS | |
| 19. | AAGACAGTAAAGAACGTTGTATGTGTCAATGAGGAACTGACCGCCGAAGAG | Kinesin-1 |
| | TGGAAACGCCGCTACGAGAAAGAGAAAGAGAAGAATGCGCGTTTAAAGGGT | dimerization |
| | AAGGTGGAGAAACTGGAAATCGAATTAGCTCGTTGGCGCGCTGGCGAGACC | domain- |
| | GTGAAAGCCGAAGAGCAAATCAATATGGAAGATCTTATGGAAGCCTCAACT | short (nt) |
| | CCTAATCTGGAAGTGGAAGCGGCTCAAACCGCAGCAGCAGAGGCTGCACTG | |
| | GCGGCCCAGCGCACCGCACTCGCAAACATGTCTGCGTCCGTGGCGGTAAAT | |
| | GAACAGGCACGTCTGGCAACCGAATGTGAACGCCTTTATCAACAATTGGAC | |
| | GACAAAGACGAGGAGATCAACCAACAAAGTCAGTACGCAGAGCAATTGAAA | |
| | GAACAAGTTATGGAGCAAGAAGAGTTGATTGCGAATGCGCGCCGTGAGTAC | |
| | GAGACTCTTCAGAGTGAGATGGCCCGTATTCAGCAAGAAAATGAGAGCGCG | |
| | AAGGAAGAGGTAAAGGAAGTCTTACAAGCACTGGAAGAGCTTGCAGTGAAT | |
| | TACGATCAAAAGAGCCAGGAGATCGACAATAAGAATAAAGACATCGACGCC | |
| | CTGAACGAGGAGCTGCAGCAGAAACAATCGGTTTTCAACGCGGCGTCAACC | |
| | GAGTTGCAACAACTTAAAGATATGTCT | |
| 20. | KTVKNVVCVNEELTAEEWKRRYEKEKEKNARLKGKVEKLEIELARWRAGET | Kinesin-1 |
| | VKAEEQINMEDLMEASTPNLEVEAAQTAAAEAALAAQRTALANMSASVAVN | dimerization |
| | EQARLATECERLYQQLDDKDEEINQQSQYAEQLKEQVMEQEELIANARREY | domain- |
| | ETLQSEMARIQQENESAKEEVKEVLQALEELTVNYDQKSQEIDNKNKDIDA | long (aa) |
| | LNEELQQKQSVFNAASTELQQLKMLVPRGPVHRLGDPPVAT | |
| 21. | RTALANMSASVAVNEQARLATECERLYQQLDDKDEEINQQSQYAEQLKEQV | Kinesin-1 |
| | M EQEELIANARREYETLQSEMAR | delta-tail |
| | | (aa) |
| 22. | VTTLSGLSGEQGPSGDMTTEEDSATHIKESKRDEDGRELAGATMELRDSSG | Spycatcher00 |
| | KTISTWISDGHVKDFYLYPGKYTFVETAAPDGYEVATPIEFTVNEDGQVTV | 3 (aa) |
| | DGEATEGDAHT | |
| 23. | GTGACTACTTTATCCGGTTTGTCTGGAGAGCAAGGGCCATCCGGGGATATG | Spycatcher00 |
| | ACCACAGAAGAGGATTCAGCCACACACATCAAGTTCTCAAAACGCGACGAA | 3 (nt) |
| | GATGGCCGTGAATTGGCGGGTGCGACTATGGAACTTCGTGACTCATCCGGC | |
| | AAGACGATTAGCACATGGATTAGTGATGGTCATGTGAAAGACTTTTATCTG | |
| | TACCCAGGGAAGTACACTTTTGTCGAGACCGCTGCTCCCGATGGTTACGAA | |
| | GTCGCTACGCCTATTGAATTTACTGTTAATGAAGATGGTCAGGTGACGGTC | |
| | GATGGAGAAGCGACAGAGGGCGATGCGCACACT | |
| 24. | RGVPHIVMVDAYKRYK | SpyTag003 |
| | | (aa) |
| 25. | CGCGGAGTTCCACACATCGTCATGGTGGATGCGTATAAGCGTTACAAG | SpyTag003 |
| | | (nt) |
| 26. | MNKTQLIDVIAEKAELSKTQAKAALESTLAAITESLKEGDAVQLVGFGTEK | HU domain |
| | VNHRAERTGRNPQTGKEIKIAAANVPAFVSGKALKDAVK | (aa) |
| 27. | ATGAATAAGACGCAACTGATTGACGTCATTGCAGAGAAAGCCGAGTTGAGC | HU domain |
| | AAGACCCAGGCGAAAGCCGCGCTGGAGAGCACGCTGGCAGCGATTACCGAG | (nt) |
| | AGCCTGAAAGAGGGCGATGCAGTTCAACTGGTTGGCTTCGGCACCTTCAAG | |
| | GTCAACCACCGTGCGGAGCGCACCGGTCGCAACCCGCAGACCGGTAAAGAA | |
| | ATCAAGATTGCAGCCGCGAACGTGCCGGCGTTCGTCAGCGGTAAGGCACTG | |
| | AAAGATGCAGTAAAG | |
| 28. | KHMPRKMYSCDFETTTKVEDCRVWAYGYMNIEDHSEYKIGNSLDEFMAWVL | Phi29 with |
| | KVQADLYFHNLKFDGAFIINWLERNGFKWSADGLPNTYNTIISRMGQWYMI | K-linker- |
| | DICLGYKGKRKIHTVIYDSLKKLPFPVKKIAKDFKLTVLKGDIDYHKERPV | Kinesin-1 |
| | GYKITPEEYAYIKNDIQIIAEALLIQFKQGLDRMTAGSDSLKGFKDIITTK | dimerization |
| | KFKKVFPTLSLGLDKEVRYAYRGGFTWINDRFKEKEIGEGMVEDVNSLYPA | domain short |
| | QMYSRLLPYGEPIVFEGKYVWDEDYPLHIQHIRCEFELKEGYIPTIQIKRS | (aa) |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELAVNY | |
| | DQKSQEIDNKNKDIDALNEELQQKQSVENAASTELQQLKDMS | |
| 29. | AAGCACATGCCTCGCAAGATGTACAGCTGCGATTTTGAGACCACTACAAAG | Phi29 with |
| | GTTGAAGACTGCCGTGTCTGGGCGTATGGGTATATGAATATTGAAGACCAC | K-linker- |
| | AGCGAGTACAAGATTGGTAACAGCTTGGACGAATTCATGGCTTGGGTTCTC | Kinesin-1 |
| | AAGGTTCAAGCAGATCTTTATTTCCATAACCTCAAGTTTGATGGCGCGTTT | dimerization |
| | ATCATTAACTGGTTGGAGCGTAATGGATTTAAGTGGAGTGCCGACGGTTTG | domain short |
| | CCGAACACCTATAACACGATCATCTCTCGCATGGGTCAATGGTATATGATC | (nt) |
| | GACATTTGTCTGGGCTACAAGGGCAAGCGTAAGATTCATACAGTCATCTAC | |
| | GACTCACTTAAGAAATTGCCTTTCCCTGTTAAGAAGATCGCGAAAGACTTC | |
| | AAGTTGACCGTTCTTAAGGGCGACATTGACTATCACAAAGAGCGTCCGGTG | |
| | GGATATAAAATCACTCCAGAAGAGTACGCTTACATCAAGAACGACATCCAG | |
| | ATTATCGCAGAGGCGTTACTCATCCAGTTCAAACAAGGGCTGGATCGTATG | |
| | ACCGCTGGCTCAGACAGTTTAAAGGGCTTCAAAGACATTATCACTACGAAG | |
| | AAATTCAAGAAGGTCTTTCCTACCTTGAGTCTCGGACTGGACAAAGAGGTG | |
| | CGTTACGCCTATCGCGGCGGTTTCACTTGGCTTAACGACCGTTTCAAAGAG | |
| | AAAGAGATTGGCGAAGGTATGGTGTTCGACGTGAATAGTCTCTACCCGGCG | |
| | CAGATGTATTCCCGCTTGTTACCGTACGGTGAACCGATTGTGTTCGAGGGG | |
| | AAGTATGTCTGGGACGAAGACTACCCTTTACACATCCAGCATATTCGTTGC | |
| | GAGTTTGAGTTAAAAGAGGGTTACATCCCGACCATTCAAATTAAGCGTAGT | |
| | CGTTTCTATAAAGGGAATGAGTACTTGAAGTCTAGCGGCGGCGAGATTGCT | |
| | GACCTTTGGCTTTCAAACGTCGACCTGGAGCTTATGAAAGAGCACTATGAT | |
| | TTGTACAACGTGGAATATATCAGCGGGCTCAAGTTCAAAGCGACCACAGGG | |
| | TTATTCAAGGATTTCATTGATAAGTGGACTTATATCAAAACGACGAGTGAG | |
| | GGCGCAATCAAACAATTGGCGAAGCTCATGCTTAACAGCTTGTACGGAAAG | |
| | TTTGCTTCGAACCCAGATGTAACAGGCAAGGTCCCGTACCTCAAAGAAAAT | |
| | GGCGCATTAGGCTTCCGCCTGGGTGAAGAGGAAACGAAAGACCCTGTTTAT | |
| | ACCCCTATGGGCGTATTTATTACCGCCTGGGCGCGCTACACAACTATTACC | |
| | GCGGCGCAGGCCTGCTATGACCGTATTATTTACTGCGACACCGACAGCATC | |
| | CACCTCACCGGGACGGAGATCCCAGACGTCATCAAGGATATTGTGGACCCG | |
| | AAGAAGTTGGGCTATTGGGCGCATGAGTCAACCTTTAAACGTGCCAAATAT | |
| | CTGCGTCAAAAGACCTACATCCAAGATATCTATATGAAGGAAGTTGACGGT | |
| | AAGTTGGTTGAAGGTAGTCCGGACGATTATACAGACATTAAATTTAGCGTA | |
| | AAGTGCGCGGGCATGACAGACAAAATTAAGAAGGAAGTAACGTTCGAAAAT | |
| | TTTAAGGTTGGTTTCTCGCGCAAGATGAAGCCTAAACCGGTGCAAGTCCCT | |
| | GGTGGGGTTGTCCTTGTTGATGACACCTTTACCATTAAGGGTGGTTCCAAG | |
| | ACAGTAAAGAACGTTGTATGTGTCAATGAGGAACTGACCGCCGAAGAGTGG | |
| | AAACGCCGCTACGAGAAAGAGAAAGAGAAGAATGCGCGTTTAAAGGGTAAG | |
| | GTGGAGAAACTGGAAATCGAATTAGCTCGTTGGCGCGCTGGCGAGACCGTG | |
| | AAAGCCGAAGAGCAAATCAATATGGAAGATCTTATGGAAGCCTCAACTCCT | |
| | AATCTGGAAGTGGAAGCGGCTCAAACCGCAGCAGCAGAGGCTGCACTGGCG | |
| | GCCCAGCGCACCGCACTCGCAAACATGTCTGCGTCCGTGGCGGTAAATGAA | |
| | CAGGCACGTCTGGCAACCGAATGTGAACGCCTTTATCAACAATTGGACGAC | |
| | AAAGACGAGGAGATCAACCAACAAAGTCAGTACGCAGAGCAATTGAAAGAA | |
| | CAAGTTATGGAGCAAGAAGAGTTGATTGCGAATGCGCGCCGTGAGTACGAG | |
| | ACTCTTCAGAGTGAGATGGCCCGTATTCAGCAAGAAAATGAGAGCGCGAAG | |
| | GAAGAGGTAAAGGAAGTCTTACAAGCACTGGAAGAGCTTGCAGTGAATTAC | |
| | GATCAAAAGAGCCAGGAGATCGACAATAAGAATAAAGACATCGACGCCCTG | |
| | AACGAGGAGCTGCAGCAGAAACAATCGGTTTTCAACGCGGCGTCAACCGAG | |
| | TTGCAACAACTTAAAGATATGTCT | |
| 30. | KHMPRKMYSCDFETTTKVEDCRVWAYGYMNIEDHSEYKIGNSLDEFMAWVL | Phi29 with |
| | KVQADLYFHNLKFDGAFIINWLERNGFKWSADGLPNTYNTIISRMGQWYMI | K-linker- |
| | DICLGYKGKRKIHTVIYDSLKKLPFPVKKIAKDFKLTVLKGDIDYHKERPV | Kinesin-1 |
| | GYKITPEEYAYIKNDIQIIAEALLIQFKQGLDRMTAGSDSLKGFKDIITTK | dimerization |
| | KFKKVFPTLSLGLDKEVRYAYRGGFTWLNDRFKEKEIGEGMVFDVNSLYPA | domain long |
| | QMYSRLLPYGEPIVFEGKYVWDEDYPLHIQHIRCEFELKEGYIPTIQIKRS | (aa) |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENEKVGFSRKMKPKPVQVP | |
| | GGVVLVDDTFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELTVNY | |
| | DQKSQEIDNKNKDIDALNEELQQKQSVFNAASTELQQLKMLVPRGPVHRLG | |
| | DPPVAT | |
| 31. | KHMPRKMYSCDFETTTKVEDCRVWAYGYMNIEDHSEYKIGNSLDEFMAWVL | Phi29 with |
| | KVQADLYFHNLKEDGAFIINWLERNGFKWSADGLPNTYNTIISRMGQWYMI | K-linker- |
| | DICLGYKGKRKIHTVIYDSLKKLPFPVKKIAKDFKLTVLKGDIDYHKERPV | Kinesin-1 |
| | GYKITPEEYAYIKNDIQIIAEALLIQFKQGLDRMTAGSDSLKGFKDIITTK | dimerization |
| | KFKKVFPTLSLGLDKEVRYAYRGGFTWLNDRFKEKEIGEGMVEDVNSLYPA | domain short |
| | QMYSRLLPYGEPIVFEGKYVWDEDYPLHIQHIRCEFELKEGYIPTIQIKRS | with delta |
| | RFYKGNEYLKSSGGEIADLWLSNVDLELMKEHYDLYNVEYISGLKFKATTG | tail (aa) |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELAVNY | |
| | DQKSQEIDNKNKDIDALNEELQQKQSVENAASTELQQLKDMSRTALANMSA | |
| | SVAVNEQARLATECERLYQQLDDKDEEINQQSQYAEQLKEQVM | |
| | EQEELIANARREYETLQSEMAR | |
| 32. | KHMPRKMYSCDFETTTKVEDCRVWAYGYMNIEDHSEYKIGNSLDEFMAWVL | Phi29 with |
| | KVQADLYFHNLKEDGAFIINWLERNGFKWSADGLPNTYNTIISRMGQWYMI | K-linker- |
| | DICLGYKGKRKIHTVIYDSLKKLPFPVKKIAKDFKLTVLKGDIDYHKERPV | Kinesin-1 |
| | GYKITPEEYAYIKNDIQIIAEALLIQFKQGLDRMTAGSDSLKGFKDIITTK | dimerization |
| | KFKKVFPTLSLGLDKEVRYAYRGGFTWLNDRFKEKEIGEGMVEDVNSLYPA | domain long |
| | QMYSRLLPYGEPIVFEGKYVWDEDYPLHIQHIRCEFELKEGYIPTIQIKRS | with delta |
| | RFYKGNEYLKSSGGEIADLWLSNVDLELMKEHYDLYNVEYISGLKFKATTG | tail (aa) |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTEKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELTVNY | |
| | DQKSQEIDNKNKDIDALNEELQQKQSVENAASTELQQLKMLVPRGPVHRLG | |
| | DPPVATRTALANMSASVAVNEQARLATECERLYQQLDDKDEEINQQSQYAE | |
| | QLKEQVM EQEELIANARREYETLQSEMAR | |
| 33. | SKHMPRKMYSCDFETTTKVEDCRVWAYGYMNIEDHSEYKIGNSLDEFMAWV | Phi29 with |
| | LKVQADLYFHNLKEDGAFIINWLERNGFKWSADGLPNTYNTIISRMGQWYM | SK-linker- |
| | IDICLGYKGKRKIHTVIYDSLKKLPFPVKKIAKDFKLTVLKGDIDYHKERP | Kinesin-1 |
| | VGYKITPEEYAYIKNDIQIIAEALLIQFKQGLDRMTAGSDSLKGFKDIITT | dimerization |
| | KKFKKVFPTLSLGLDKEVRYAYRGGFTWLNDRFKEKEIGEGMVEDVNSLYP | domain long |
| | AQMYSRLLPYGEPIVFEGKYVWDEDYPLHIQHIRCEFELKEGYIPTIQIKR | (aa) |
| | SRFYKGNEYLKSSGGEIADLWLSNVDLELMKEHYDLYNVEYISGLKFKATT | |
| | GLFKDFIDKWTYIKTTSEGAIKQLAKLMLNSLYGKFASNPDVTGKVPYLKE | |
| | NGALGFRLGEEETKDPVYTPMGVFITAWARYTTITAAQACYDRIIYCDTDS | |
| | IHLTGTEIPDVIKDIVDPKKLGYWAHESTFKRAKYLRQKTYIQDIYMKEVD | |
| | GKLVEGSPDDYTDIKFSVKCAGMTDKIKKEVTFENEKVGFSRKMKPKPVQV | |
| | PGGVVLVDDTFTIKGGSKTVKNVVCVNEELTAEEWKRRYEKEKEKNARLKG | |
| | KVEKLEIELARWRAGETVKAEEQINMEDLMEASTPNLEVEAAQTAAAEAAL | |
| | AAQRTALANMSASVAVNEQARLATECERLYQQLDDKDEEINQQSQYAEQLK | |
| | EQVMEQEELIANARREYETLQSEMARIQQENESAKEEVKEVLQALEELTVN | |
| | YDQKSQEIDNKNKDIDALNEELQQKQSVENAASTELQQLKMLVPRGPVHRL | |
| | GDPPVAT | |
| 34. | KHMPRKMYSCDFETTTKVEDCRVWAYGYMNIEDHSEYKIGNSLDEFMAWVL | Phi29 with |
| | KVQADLYFHNLKEDGAFIINWLERNGFKWSADGLPNTYNTIISRMGQWYMI | K-linker- |
| | DICLGYKGKRKIHTVIYDSLKKLPFPVKKIAKDFKLTVLKGDIDYHKERPV | SpyCatcher00 |
| | GYKITPEEYAYIKNDIQIIAEALLIQFKQGLDRMTAGSDSLKGFKDIITTK | 3 (aa) |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSGGSVTTLSGLSGEQGPSGDMTTEEDSATHIKESKR | |
| | DEDGRELAGATMELRDSSGKTISTWISDGHVKDFYLYPGKYTFVETAAPDG | |
| | YEVATPIEFTVNEDGQVTVDGEATEGDAHT | |
| 35. | AAGCACATGCCACGTAAAATGTATAGCTGTGATTTTGAGACTACGACGAAA | Phi29 with |
| | GTTGAAGATTGCCGTGTGTGGGCGTATGGTTACATGAATATTGAAGATCAC | K-linker- |
| | TCCGAGTATAAGATTGGCAATAGCCTGGATGAATTCATGGCGTGGGTGCTG | SpyCatcher00 |
| | AAGGTTCAGGCCGATTTGTACTTTCATAATCTGAAATTTGATGGCGCTTTT | 3 (nt) |
| | ATCATTAACTGGCTGGAGCGTAATGGTTTCAAGTGGAGCGCAGATGGCCTG | |
| | CCGAATACGTACAACACCATTATCAGCCGTATGGGCCAGTGGTACATGATC | |
| | GACATCTGCCTGGGCTATAAGGGTAAGCGTAAGATCCACACCGTGATTTAT | |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAGGGTGGCAGCGGT | |
| | GGCAGCGTGACTACTTTATCCGGTTTGTCTGGAGAGCAAGGGCCATCCGGG | |
| | GATATGACCACAGAAGAGGATTCAGCCACACACATCAAGTTCTCAAAACGC | |
| | GACGAAGATGGCCGTGAATTGGCGGGTGCGACTATGGAACTTCGTGACTCA | |
| | TCCGGCAAGACGATTAGCACATGGATTAGTGATGGTCATGTGAAAGACTTT | |
| | TATCTGTACCCAGGGAAGTACACTTTTGTCGAGACCGCTGCTCCCGATGGT | |
| | TACGAAGTCGCTACGCCTATTGAATTTACTGTTAATGAAGATGGTCAGGTG | |
| | ACGGTCGATGGAGAAGCGACAGAGGGCGATGCGCACACT | |
| 36. | KHMPRKMYSCDFETTTKVEDCRVWAYGYMNIEDHSEYKIGNSLDEFMAWVL | Phi29 with |
| | KVQADLYFHNLKEDGAFIINWLERNGFKWSADGLPNTYNTIISRMGQWYMI | K-linker- |
| | DICLGYKGKRKIHTVIYDSLKKLPFPVKKIAKDEKLTVLKGDIDYHKERPV | SpyTag003 |
| | GYKITPEEYAYIKNDIQIIAEALLIQFKQGLDRMTAGSDSLKGFKDIITTK | (aa) |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSGGSRGVPHIVMVDAYKRYK | |
| 37. | AAGCACATGCCACGTAAAATGTATAGCTGTGATTTTGAGACTACGACGAAA | Phi29 with |
| | GTTGAAGATTGCCGTGTGTGGGCGTATGGTTACATGAATATTGAAGATCAC | K-linker- |
| | TCCGAGTATAAGATTGGCAATAGCCTGGATGAATTCATGGCGTGGGTGCTG | SpyTag003 |
| | AAGGTTCAGGCCGATTTGTACTTTCATAATCTGAAATTTGATGGCGCTTTT | (nt) |
| | ATCATTAACTGGCTGGAGCGTAATGGTTTCAAGTGGAGCGCAGATGGCCTG | |
| | CCGAATACGTACAACACCATTATCAGCCGTATGGGCCAGTGGTACATGATC | |
| | GACATCTGCCTGGGCTATAAGGGTAAGCGTAAGATCCACACCGTGATTTAT | |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAGGGTGGCAGCGGT | |
| | GGCAGCCGCGGAGTTCCACACATCGTCATGGTGGATGCGTATAAGCGTTAC | |
| | AAG | |
| 38. | KHMPRKMYSCDFETTTKVEDCRVWAYGYMNIEDHSEYKIGNSLDEFMAWVL | Phi29 with |
| | KVQADLYFHNLKEDGAFIINWLERNGFKWSADGLPNTYNTIISRMGQWYMI | K-linker-HU |
| | DICLGYKGKRKIHTVIYDSLKKLPFPVKKIAKDFKLTVLKGDIDYHKERPV | (aa) |
| | GYKITPEEYAYIKNDIQIIAEALLIQFKQGLDRMTAGSDSLKGFKDIITTK | |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENEKVGFSRKMKPKPVQVP | |
| | GGVVLVDDTFTIKGGSGGSMNKTQLIDVIAEKAELSKTQAKAALESTLAAI | |
| | TESLKEGDAVQLVGFGTFKVNHRAERTGRNPQTGKEIKIAAANVPAFVSGK | |
| | ALKDAVK | |
| 39. | AAGCACATGCCACGTAAAATGTATAGCTGTGATTTTGAGACTACGACGAAA | Phi29 with |
| | GTTGAAGATTGCCGTGTGTGGGCGTATGGTTACATGAATATTGAAGATCAC | K-linker-HU |
| | TCCGAGTATAAGATTGGCAATAGCCTGGATGAATTCATGGCGTGGGTGCTG | (nt) |
| | AAGGTTCAGGCCGATTTGTACTTTCATAATCTGAAATTTGATGGCGCTTTT | |
| | ATCATTAACTGGCTGGAGCGTAATGGTTTCAAGTGGAGCGCAGATGGCCTG | |
| | CCGAATACGTACAACACCATTATCAGCCGTATGGGCCAGTGGTACATGATC | |
| | GACATCTGCCTGGGCTATAAGGGTAAGCGTAAGATCCACACCGTGATTTAT | |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAGGGTGGCAGCGGT | |
| | GGCAGCATGAATAAGACGCAACTGATTGACGTCATTGCAGAGAAAGCCGAG | |
| | TTGAGCAAGACCCAGGCGAAAGCCGCGCTGGAGAGCACGCTGGCAGCGATT | |
| | ACCGAGAGCCTGAAAGAGGGCGATGCAGTTCAACTGGTTGGCTTCGGCACC | |
| | TTCAAGGTCAACCACCGTGCGGAGCGCACCGGTCGCAACCCGCAGACCGGT | |
| | AAAGAAATCAAGATTGCAGCCGCGAACGTGCCGGCGTTCGTCAGCGGTAAG | |
| | GCACTGAAAGATGCAGTAAAG | |
| 40. | SKHMPRKMYSCDFETTTKVEDCRVWAYGYMNIEDHSEYKIGNSLDEFMAWV | Phi29 with |
| | LKVQADLYFHNLKEDGAFIINWLERNGFKWSADGLPNTYNTIISRMGQWYM | SK-linker-HU |
| | IDICLGYKGKRKIHTVIYDSLKKLPFPVKKIAKDEKLTVLKGDIDYHKERP | (aa) |
| | VGYKITPEEYAYIKNDIQIIAEALLIQFKQGLDRMTAGSDSLKGFKDIITT | |
| | KKFKKVFPTLSLGLDKEVRYAYRGGFTWLNDRFKEKEIGEGMVEDVNSLYP | |
| | AQMYSRLLPYGEPIVFEGKYVWDEDYPLHIQHIRCEFELKEGYIPTIQIKR | |
| | SRFYKGNEYLKSSGGEIADLWLSNVDLELMKEHYDLYNVEYISGLKFKATT | |
| | GLFKDFIDKWTYIKTTSEGAIKQLAKLMLNSLYGKFASNPDVTGKVPYLKE | |
| | NGALGFRLGEEETKDPVYTPMGVFITAWARYTTITAAQACYDRIIYCDTDS | |
| | IHLTGTEIPDVIKDIVDPKKLGYWAHESTFKRAKYLRQKTYIQDIYMKEVD | |
| | GKLVEGSPDDYTDIKFSVKCAGMTDKIKKEVTFENEKVGFSRKMKPKPVQV | |
| | PGGVVLVDDTFTIKGGSMNKTQLIDVIAEKAELSKTQAKAALESTLAAITE | |
| | SLKEGDAVQLVGFGTFKVNHRAERTGRNPQTGKEIKIAAANVPAFVSGKAL | |
| | KDAVK | |
| 41. | SDSEVNQEAKPEVKPEVKPETHINLKVSDGSSEIFFKIKKTTPLRRLMEAF | SUMO tag |
| | AKRQGKEMDSLRFLYDGIRIQADQTPEDLDMEDNDIIEAHREQIGG | (aa) |
| 42. | AGCGACAGCGAAGTCAATCAAGAGGCGAAGCCTGAAGTTAAGCCAGAAGTC | SUMO tag |
| | AAACCGGAAACGCACATTAACCTGAAAGTTTCGGATGGCTCAAGCGAGATT | (nt) |
| | TTCTTTAAGATCAAGAAAACCACGCCGTTGCGTCGCCTGATGGAAGCGTTT | |
| | GCCAAACGTCAGGGCAAAGAAATGGATTCCCTGCGCTTCCTGTATGACGGC | |
| | ATCCGTATCCAGGCAGATCAAACGCCGGAAGATCTGGATATGGAAGATAAC | |
| | GACATTATTGAAGCGCACAGAGAGCAAATTGGTGGC | |
| 43. | HHHHHH | 6X HIS tag |
| 44. | MGSSHHHHHHSSG | His tag (aa) |
| 45. | ATGGGTAGCTCACATCATCATCACCATCATAGCAGCGGT | His tag (nt) |
| 46. | S(E/D) NKM (E/D) PG, WHEREIN S IS SERINE, (E/D) IS | SETtag |
| | ASPARTATE OR GLUTAMATE, K IS LYSINE, G IS GLYCINE, | consensus |
| | N IS 5 OR 6, M IS 1 OR 0, AND P IS AN INTEGER | |
| | BETWEEN 2 AND 6. | |
| 47. | SEEDEEKEEDG | SETtag |
| 48. | SSG | linker (aa) |
| 49. | KHMPRKMYSCDFETTTKVEDCRVWAYGYMNIEDHSEYKIGNSLDEFMAWVL | Phi29 A68S |
| | KVQADLYFHNLKEDGSFIINWLERNGFKWSEDGLPNTYNTIISRMGQWYMI | A83E with K- |
| | DICLGYKGKRKIHTVIYDSLKKLPFPVKKIAKDFKLTVLKGDIDYHKERPV | linker- |
| | GYKITPEEYAYIKNDIQIIAEALLIQFKQGLDRMTAGSDSLKGFKDIITTK | Kinesin-1 |
| | KFKKVFPTLSLGLDKEVRYAYRGGFTWLNDRFKEKEIGEGMVEDVNSLYPA | dimerization |
| | QMYSRLLPYGEPIVFEGKYVWDEDYPLHIQHIRCEFELKEGYIPTIQIKRS | domain short |
| | RFYKGNEYLKSSGGEIADLWLSNVDLELMKEHYDLYNVEYISGLKFKATTG | (aa) |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDDTFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELAVNY | |
| | DQKSQEIDNKNKDIDALNEELQQKQSVENAASTELQQLKDMS | |
| 50. | AAGCACATGCCACGTAAAATGTATAGCTGTGATTTTGAGACTACGACGAAA | Phi29 A68S |
| | GTTGAAGATTGCCGTGTGTGGGCGTATGGTTACATGAATATTGAAGATCAC | A83E with K- |
| | TCCGAGTATAAGATTGGCAATAGCCTGGATGAATTCATGGCGTGGGTGCTG | linker- |
| | AAGGTTCAGGCCGATTTGTACTTTCATAATCTGAAATTTGATGGCAGCTTT | Kinesin-1 |
| | ATCATTAACTGGCTGGAGCGTAATGGTTTCAAGTGGAGCGAAGATGGCCTG | dimerization |
| | CCGAATACGTACAACACCATTATCAGCCGTATGGGCCAGTGGTACATGATC | domain |
| | GACATCTGCCTGGGCTATAAGGGTAAGCGTAAGATCCACACCGTGATTTAT | short (nt) |
| | GACTCCCTGAAAAAGCTGCCGTTTCCGGTGAAGAAAATTGCCAAAGACTTC | |
| | AAGCTGACGGTTCTGAAGGGCGACATCGATTATCACAAGGAACGTCCGGTT | |
| | GGTTACAAGATCACCCCGGAAGAATACGCGTACATCAAAAACGACATTCAA | |
| | ATCATCGCAGAGGCCTTGCTGATTCAGTTCAAGCAAGGTCTGGACCGCATG | |
| | ACTGCCGGTAGCGATTCCCTGAAGGGTTTCAAAGACATTATCACCACCAAA | |
| | AAGTTCAAAAAAGTGTTCCCGACGCTGAGCCTGGGTCTGGATAAAGAGGTC | |
| | CGTTACGCTTATCGTGGCGGCTTCACCTGGTTGAACGATCGTTTCAAAGAA | |
| | AAAGAAATTGGTGAGGGCATGGTTTTTGATGTTAACTCACTGTACCCGGCA | |
| | CAAATGTATAGCCGCCTGCTGCCGTATGGCGAGCCGATCGTGTTCGAGGGT | |
| | AAATACGTGTGGGACGAAGATTACCCGCTGCATATTCAACATATCCGCTGC | |
| | GAGTTCGAGCTGAAGGAAGGCTACATCCCGACCATTCAGATTAAGCGTAGC | |
| | CGTTTTTACAAAGGTAATGAATACTTGAAGTCCTCGGGCGGTGAGATTGCG | |
| | GATCTGTGGTTAAGCAACGTCGACCTTGAGCTGATGAAAGAGCACTATGAC | |
| | CTCTATAACGTTGAGTACATTAGCGGTCTGAAATTCAAAGCCACGACGGGT | |
| | CTGTTTAAAGACTTCATTGACAAGTGGACCTATATCAAGACGACGAGCGAG | |
| | GGCGCGATCAAACAGCTGGCGAAGCTGATGCTGAATTCTCTGTACGGTAAG | |
| | TTTGCTAGCAATCCAGATGTCACCGGCAAAGTGCCGTATCTGAAAGAAAAC | |
| | GGTGCGTTGGGTTTTCGCCTGGGTGAAGAGGAAACCAAAGATCCGGTGTAC | |
| | ACCCCGATGGGCGTTTTCATTACCGCGTGGGCTCGTTACACCACGATTACC | |
| | GCAGCGCAGGCATGTTATGACCGTATCATTTACTGTGATACGGATAGCATT | |
| | CACTTGACCGGTACCGAGATCCCAGATGTCATCAAAGATATTGTCGACCCG | |
| | AAGAAACTGGGTTACTGGGCCCACGAAAGCACTTTCAAACGCGCAAAGTAT | |
| | CTGCGTCAGAAAACGTACATCCAAGACATTTACATGAAAGAGGTTGACGGC | |
| | AAATTGGTCGAGGGTTCGCCGGACGACTACACCGACATCAAGTTCAGCGTG | |
| | AAGTGCGCGGGTATGACCGACAAAATCAAAAAAGAAGTCACCTTTGAGAAC | |
| | TTTAAGGTTGGTTTTAGCCGCAAGATGAAGCCTAAACCGGTTCAGGTCCCG | |
| | GGTGGCGTTGTGCTGGTCGACGACACCTTTACTATCAAGGGTGGTTCCAAG | |
| | ACAGTAAAGAACGTTGTATGTGTCAATGAGGAACTGACCGCCGAAGAGTGG | |
| | AAACGCCGCTACGAGAAAGAGAAAGAGAAGAATGCGCGTTTAAAGGGTAAG | |
| | GTGGAGAAACTGGAAATCGAATTAGCTCGTTGGCGCGCTGGCGAGACCGTG | |
| | AAAGCCGAAGAGCAAATCAATATGGAAGATCTTATGGAAGCCTCAACTCCT | |
| | AATCTGGAAGTGGAAGCGGCTCAAACCGCAGCAGCAGAGGCTGCACTGGCG | |
| | GCCCAGCGCACCGCACTCGCAAACATGTCTGCGTCCGTGGCGGTAAATGAA | |
| | CAGGCACGTCTGGCAACCGAATGTGAACGCCTTTATCAACAATTGGACGAC | |
| | AAAGACGAGGAGATCAACCAACAAAGTCAGTACGCAGAGCAATTGAAAGAA | |
| | CAAGTTATGGAGCAAGAAGAGTTGATTGCGAATGCGCGCCGTGAGTACGAG | |
| | ACTCTTCAGAGTGAGATGGCCCGTATTCAGCAAGAAAATGAGAGCGCGAAG | |
| | GAAGAGGTAAAGGAAGTCTTACAAGCACTGGAAGAGCTTGCAGTGAATTAC | |
| | GATCAAAAGAGCCAGGAGATCGACAATAAGAATAAAGACATCGACGCCCTG | |
| | AACGAGGAGCTGCAGCAGAAACAATCGGTTTTCAACGCGGCGTCAACCGAG | |
| | TTGCAACAACTTAAAGATATGTCT | |
| 51. | KHMPRKMYSCDFETTTKVEDCRVWAYGYMNIEDHSEYKIGNSLDEFMAWVL | Phi29 D570S |
| | KVQADLYFHNLKFDGAFIINWLERNGFKWSADGLPNTYNTIISRMGQWYMI | T571V (aa) |
| | DICLGYKGKRKIHTVIYDSLKKLPFPVKKIAKDEKLTVLKGDIDYHKERPV | |
| | GYKITPEEYAYIKNDIQIIAEALLIQFKQGLDRMTAGSDSLKGFKDIITTK | |
| | KFKKVFPTLSLGLDKEVRYAYRGGFTWLNDRFKEKEIGEGMVEDVNSLYPA | |
| | QMYSRLLPYGEPIVFEGKYVWDEDYPLHIQHIRCEFELKEGYIPTIQIKRS | |
| | RFYKGNEYLKSSGGEIADLWLSNVDLELMKEHYDLYNVEYISGLKFKATTG | |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENFKVGFSRKMKPKPVQVP | |
| | GGVVLVDSVFTIK | |
| 52. | KHMPRKMYSCDFETTTKVEDCRVWAYGYMNIEDHSEYKIGNSLDEFMAWVL | Phi29 D570S |
| | KVQADLYFHNLKEDGAFIINWLERNGFKWSADGLPNTYNTIISRMGQWYMI | T571V with |
| | DICLGYKGKRKIHTVIYDSLKKLPFPVKKIAKDFKLTVLKGDIDYHKERPV | K-linker- |
| | GYKITPEEYAYIKNDIQIIAEALLIQFKQGLDRMTAGSDSLKGFKDIITTK | Kinesin-1 |
| | KFKKVFPTLSLGLDKEVRYAYRGGFTWLNDRFKEKEIGEGMVEDVNSLYPA | dimerization |
| | QMYSRLLPYGEPIVFEGKYVWDEDYPLHIQHIRCEFELKEGYIPTIQIKRS | domain short |
| | RFYKGNEYLKSSGGEIADLWLSNVDLELMKEHYDLYNVEYISGLKFKATTG | (aa) |
| | LFKDFIDKWTYIKTTSEGAIKQLAKLMLNSLYGKFASNPDVTGKVPYLKEN | |
| | GALGFRLGEEETKDPVYTPMGVFITAWARYTTITAAQACYDRIIYCDTDSI | |
| | HLTGTEIPDVIKDIVDPKKLGYWAHESTFKRAKYLRQKTYIQDIYMKEVDG | |
| | KLVEGSPDDYTDIKFSVKCAGMTDKIKKEVTFENEKVGFSRKMKPKPVQVP | |
| | GGVVLVDSVFTIKGGSKTVKNVVCVNEELTAEEWKRRYEKEKEKNARLKGK | |
| | VEKLEIELARWRAGETVKAEEQINMEDLMEASTPNLEVEAAQTAAAEAALA | |
| | AQRTALANMSASVAVNEQARLATECERLYQQLDDKDEEINQQSQYAEQLKE | |
| | QVMEQEELIANARREYETLQSEMARIQQENESAKEEVKEVLQALEELAVNY | |
| | DQKSQEIDNKNKDIDALNEELQQKQSVENAASTELQQLKDMS | |

1-144. (canceled)

145. A fusion protein comprising a Phi29 polymerase domain and one or more protein-protein interaction domains.

146. The fusion protein of claim 145, wherein the one or more protein-protein interaction domains comprise one or more dimerization domains.

147. The fusion protein of claim 146, wherein the dimerization domain comprises a kinesin 1 dimerization domain.

148. The fusion protein of claim 147, wherein the kinesin 1 dimerization comprises the sequence set forth in SEQ ID NO:18 or 20 or a sequence that has at least at or about 95% sequence identity to the sequence set forth in SEQ ID NO: 18 or 20.

149. The fusion protein of claim 146, wherein when a first fusion protein molecule and a second fusion protein molecule are present in proximity, the dimerization domains interact to produce a dimer comprising the first fusion protein molecule and the second fusion protein molecule.

150. The fusion protein of claim 145, wherein the one or more protein-protein interaction domains comprises a SpyCatcher domain or a SpyTag domain.

151. The fusion protein of claim 145, wherein the one or more protein-protein interaction domains comprises a SpyCatcher domain, and wherein the SpyCatcher domain comprises the sequence set forth in SEQ ID NO:22 or a sequence that has at least at or about 95% sequence identity to the sequence set forth in SEQ ID NO:22.

152. The fusion protein of claim 145, wherein the one or more protein-protein interaction domains comprises a SpyTag domain, and wherein the SpyTag domain comprises the sequence set forth in SEQ ID NO:24 or a sequence that has at least at or about 95% sequence identity to the sequence set forth in SEQ ID NO:24.

153. The fusion protein of claim 150, wherein when a first fusion protein molecule comprising a SpyCatcher domain and a second fusion protein molecule comprising a SpyTag domain are present in proximity, the SpyCatcher domain and the SpyTag domain interact to form a covalent bond to produce a dimer comprising the first fusion protein molecule and the second fusion protein molecule.

154. The fusion protein of claim 145, wherein the Phi29 polymerase domain comprises the sequence set forth in SEQ ID NO:3 or a sequence that has at least at or about 95%, sequence identity to the sequence set forth in SEQ ID NO:3.

155. The fusion protein of claim 145, wherein the Phi29 polymerase domain comprises one or more amino acid substitutions at one or more positions selected from among positions 68 and 83, corresponding to the positions of the sequence set forth in SEQ ID NO:1.

156. The fusion protein of claim 145, wherein the fusion protein further comprises one or more heterologous nucleic acid binding domains, linkers, and/or tags.

157. A fusion protein comprising a Phi29 polymerase domain and one or more heterologous nucleic acid binding domains.

158. The fusion protein of claim 157, wherein the one or more heterologous nucleic acid binding domains comprises a HU domain.

159. The fusion protein claim 145, wherein the fusion protein further comprises a tag.

160. The fusion protein of claim 159, wherein the tag is selected from among a poly histidine (HIS) tag, a solubility enhancement tag (SET), a small ubiquitin modified (SUMO) tag, a Fasciola hepatica 8-kDa antigen (Fh8) tag, a thioredoxin (Trx) tag, a glutathione-s-transferase (GST) tag, a maltose-binding protein (MBP) tag, an IgG domain B1 of protein G (GB1) tag, a mutated dehalogenase (Halo) tag, a steptavidin-binding peptide (SBP) tag, and a Tamavidin tag.

161. The fusion protein of claim 145, wherein the fusion protein comprises the sequence set forth in SEQ ID NO:28 or 33 or has at least at or about 95% sequence identity to the sequence set forth in SEQ ID NO:28 or 33.

162. The fusion protein of claim 145, wherein the fusion protein comprises the sequence set forth in SEQ ID NO:34 or 36 or has at least at or about 95% sequence identity to the sequence set forth in SEQ ID NO:34 or 36.

163. The fusion protein of claim 157, wherein the fusion protein comprises the sequence set forth in SEQ ID NO:38 or has at least at or about 95% sequence identity to the sequence set forth in SEQ ID NO:38.

164. The fusion protein of claim 145, wherein the fusion protein:
(i) generates a higher signal intensity of RCA products in an RCA compared to a reference Phi29 polymerase;
(ii) generates a smaller or more compact RCA product compared to a referenced Phi29 polymerase;
(iii) generates a higher density of detected rolling-circle amplification (RCA) products in an RCA compared to a reference Phi29 polymerase;
(iv) exhibits improved nucleic acid binding affinity compared to a reference Phi29 polymerase; and/or
(v) exhibits improved processivity compared to a reference Phi29 polymerase wherein, the reference Phi29 polymerase is a wild-type Phi29 polymerase, or a Phi29 polymerase that does not comprise the one or more protein-protein interaction domains.


SEQUENCE LISTING (52 sequences)

SEQ ID NO: 1 - protein, 575 aa, synthetic construct [sequence omitted]

SEQ ID NO: 2 - DNA, 1725 bp, synthetic construct
  source 1..1725: mol_type = other DNA
atgaagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcgcttttatcattaactggctggagcgtaatggtttcaagtggagcgcagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaag

SEQ ID NO: 3 - protein, 574 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 4 - DNA, 1722 bp, synthetic construct
  source 1..1722: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcgcttttatcattaactggctggagcgtaatggtttcaagtggagcgcagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaag

SEQ ID NO: 5 - protein, 576 aa, synthetic construct
MSKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 6 - protein, 575 aa, synthetic construct
SKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 7 - protein, 574 aa, synthetic construct
KHMPRKMYSCDFETTTKVEDCRVWAYGYMNIEDHSEYKIGNSLDEFMAWVLKVQADLYFHNLKFDGSFIINWLERNGFKWSEDGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 8 - DNA, 1722 bp, synthetic construct
  source 1..1722: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcagctttatcattaactggctggagcgtaatggtttcaagtggagcgaagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaag

SEQ ID NO: 9 - skipped

SEQ ID NO: 10 - protein, 6 aa, synthetic construct
GGSGGS

SEQ ID NO: 11 - DNA, 18 bp, synthetic construct
  source 1..18: mol_type = other DNA
ggtggcagcggtggcagc

SEQ ID NO: 12 - protein, 5 aa, synthetic construct
GGSGG

SEQ ID NO: 13 - protein, 5 aa, synthetic construct
GGGGG

SEQ ID NO: 14 - protein, 5 aa, synthetic construct
GGAGG

SEQ ID NO: 15 - protein, 7 aa, synthetic construct
GGGGSSS

SEQ ID NO: 16 - protein, 7 aa, synthetic construct
GGGGAAA

SEQ ID NO: 17 - protein, 5 aa, synthetic construct
  VARIANT 1..5: note = Can be present in repeats of up to 10
GGGGS

SEQ ID NO: 18 - protein, 230 aa, synthetic construct
KTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMS

SEQ ID NO: 19 - DNA, 690 bp, synthetic construct
  source 1..690: mol_type = other DNA
aagacagtaaagaacgttgtatgtgtcaatgaggaactgaccgccgaagagtggaaacgccgctacgagaaagagaaagagaagaatgcgcgtttaaagggtaaggtggagaaactggaaatcgaattagctcgttggcgcgctggcgagaccgtgaaagccgaagagcaaatcaatatggaagatcttatggaagcctcaactcctaatctggaagtggaagcggctcaaaccgcagcagcagaggctgcactggcggcccagcgcaccgcactcgcaaacatgtctgcgtccgtggcggtaaatgaacaggcacgtctggcaaccgaatgtgaacgcctttatcaacaattggacgacaaagacgaggagatcaaccaacaaagtcagtacgcagagcaattgaaagaacaagttatggagcaagaagagttgattgcgaatgcgcgccgtgagtacgagactcttcagagtgagatggcccgtattcagcaagaaaatgagagcgcgaaggaagaggtaaaggaagtcttacaagcactggaagagcttgcagtgaattacgatcaaaagagccaggagatcgacaataagaataaagacatcgacgccctgaacgaggagctgcagcagaaacaatcggttttcaacgcggcgtcaaccgagttgcaacaacttaaagatatgtct

SEQ ID NO: 20 - protein, 245 aa, synthetic construct
KTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELTVNYDQKSQEIDNKNKDIDALNEELQQKQSVFNAASTELQQLKMLVPRGPVHRLGDPPVAT

SEQ ID NO: 21 - protein, 74 aa, synthetic construct
RTALANMSASVAVNEQARLATECERLYQQLDDKDEEINQQSQYAEQLKEQVMEQEELIANARREYETLQSEMAR

SEQ ID NO: 22 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 23 - DNA, 339 bp, synthetic construct
  source 1..339: mol_type = other DNA
gtgactactttatccggtttgtctggagagcaagggccatccggggatatgaccacagaagaggattcagccacacacatcaagttctcaaaacgcgacgaagatggccgtgaattggcgggtgcgactatggaacttcgtgactcatccggcaagacgattagcacatggattagtgatggtcatgtgaaagacttttatctgtacccagggaagtacacttttgtcgagaccgctgctcccgatggttacgaagtcgctacgcctattgaatttactgttaatgaagatggtcaggtgacggtcgatggagaagcgacagagggcgatgcgcacact

SEQ ID NO: 24 - protein, 16 aa, synthetic construct
RGVPHIVMVDAYKRYK

SEQ ID NO: 25 - DNA, 48 bp, synthetic construct
  source 1..48: mol_type = other DNA
cgcggagttccacacatcgtcatggtggatgcgtataagcgttacaag

SEQ ID NO: 26 - protein, 90 aa, synthetic construct
MNKTQLIDVIAEKAELSKTQAKAALESTLAAITESLKEGDAVQLVGFGTFKVNHRAERTGRNPQTGKEIKIAAANVPAFVSGKALKDAVK

SEQ ID NO: 27 - DNA, 270 bp, synthetic construct
  source 1..270: mol_type = other DNA
atgaataagacgcaactgattgacgtcattgcagagaaagccgagttgagcaagacccaggcgaaagccgcgctggagagcacgctggcagcgattaccgagagcctgaaagagggcgatgcagttcaactggttggcttcggcaccttcaaggtcaaccaccgtgcggagcgcaccggtcgcaacccgcagaccggtaaagaaatcaagattgcagccgcgaacgtgccggcgttcgtcagcggtaaggcactgaaagatgcagtaaag

SEQ ID NO: 28 - protein, 807 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMS

SEQ ID NO: 29 - DNA, 2421 bp, synthetic construct
  source 1..2421: mol_type = other DNA
aagcacatgcctcgcaagatgtacagctgcgattttgagaccactacaaaggttgaagactgccgtgtctgggcgtatgggtatatgaatattgaagaccacagcgagtacaagattggtaacagcttggacgaattcatggcttgggttctcaaggttcaagcagatctttatttccataacctcaagtttgatggcgcgtttatcattaactggttggagcgtaatggatttaagtggagtgccgacggtttgccgaacacctataacacgatcatctctcgcatgggtcaatggtatatgatcgacatttgtctgggctacaagggcaagcgtaagattcatacagtcatctacgactcacttaagaaattgcctttccctgttaagaagatcgcgaaagacttcaagttgaccgttcttaagggcgacattgactatcacaaagagcgtccggtgggatataaaatcactccagaagagtacgcttacatcaagaacgacatccagattatcgcagaggcgttactcatccagttcaaacaagggctggatcgtatgaccgctggctcagacagtttaaagggcttcaaagacattatcactacgaagaaattcaagaaggtctttcctaccttgagtctcggactggacaaagaggtgcgttacgcctatcgcggcggtttcacttggcttaacgaccgtttcaaagagaaagagattggcgaaggtatggtgttcgacgtgaatagtctctacccggcgcagatgtattcccgcttgttaccgtacggtgaaccgattgtgttcgaggggaagtatgtctgggacgaagactaccctttacacatccagcatattcgttgcgagtttgagttaaaagagggttacatcccgaccattcaaattaagcgtagtcgtttctataaagggaatgagtacttgaagtctagcggcggcgagattgctgacctttggctttcaaacgtcgacctggagcttatgaaagagcactatgatttgtacaacgtggaatatatcagcgggctcaagttcaaagcgaccacagggttattcaaggatttcattgataagtggacttatatcaaaacgacgagtgagggcgcaatcaaacaattggcgaagctcatgcttaacagcttgtacggaaagtttgcttcgaacccagatgtaacaggcaaggtcccgtacctcaaagaaaatggcgcattaggcttccgcctgggtgaagaggaaacgaaagaccctgtttatacccctatgggcgtatttattaccgcctgggcgcgctacacaactattaccgcggcgcaggcctgctatgaccgtattatttactgcgacaccgacagcatccacctcaccgggacggagatcccagacgtcatcaaggatattgtggacccgaagaagttgggctattgggcgcatgagtcaacctttaaacgtgccaaatatctgcgtcaaaagacctacatccaagatatctatatgaaggaagttgacggtaagttggttgaaggtagtccggacgattatacagacattaaatttagcgtaaagtgcgcgggcatgacagacaaaattaagaaggaagtaacgttcgaaaattttaaggttggtttctcgcgcaagatgaagcctaaaccggtgcaagtccctggtggggttgtccttgttgatgacacctttaccattaagggtggttccaagacagtaaagaacgttgtatgtgtcaatgaggaactgaccgccgaagagtggaaacgccgctacgagaaagagaaagagaagaatgcgcgtttaaagggtaaggtggagaaactggaaatcgaattagctcgttggcgcgctggcgagaccgtgaaagccgaagagcaaatcaatatggaagatcttatggaagcctcaactcctaatctggaagtggaagcggctcaaaccgcagcagcagaggctgcactggcggcccagcgcaccgcactcgcaaacatgtctgcgtccgtggcggtaaatgaacaggcacgtctggcaaccgaatgtgaacgcctttatcaacaattggacgacaaagacgaggagatcaaccaacaaagtcagtacgcagagcaattgaaagaacaagttatggagcaagaagagttgattgcgaatgcgcgccgtgagtacgagactcttcagagtgagatggcccgtattcagcaagaaaatgagagcgcgaaggaagaggtaaaggaagtcttacaagcactggaagagcttgcagtgaattacgatcaaaagagccaggagatcgacaataagaataaagacatcgacgccctgaacgaggagctgcagcagaaacaatcggttttcaacgcggcgtcaaccgagttgcaacaacttaaagatatgtct

SEQ ID NO: 30 - protein, 822 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELTVNYDQKSQEIDNKNKDIDALNEELQQKQSVFNAASTELQQLKMLVPRGPVHRLGDPPVAT

SEQ ID NO: 31 - protein, 881 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMSRTALANMSASVAVNEQARLATECERLYQQLDDKDEEINQQSQYAEQLKEQVMEQEELIANARREYETLQSEMAR

SEQ ID NO: 32 - protein, 896 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELTVNYDQKSQEIDNKNKDIDALNEELQQKQSVFNAASTELQQLKMLVPRGPVHRLGDPPVATRTALANMSASVAVNEQARLATECERLYQQLDDKDEEINQQSQYAEQLKEQVMEQEELIANARREYETLQSEMAR

SEQ ID NO: 33 - protein, 823 aa, synthetic construct
SKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELTVNYDQKSQEIDNKNKDIDALNEELQQKQSVFNAASTELQQLKMLVPRGPVHRLGDPPVAT

SEQ ID NO: 34 - protein, 693 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSGGSVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 35 - DNA, 2079 bp, synthetic construct
  source 1..2079: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcgcttttatcattaactggctggagcgtaatggtttcaagtggagcgcagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaagggtggcagcggtggcagcgtgactactttatccggtttgtctggagagcaagggccatccggggatatgaccacagaagaggattcagccacacacatcaagttctcaaaacgcgacgaagatggccgtgaattggcgggtgcgactatggaacttcgtgactcatccggcaagacgattagcacatggattagtgatggtcatgtgaaagacttttatctgtacccagggaagtacacttttgtcgagaccgctgctcccgatggttacgaagtcgctacgcctattgaatttactgttaatgaagatggtcaggtgacggtcgatggagaagcgacagagggcgatgcgcacact

SEQ ID NO: 36 - protein, 596 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSGGSRGVPHIVMVDAYKRYK

SEQ ID NO: 37 - DNA, 1788 bp, synthetic construct
  source 1..1788: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcgcttttatcattaactggctggagcgtaatggtttcaagtggagcgcagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaagggtggcagcggtggcagccgcggagttccacacatcgtcatggtggatgcgtataagcgttacaag

SEQ ID NO: 38 - protein, 670 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSGGSMNKTQLIDVIAEKAELSKTQAKAALESTLAAITESLKEGDAVQLVGFGTFKVNHRAERTGRNPQTGKEIKIAAANVPAFVSGKALKDAVK

SEQ ID NO: 39 - DNA, 2010 bp, synthetic construct
  source 1..2010: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcgcttttatcattaactggctggagcgtaatggtttcaagtggagcgcagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaagggtggcagcggtggcagcatgaataagacgcaactgattgacgtcattgcagagaaagccgagttgagcaagacccaggcgaaagccgcgctggagagcacgctggcagcgattaccgagagcctgaaagagggcgatgcagttcaactggttggcttcggcaccttcaaggtcaaccaccgtgcggagcgcaccggtcgcaacccgcagaccggtaaagaaatcaagattgcagccgcgaacgtgccggcgttcgtcagcggtaaggcactgaaagatgcagtaaag

SEQ ID NO: 40 - protein, 668 aa, synthetic construct
SKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSMNKTQLIDVIAEKAELSKTQAKAALESTLAAITESLKEGDAVQLVGFGTFKVNHRAERTGRNPQTGKEIKIAAANVPAFVSGKALKDAVK

SEQ ID NO: 41 - protein, 97 aa, synthetic construct
SDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 42 - DNA, 291 bp, synthetic construct
  source 1..291: mol_type = other DNA
agcgacagcgaagtcaatcaagaggcgaagcctgaagttaagccagaagtcaaaccggaaacgcacattaacctgaaagtttcggatggctcaagcgagattttctttaagatcaagaaaaccacgccgttgcgtcgcctgatggaagcgtttgccaaacgtcagggcaaagaaatggattccctgcgcttcctgtatgacggcatccgtatccaggcagatcaaacgccggaagatctggatatggaagataacgacattattgaagcgcacagagagcaaattggtggc

SEQ ID NO: 43 - protein, 6 aa, synthetic construct
HHHHHH

SEQ ID NO: 44 - protein, 13 aa, synthetic construct
MGSSHHHHHHSSG

SEQ ID NO: 45 - DNA, 39 bp, synthetic construct
  source 1..39: mol_type = other DNA
atgggtagctcacatcatcatcaccatcatagcagcggt

SEQ ID NO: 46 - skipped

SEQ ID NO: 47 - protein, 11 aa, synthetic construct
SEEDEEKEEDG

SEQ ID NO: 48 - skipped

SEQ ID NO: 49 - protein, 807 aa, synthetic construct
KHMPRKMYSCDFETTTKVEDCRVWAYGYMNIEDHSEYKIGNSLDEFMAWVLKVQADLYFHNLKFDGSFIINWLERNGFKWSEDGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMS

SEQ ID NO: 50 - DNA, 2421 bp, synthetic construct
  source 1..2421: mol_type = other DNA
aagcacatgccacgtaaaatgtatagctgtgattttgagactacgacgaaagttgaagattgccgtgtgtgggcgtatggttacatgaatattgaagatcactccgagtataagattggcaatagcctggatgaattcatggcgtgggtgctgaaggttcaggccgatttgtactttcataatctgaaatttgatggcagctttatcattaactggctggagcgtaatggtttcaagtggagcgaagatggcctgccgaatacgtacaacaccattatcagccgtatgggccagtggtacatgatcgacatctgcctgggctataagggtaagcgtaagatccacaccgtgatttatgactccctgaaaaagctgccgtttccggtgaagaaaattgccaaagacttcaagctgacggttctgaagggcgacatcgattatcacaaggaacgtccggttggttacaagatcaccccggaagaatacgcgtacatcaaaaacgacattcaaatcatcgcagaggccttgctgattcagttcaagcaaggtctggaccgcatgactgccggtagcgattccctgaagggtttcaaagacattatcaccaccaaaaagttcaaaaaagtgttcccgacgctgagcctgggtctggataaagaggtccgttacgcttatcgtggcggcttcacctggttgaacgatcgtttcaaagaaaaagaaattggtgagggcatggtttttgatgttaactcactgtacccggcacaaatgtatagccgcctgctgccgtatggcgagccgatcgtgttcgagggtaaatacgtgtgggacgaagattacccgctgcatattcaacatatccgctgcgagttcgagctgaaggaaggctacatcccgaccattcagattaagcgtagccgtttttacaaaggtaatgaatacttgaagtcctcgggcggtgagattgcggatctgtggttaagcaacgtcgaccttgagctgatgaaagagcactatgacctctataacgttgagtacattagcggtctgaaattcaaagccacgacgggtctgtttaaagacttcattgacaagtggacctatatcaagacgacgagcgagggcgcgatcaaacagctggcgaagctgatgctgaattctctgtacggtaagtttgctagcaatccagatgtcaccggcaaagtgccgtatctgaaagaaaacggtgcgttgggttttcgcctgggtgaagaggaaaccaaagatccggtgtacaccccgatgggcgttttcattaccgcgtgggctcgttacaccacgattaccgcagcgcaggcatgttatgaccgtatcatttactgtgatacggatagcattcacttgaccggtaccgagatcccagatgtcatcaaagatattgtcgacccgaagaaactgggttactgggcccacgaaagcactttcaaacgcgcaaagtatctgcgtcagaaaacgtacatccaagacatttacatgaaagaggttgacggcaaattggtcgagggttcgccggacgactacaccgacatcaagttcagcgtgaagtgcgcgggtatgaccgacaaaatcaaaaaagaagtcacctttgagaactttaaggttggttttagccgcaagatgaagcctaaaccggttcaggtcccgggtggcgttgtgctggtcgacgacacctttactatcaagggtggttccaagacagtaaagaacgttgtatgtgtcaatgaggaactgaccgccgaagagtggaaacgccgctacgagaaagagaaagagaagaatgcgcgtttaaagggtaaggtggagaaactggaaatcgaattagctcgttggcgcgctggcgagaccgtgaaagccgaagagcaaatcaatatggaagatcttatggaagcctcaactcctaatctggaagtggaagcggctcaaaccgcagcagcagaggctgcactggcggcccagcgcaccgcactcgcaaacatgtctgcgtccgtggcggtaaatgaacaggcacgtctggcaaccgaatgtgaacgcctttatcaacaattggacgacaaagacgaggagatcaaccaacaaagtcagtacgcagagcaattgaaagaacaagttatggagcaagaagagttgattgcgaatgcgcgccgtgagtacgagactcttcagagtgagatggcccgtattcagcaagaaaatgagagcgcgaaggaagaggtaaaggaagtcttacaagcactggaagagcttgcagtgaattacgatcaaaagagccaggagatcgacaataagaataaagacatcgacgccctgaacgaggagctgcagcagaaacaatcggttttcaacgcggcgtcaaccgagttgcaacaacttaaagatatgtct

SEQ ID NO: 51 - protein, 574 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDSVFTIK

SEQ ID NO: 52 - protein, 807 aa, synthetic construct
KHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDSVFTIKGGSKTVKNVVCVNEELTAEEWKRRYEKEKEKNARLKGKVEKLEIELARWRAGETVKAEEQINMEDLMEASTPNLEVEAAQTAAAEAALAAQRTALANMSASVAVNEQARLATECERLYQQLDDKDEEINQQSQYAEQLKEQVMEQEELIANARREYETLQSEMARIQQENESAKEEVKEVLQALEELAVNYDQKSQEIDNKNKDIDALNEELQQKQSVFNAASTELQQLKDMS